SEQUENCE LISTING (182 sequences)

SEQ ID NO: 1 - protein, 447 aa, Homo sapiens
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Homo sapiens
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 454 aa, Homo sapiens
EVQLVESGGGLVQPGGSLKLSCAASGFTFNTYAMNWVRQAPGKGLEWVGRIRSKYNNYATYYADSVKDRFTISRDDSKNTAYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 216 aa, Homo sapiens
EIVLTQSPATLSLSPGERATLSCRASQSVNSNLNWYQQKPGQAPRLLIYHSNNRPHGIPARFSGSGSGTDFTLTISSLEPEDFAVYYCQQFDYFREYNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 215 aa, Homo sapiens
QTVVTQEPSLTVSPGGTVTLTCRSSTGAVTTSNYANWFQQKPGQAPRGLIGGTNKRAPGTPARFSGSLLGGKAALTLSGAQPEDEAEYYCALWYKQRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - protein, 8 aa, Homo sapiens
GFTFSSYA

SEQ ID NO: 7 - protein, 8 aa, Homo sapiens
ISGSGGST

SEQ ID NO: 8 - protein, 9 aa, Homo sapiens
AKSYGAFDY

SEQ ID NO: 9 - protein, 116 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 10 - protein, 445 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 445 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 445 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 9 aa, Homo sapiens
SSDVFRPPL

SEQ ID NO: 14 - skipped

SEQ ID NO: 15 - protein, 11 aa, Homo sapiens
SSWDHTGDGHV

SEQ ID NO: 16 - protein, 111 aa, Homo sapiens
QSALTQPASVSGSPGQSITISCTGTSSDVFRPPLVSWYQQHPGKAPKLMIYFASLRPSGVSNRFSGSKSGNTASLTISGLQAEDEADYYCSSWDHTGDGHVFGGGTKLTVL

SEQ ID NO: 17 - protein, 217 aa, Homo sapiens
QSALTQPASVSGSPGQSITISCTGTSSDVFRPPLVSWYQQHPGKAPKLMIYFASLRPSGVSNRFSGSKSGNTASLTISGLQAEDEADYYCSSWDHTGDGHVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 12 aa, Homo sapiens
QSVLYSSNNKNY

SEQ ID NO: 19 - skipped

SEQ ID NO: 20 - protein, 9 aa, Homo sapiens
QQNLRPPET

SEQ ID NO: 21 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQNLRPPETFGQGTKVEIK

SEQ ID NO: 22 - protein, 220 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQNLRPPETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 12 aa, Homo sapiens
QSVLYSSNNKNY

SEQ ID NO: 24 - skipped

SEQ ID NO: 25 - protein, 9 aa, Homo sapiens
QQNFRPPET

SEQ ID NO: 26 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVGVYYCQQNFRPPETFGGGTKVEIK

SEQ ID NO: 27 - protein, 220 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVGVYYCQQNFRPPETFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 11 aa, Homo sapiens
GDLLTFEGKTY

SEQ ID NO: 29 - skipped

SEQ ID NO: 30 - protein, 11 aa, Homo sapiens
MQAHSTRFAFT

SEQ ID NO: 31 - protein, 8 aa, Homo sapiens
SSNIGSNT

SEQ ID NO: 32 - skipped

SEQ ID NO: 33 - protein, 12 aa, Homo sapiens
AAWDLNPAIGYV

SEQ ID NO: 34 - protein, 11 aa, Homo sapiens
DDLLRFSGKTY

SEQ ID NO: 35 - skipped

SEQ ID NO: 36 - protein, 11 aa, Homo sapiens
MQASRSKVAFT

SEQ ID NO: 37 - protein, 11 aa, Homo sapiens
GDLLSFDGKTY

SEQ ID NO: 38 - skipped

SEQ ID NO: 39 - protein, 11 aa, Homo sapiens
MQASKHRFGFT

SEQ ID NO: 40 - protein, 11 aa, Homo sapiens
GDLLEFAGKTY

SEQ ID NO: 41 - skipped

SEQ ID NO: 42 - protein, 11 aa, Homo sapiens
MQAHGSKIGFT

SEQ ID NO: 43 - protein, 6 aa, Homo sapiens
QEIHSH

SEQ ID NO: 44 - skipped

SEQ ID NO: 45 - protein, 10 aa, Homo sapiens
QQGFRFAFST

SEQ ID NO: 46 - protein, 12 aa, Homo sapiens
QSILYSSSNRNY

SEQ ID NO: 47 - skipped

SEQ ID NO: 48 - protein, 9 aa, Homo sapiens
QQNFRPPET

SEQ ID NO: 49 - protein, 7 aa, Homo sapiens
QAVFSLD

SEQ ID NO: 50 - skipped

SEQ ID NO: 51 - protein, 11 aa, Homo sapiens
QQGPAFKPDVT

SEQ ID NO: 52 - protein, 6 aa, Homo sapiens
QSVSSY

SEQ ID NO: 53 - skipped

SEQ ID NO: 54 - protein, 9 aa, Homo sapiens
HQRYRPPET

SEQ ID NO: 55 - protein, 6 aa, Homo sapiens
QSIGAY

SEQ ID NO: 56 - skipped

SEQ ID NO: 57 - protein, 9 aa, Homo sapiens
QQYKDRPLT

SEQ ID NO: 58 - protein, 11 aa, Homo sapiens
GNLLTHDGKTY

SEQ ID NO: 59 - skipped

SEQ ID NO: 60 - protein, 11 aa, Homo sapiens
MQANALKFGFT

SEQ ID NO: 61 - protein, 11 aa, Homo sapiens
SDLLRFEGKTY

SEQ ID NO: 62 - skipped

SEQ ID NO: 63 - protein, 11 aa, Homo sapiens
MQAHASALKFT

SEQ ID NO: 64 - protein, 11 aa, Homo sapiens
GNLLTHGGKTY

SEQ ID NO: 65 - skipped

SEQ ID NO: 66 - protein, 11 aa, Homo sapiens
MQAHADGFGFT

SEQ ID NO: 67 - protein, 11 aa, Homo sapiens
GDLLRFDGKTY

SEQ ID NO: 68 - skipped

SEQ ID NO: 69 - protein, 11 aa, Homo sapiens
MQASETAVRFT

SEQ ID NO: 70 - protein, 11 aa, Homo sapiens
TDLLTFEGKTY

SEQ ID NO: 71 - skipped

SEQ ID NO: 72 - protein, 11 aa, Homo sapiens
MQAHSSKFKFT

SEQ ID NO: 73 - protein, 11 aa, Homo sapiens
GDLLSTDGKTY

SEQ ID NO: 74 - skipped

SEQ ID NO: 75 - protein, 11 aa, Homo sapiens
MQASGSRFYFT

SEQ ID NO: 76 - protein, 12 aa, Homo sapiens
QTLLYKYDNKNY

SEQ ID NO: 77 - skipped

SEQ ID NO: 78 - protein, 9 aa, Homo sapiens
HQYLYPAET

SEQ ID NO: 79 - protein, 8 aa, Homo sapiens
SSNIRDSF

SEQ ID NO: 80 - skipped

SEQ ID NO: 81 - protein, 9 aa, Homo sapiens
AAWHPYYTL

SEQ ID NO: 82 - protein, 8 aa, Homo sapiens
SSNIRDSF

SEQ ID NO: 83 - skipped

SEQ ID NO: 84 - protein, 9 aa, Homo sapiens
ASWWPYGTV

SEQ ID NO: 85 - protein, 8 aa, Homo sapiens
SSNIRDSF

SEQ ID NO: 86 - skipped

SEQ ID NO: 87 - protein, 9 aa, Homo sapiens
ASWWPFGTV

SEQ ID NO: 88 - protein, 9 aa, Homo sapiens
SSDVVKNNF

SEQ ID NO: 89 - skipped

SEQ ID NO: 90 - protein, 9 aa, Homo sapiens
SSWDMPALF

SEQ ID NO: 91 - protein, 9 aa, Homo sapiens
SSDVVKNNF

SEQ ID NO: 92 - skipped

SEQ ID NO: 93 - protein, 9 aa, Homo sapiens
SSWDEPDRP

SEQ ID NO: 94 - protein, 9 aa, Homo sapiens
SSDVVKNNF

SEQ ID NO: 95 - skipped

SEQ ID NO: 96 - protein, 9 aa, Homo sapiens
SSWDLPFLM

SEQ ID NO: 97 - protein, 9 aa, Homo sapiens
SSDVVKNNF

SEQ ID NO: 98 - skipped

SEQ ID NO: 99 - protein, 9 aa, Homo sapiens
SSWKEPDRP

SEQ ID NO: 100 - protein, 9 aa, Homo sapiens
SSDVVKNNF

SEQ ID NO: 101 - skipped

SEQ ID NO: 102 - protein, 9 aa, Homo sapiens
SSWDEPDRP

SEQ ID NO: 103 - protein, 8 aa, Homo sapiens
SSNIAHKP

SEQ ID NO: 104 - skipped

SEQ ID NO: 105 - protein, 13 aa, Homo sapiens
AAWDFATWPATEV

SEQ ID NO: 106 - protein, 8 aa, Homo sapiens
SSNIAHKP

SEQ ID NO: 107 - skipped

SEQ ID NO: 108 - protein, 13 aa, Homo sapiens
AAWDFSRWPATEV

SEQ ID NO: 109 - protein, 8 aa, Homo sapiens
SVDIAHKP

SEQ ID NO: 110 - skipped

SEQ ID NO: 111 - protein, 13 aa, Homo sapiens
AAWDFATWPATEV

SEQ ID NO: 112 - protein, 9 aa, Homo sapiens
SSDVAKIPL

SEQ ID NO: 113 - skipped

SEQ ID NO: 114 - protein, 11 aa, Homo sapiens
SSWDNAGDGHV

SEQ ID NO: 115 - protein, 9 aa, Homo sapiens
SSDVLRPPL

SEQ ID NO: 116 - skipped

SEQ ID NO: 117 - protein, 11 aa, Homo sapiens
SSWDNAGDGHV

SEQ ID NO: 118 - protein, 9 aa, Homo sapiens
SSDVFRPPL

SEQ ID NO: 119 - skipped

SEQ ID NO: 120 - protein, 11 aa, Homo sapiens
SSWDQSGDGHV

SEQ ID NO: 121 - protein, 122 aa, Homo sapiens
QSALTQPASVSGSPGQSITISCTGTSSDVFRPPLVSWYQQHPGKAPKLMIYFASLRPSGVSNRFSGSKSGNTASLTISGLQAEDEADYYCSSWDHTGDGHVFGGGTKLTVLGQPKAAPSVTL

SEQ ID NO: 122 - protein, 119 aa, Homo sapiens
QSVLTQPPSASGTPGQRVTISCSGSSSNIRDSFVNWYQQLPGTAPKLLIYATNIRPSGVPDRFSGSKSGTSASLAISGLQSEDEADYYCASWWPYGTVFGGGTKLTVLGQPKAAPSVTL

SEQ ID NO: 123 - protein, 119 aa, Homo sapiens
QSVLTQPPSASGTPGQRVTISCSGSSSNIRDSFVNWYQQLPGTAPKLLIYATNIRPSGVPDRFSGSKSGTSASLAISGLQSEDEADYYCASWWPFGTVFGGGTKLTVLGQPKAAPSVTL

SEQ ID NO: 124 - protein, 119 aa, Homo sapiens
QSALTQPASVSGSPGQSITISCTGTSSDVVKNNFVSWYQQHPGKAPKLMIYFGSVRPSGVSNRFSGKSGNTASLTISGLQAEDEADYYCSSWDMPALFFGGGTKLTVLGQPKAAPSVTL

SEQ ID NO: 125 - protein, 120 aa, Homo sapiens
QSALTQPASVSGSPGQSITISCTGTSSDVVKNNFVSWYQQHPGKAPKLMIYFGSVRPSGVSNRFSGSKSGNTASLTISGLQAEDEADYYCSSWDEPDRPFGGGTKLTVLGQPKAAPSVTL

SEQ ID NO: 126 - protein, 120 aa, Homo sapiens
QSALTQPASVSGSPGQSITISCTGTSSDVVKNNFVSWYQQHPGKAPKPMIYFGSVRPSGVSNRFSGSKSGNTASLTISGLQAEDEADYYCSSWDLPFLMFGXGTKLTVLGQPKAAPSVTL

SEQ ID NO: 127 - protein, 120 aa, Homo sapiens
QSALTQPASVSGSPGQSITISCTSISSDVVKNNFVSWYQQHPGKAPKLMIYFGSVTADGVSNRFSGSKSGNTASLTISGLQAEDEADYYCSSWDEPDRPFGGGTKLTVLGQPKAAPSVTL

SEQ ID NO: 128 - protein, 120 aa, Homo sapiens
QSALTQPASVSGSPGQSITISCNSPSSDVVKNNFVSWYQQHPGKAPKLMIYFGSVTGPGVSNRFSGSKSGNTASLTISGLQAEDEADYYCSSWDEPDRPFGGGTKLTVLGQPKAAPSVTL

SEQ ID NO: 129 - protein, 123 aa, Homo sapiens
QSVLTQPPSASGTPGQRVTISCSGSSSNIAHKPVNWYQQLPGTAPKLLIYHDNSRPSGVPDRFSGSKSGTSASLAISGLQSEDEADYYCAAWDFATWPATEVFGGGTKLTVLGQPKAAPSVTL

SEQ ID NO: 130 - protein, 123 aa, Homo sapiens
QSVLTQPPSASGTPGQRVTISCSGSSSNIAHKPVNWYQQLPGTAPKLLIYHDNSRPSGVPDRFSGSRSGTSASLAISGLQSEDEADYYCAAWDFSRWPATEVFGGGTKLTVLGQPKAAPSVTL

SEQ ID NO: 131 - protein, 123 aa, Homo sapiens
QSVLTQPPSASGTPGQRVTISCISGSVDIAHKPVNWYQQLPGTAPKLLIYHDTSTPDGVPDRFSGSKSGTSASLAISGLQSEDEADYYCAAWDFATWPATEVFGGGTKLTVLGQPKAAPSVTL

SEQ ID NO: 132 - protein, 122 aa, Homo sapiens
QSALTQPASVSGSPGQSITISCTGTSSDVAKIPLVSWYQQHPGKAPKLMIYFASLRPSGVSNRFSGSKSGNTASLTISGLQAEDEADYYCSSWDNAGDGHVFGGGTKLTVLGQPKAAPSVTL

SEQ ID NO: 133 - protein, 122 aa, Homo sapiens
QSALTQPASVSGSPGQSITISCTGTSSDVLRPPLVSWYQQHPGKAPKLMIYFASLRPSGVSNRFSGSKSGNTASLTISGLQAEDEADYYCSSWDNAGDGHVFGGGTKLTVLGQPKAAPSVTL

SEQ ID NO: 134 - protein, 122 aa, Homo sapiens
QSALTQPASVSGSPGQSITISCTGTSSDVFRPPLVSWYQQHPGKAPKLMIYFASLRPSGVSNRFSGSKSGNTASLTISGLQAEDEADYYCSSWDQSGDGHVFGGGTKLTVLGQPKAAPSVTL

SEQ ID NO: 135 - protein, 214 aa, Homo sapiens
QSVLTQPPSASGTPGQRVTISCSGSSSNIRDSFVNWYQQLPGTAPKLLIYATNIRPSGVPDRFSGSKSGTSASLAISGLQSEDEADYYCAAWHPYYTLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 136 - protein, 214 aa, Homo sapiens
QSVLTQPPSASGTPGQRVTISCSGSSSNIRDSFVNWYQQLPGTAPKLLIYATNIRPSGVPDRFSGSKSGTSASLAISGLQSEDEADYYCASWWPYGTVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 137 - protein, 214 aa, Homo sapiens
QSVLTQPPSASGTPGQRVTISCSGSSSNIRDSFVNWYQQLPGTAPKLLIYATNIRPSGVPDRFSGSKSGTSASLAISGLQSEDEADYYCASWWPFGTVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 138 - protein, 214 aa, Homo sapiens
QSALTQPASVSGSPGQSITISCTGTSSDVVKNNFVSWYQQHPGKAPKLMIYFGSVRPSGVSNRFSGKSGNTASLTISGLQAEDEADYYCSSWDMPALFFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 139 - protein, 215 aa, Homo sapiens
QSALTQPASVSGSPGQSITISCTGTSSDVVKNNFVSWYQQHPGKAPKLMIYFGSVRPSGVSNRFSGSKSGNTASLTISGLQAEDEADYYCSSWDEPDRPFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 140 - protein, 215 aa, Homo sapiens
QSALTQPASVSGSPGQSITISCTGTSSDVVKNNFVSWYQQHPGKAPKPMIYFGSVRPSGVSNRFSGSKSGNTASLTISGLQAEDEADYYCSSWDLPFLMFGXGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 141 - protein, 215 aa, Homo sapiens
QSALTQPASVSGSPGQSITISCTSISSDVVKNNFVSWYQQHPGKAPKLMIYFGSVTADGVSNRFSGSKSGNTASLTISGLQAEDEADYYCSSWDEPDRPFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 142 - protein, 215 aa, Homo sapiens
QSALTQPASVSGSPGQSITISCNSPSSDVVKNNFVSWYQQHPGKAPKLMIYFGSVTGPGVSNRFSGSKSGNTASLTISGLQAEDEADYYCSSWDEPDRPFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 143 - protein, 218 aa, Homo sapiens
QSVLTQPPSASGTPGQRVTISCSGSSSNIAHKPVNWYQQLPGTAPKLLIYHDNSRPSGVPDRFSGSKSGTSASLAISGLQSEDEADYYCAAWDFATWPATEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 144 - protein, 218 aa, Homo sapiens
QSVLTQPPSASGTPGQRVTISCSGSSSNIAHKPVNWYQQLPGTAPKLLIYHDNSRPSGVPDRFSGSRSGTSASLAISGLQSEDEADYYCAAWDFSRWPATEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 145 - protein, 218 aa, Homo sapiens
QSVLTQPPSASGTPGQRVTISCISGSVDIAHKPVNWYQQLPGTAPKLLIYHDTSTPDGVPDRFSGSKSGTSASLAISGLQSEDEADYYCAAWDFATWPATEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 146 - protein, 216 aa, Homo sapiens
QSALTQPASVSGSPGQSITISCTGTSSDVAKIPLVSWYQQHPGKAPKLMIYFASLRPSGVSNRFSGSKSGNTASLTISGLQAEDEADYYCSSWDNAGDGHVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 147 - protein, 217 aa, Homo sapiens
QSALTQPASVSGSPGQSITISCTGTSSDVLRPPLVSWYQQHPGKAPKLMIYFASLRPSGVSNRFSGSKSGNTASLTISGLQAEDEADYYCSSWDNAGDGHVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 148 - protein, 217 aa, Homo sapiens
QSALTQPASVSGSPGQSITISCTGTSSDVFRPPLVSWYQQHPGKAPKLMIYFASLRPSGVSNRFSGSKSGNTASLTISGLQAEDEADYYCSSWDQSGDGHVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 149 - protein, 114 aa, Homo sapiens
DIVMTQTPLSLSVTPGQPASISCKSSGDLLTFEGKTYLYWYLQKPGQPPQLLIYEVSNRFSGVPDRFSGSGSGTDFTLKISRVEAEDVGVYYCMQAHSTRFAFTFGQGTKVEIK

SEQ ID NO: 150 - protein, 111 aa, Homo sapiens
QSVLTQPPSASGTPGQRVTISCSGSSSNIGSNTVNWYQQLPGTAPKLLIYSENTRPSGVPDRFSGSKSGTSASLAISGLQSEDEADYYCAAWDLNPAIGYVFGGGTKLTVL

SEQ ID NO: 151 - protein, 114 aa, Homo sapiens
DIVMTQTPLSLSVTPGQPASISCKSSDDLLRFSGKTYLYWYLQKPGQPPQLLIYEVSNRFSGVPDRFSGSGSGTDFTLKISRVEAEDVGVYYCMQASRSKVAFTFGQGTKVEIK

SEQ ID NO: 152 - protein, 114 aa, Homo sapiens
DIVMTQTPLSLSVTPGQPASISCKSSGDLLSFDGKTYLYWYLQKPGQPPQLLIYEVSNRFSGVPDRFSGSGSGTDFTLKISRVEAEDVGVYYCMQASKHRFGFTFGQGTKVEIK

SEQ ID NO: 153 - protein, 114 aa, Homo sapiens
DIVMTQTPLSLSVTPGQPASISCKSSGDLLEFAGKTYLYWYLQKPGQPPQLLIYEVSNRFSGVPDRFSGSGSGTDFTLKISRVEAEDVGVYYCMQAHGSKIGFTFGQGTKVEIK

SEQ ID NO: 154 - protein, 108 aa, Homo sapiens
DIQMTQSPSTLSASVGDRVTITCRASQEIHSHLSWYQQKPGKAPKLLIYSTSTLESGVPSRFSGSGSGTEFTLTISSLQPDDFATYYCQQGFRFAFSTFGQGTKVEIK

SEQ ID NO: 155 - protein, 113 aa, Homo sapiens
DIVMTQSPDSLGVSLGERATINCKSSQSILYSSSNRNYLAWYQQKPGQPPKLLIYWASTRESGVPDRFSGSGSGTDFTLTISSLQAEDVGVYYCQQNFRPPETFGGGTKVEIK

SEQ ID NO: 156 - protein, 110 aa, Homo sapiens
EIVLTQSPGTLSLSPGERATLSCRASQAVFSLDLHWYQQKPGQAPRLLIYSTSKRATGIPDRFSGSGSGTDFTLTISRLEPEDFAVYYCQQGPAFKPDVTFGQGTKVEIK

SEQ ID NO: 157 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCHQRYRPPETFGQGTKVEIK

SEQ ID NO: 158 - protein, 107 aa, Homo sapiens
DIQMTQSPSSLSASVGDRVTITCRASQSIGAYLNWYQQKPGKAPKLLIYHASDLQSGVPSRFSGSGSGTDFTLTISSLQPEDFATYYCQQYKDRPLTFGQGTKVEIK

SEQ ID NO: 159 - protein, 114 aa, Homo sapiens
DIVMTQTPLSLSVTPGQPASISCKSSGNLLTHDGKTYLYWYLQKPGQPPQLLIYEVSNRFSGVPDRFSGSGSGTDFTLKISRVEAEDVGVYYCMQANALKFGFTFGQGTKVEIK

SEQ ID NO: 160 - protein, 114 aa, Homo sapiens
DIVMTQTPLSLSVTPGQPASISCKSSSDLLRFEGKTYLYWYLQKPGQPPQLLIYEVSNRFSGVPDRFSGSGSGTDFTLKISRVEAEDVGVYYCMQAHASALKFTFGQGTKVEIK

SEQ ID NO: 161 - protein, 114 aa, Homo sapiens
DIVMTQTPLSLSVTPGQPASISCKSSGNLLTHGGKTYLYWYLQKPGQPPQLLIYEVSNRFSGVPDRFSGSGSGTDFTLKISRVEAEDVGVYYCMQAHADGFGFTFGQGTKVEIK

SEQ ID NO: 162 - protein, 114 aa, Homo sapiens
DIVMTQTPLSLSVTPGQPASISCKSSGDLLRFDGKTYLYWYLQKPGQPPQLLIYEVSNRFSGVPDRFSGSGSGTDFTLKISRVEAEDVGVYYCMQASETAVRFTFGQGTKVEIK

SEQ ID NO: 163 - protein, 114 aa, Homo sapiens
DIVMTQTPLSLSVTPGQPASISCKSSTDLLTFEGKTYLYWYLQKPGQPPQLLIYEVSNRFSGVPDRFSGSGSGTDFTLKISRVEAEDVGVYYCMQAHSSKFKFTFGQGTKVEIK

SEQ ID NO: 164 - protein, 114 aa, Homo sapiens
DIVMTQTPLSLSVTPGQPASISCKSSGDLLSTDGKTYLYWYLQKPGQPPQLLIYEVSNRFSGVPDRFSGSGSGTDFTLKISRVEAEDVGVYYCMQASGSRFYFTFGQGTKVEIK

SEQ ID NO: 165 - protein, 113 aa, Homo sapiens
DIVMTQSPDSLAVSLGERAIINCKSSQTLLYKYDNKNYLAWYQHKPGQPPKLLFYWASTRESGVPDRFSGSGSGTDFTLTINSLQAEDVAVYYCHQYLYPAETFGQGTKLEIK

SEQ ID NO: 166 - protein, 221 aa, Homo sapiens
DIVMTQTPLSLSVTPGQPASISCKSSGDLLTFEGKTYLYWYLQKPGQPPQLLIYEVSNRFSGVPDRFSGSGSGTDFTLKISRVEAEDVGVYYCMQAHSTRFAFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 217 aa, Homo sapiens
QSVLTQPPSASGTPGQRVTISCSGSSSNIGSNTVNWYQQLPGTAPKLLIYSENTRPSGVPDRFSGSKSGTSASLAISGLQSEDEADYYCAAWDLNPAIGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 168 - protein, 221 aa, Homo sapiens
DIVMTQTPLSLSVTPGQPASISCKSSDDLLRFSGKTYLYWYLQKPGQPPQLLIYEVSNRFSGVPDRFSGSGSGTDFTLKISRVEAEDVGVYYCMQASRSKVAFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 221 aa, Homo sapiens
DIVMTQTPLSLSVTPGQPASISCKSSGDLLSFDGKTYLYWYLQKPGQPPQLLIYEVSNRFSGVPDRFSGSGSGTDFTLKISRVEAEDVGVYYCMQASKHRFGFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 221 aa, Homo sapiens
DIVMTQTPLSLSVTPGQPASISCKSSGDLLEFAGKTYLYWYLQKPGQPPQLLIYEVSNRFSGVPDRFSGSGSGTDFTLKISRVEAEDVGVYYCMQAHGSKIGFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 215 aa, Homo sapiens
DIQMTQSPSTLSASVGDRVTITCRASQEIHSHLSWYQQKPGKAPKLLIYSTSTLESGVPSRFSGSGSGTEFTLTISSLQPDDFATYYCQQGFRFAFSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 220 aa, Homo sapiens
DIVMTQSPDSLGVSLGERATINCKSSQSILYSSSNRNYLAWYQQKPGQPPKLLIYWASTRESGVPDRFSGSGSGTDFTLTISSLQAEDVGVYYCQQNFRPPETFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 217 aa, Homo sapiens
EIVLTQSPGTLSLSPGERATLSCRASQAVFSLDLHWYQQKPGQAPRLLIYSTSKRATGIPDRFSGSGSGTDFTLTISRLEPEDFAVYYCQQGPAFKPDVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCHQRYRPPETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 214 aa, Homo sapiens
DIQMTQSPSSLSASVGDRVTITCRASQSIGAYLNWYQQKPGKAPKLLIYHASDLQSGVPSRFSGSGSGTDFTLTISSLQPEDFATYYCQQYKDRPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 221 aa, Homo sapiens
DIVMTQTPLSLSVTPGQPASISCKSSGNLLTHDGKTYLYWYLQKPGQPPQLLIYEVSNRFSGVPDRFSGSGSGTDFTLKISRVEAEDVGVYYCMQANALKFGFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 221 aa, Homo sapiens
DIVMTQTPLSLSVTPGQPASISCKSSSDLLRFEGKTYLYWYLQKPGQPPQLLIYEVSNRFSGVPDRFSGSGSGTDFTLKISRVEAEDVGVYYCMQAHASALKFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 221 aa, Homo sapiens
DIVMTQTPLSLSVTPGQPASISCKSSGNLLTHGGKTYLYWYLQKPGQPPQLLIYEVSNRFSGVPDRFSGSGSGTDFTLKISRVEAEDVGVYYCMQAHADGFGFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - protein, 221 aa, Homo sapiens
DIVMTQTPLSLSVTPGQPASISCKSSGDLLRFDGKTYLYWYLQKPGQPPQLLIYEVSNRFSGVPDRFSGSGSGTDFTLKISRVEAEDVGVYYCMQASETAVRFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 221 aa, Homo sapiens
DIVMTQTPLSLSVTPGQPASISCKSSTDLLTFEGKTYLYWYLQKPGQPPQLLIYEVSNRFSGVPDRFSGSGSGTDFTLKISRVEAEDVGVYYCMQAHSSKFKFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 221 aa, Homo sapiens
DIVMTQTPLSLSVTPGQPASISCKSSGDLLSTDGKTYLYWYLQKPGQPPQLLIYEVSNRFSGVPDRFSGSGSGTDFTLKISRVEAEDVGVYYCMQASGSRFYFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 220 aa, Homo sapiens
DIVMTQSPDSLAVSLGERAIINCKSSQTLLYKYDNKNYLAWYQHKPGQPPKLLFYWASTRESGVPDRFSGSGSGTDFTLTINSLQAEDVAVYYCHQYLYPAETFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0254234 A1
(43) Pub. Date: Aug. 1, 2024

(54) PD-L1xCD28 BISPECIFIC ANTIBODIES FOR IMMUNE CHECKPOINT-DEPENDENT T CELL ACTIVATION

(71) Applicant: Novimmune SA, Geneva (CH)

(72) Inventors: Sara MAJOCCHI, Geneva (CH); Valéry MOINE, Sergy (FR); Krzysztof M. MASTERNAK, Mollens (CH); Limin SHANG, Bellevue (CH); Walter FERLIN, Collonges-Sous-Salève (FR)

(21) Appl. No.: 18/491,130

(22) Filed: Oct. 20, 2023

Related U.S. Application Data
(60) Provisional application No. 63/446,987, filed on Feb. 20, 2023.
(60) Provisional application No. 63/418,264, filed on Oct. 21, 2022.

(51) Int. Cl. C07K 16/28; A61K 39/00; A61P 35/00

(52) U.S. Cl. CPC C07K 16/2827; A61P 35/00; C07K 16/2818; A61K 2039/505; C07K 2317/31; C07K 2317/52; C07K 2317/565; C07K 2317/73; C07K 2317/76

(57) ABSTRACT

The present invention relates to PD-L1xCD28 bispecific antibodies which act as immune checkpoint inhibitors by binding to and blocking PD-L1 (thus preventing its engagement with PD-1 expressed on T cells) that can further deliver a costimulatory signal to T cells by clustering and agonistically engaging CD28.

### CROSS-REFERENCE TO RELATED APPLICATIONS

This application claims the benefit of and priority to U.S. Provisional Application No. 63/418,264, filed on Oct. 21, 2022, and U.S. Provisional Application No. 63/446,987, filed on Feb. 20, 2023, each of which is incorporated herein by reference in its entirety.

### FIELD OF THE INVENTION

The present invention relates fully human PD-L1xCD28 bispecific antibodies (bsAb) in the κλ body format capable of blocking the PD-1/PD-L1 interaction while providing costimulatory T cell signal 2.

### REFERENCE TO AN ELECTRONIC SEQUENCE LISTING

The contents of the electronic sequence listing (NOVI_052_001US_SeqList_ST26.xml; Size: 148,790 bytes; and Date of Creation: Oct. 12, 2023) are herein incorporated by reference in its entirety.

### BACKGROUND OF THE INVENTION

In the past years, novel approaches to stimulate the body's own immune cells to better attack and kill cancer cells have been developed. Examples of successful cancer immunotherapies are monoclonal antibodies capable of blocking so-called immune checkpoints. Currently approved immune checkpoint inhibitors (II) block CTLA-4 (e.g., Ipilimumab, sold under the brand name Yervoy), PD-1 (e.g., Pembrolizumab, sold under the brand name Keytruda and Cemiplimab, sold under the brand name Libtayo) and PD-L1 (e.g., Atezolizumab, sold under the brand name Tecentriq). Durable anti-tumor responses can be obtained in a range of cancer types using ICIs. Unfortunately, responses are limited to a patient subset, and many cancer types are known to be intrinsically resistant to ICI monotherapies.

Other approved cancer immunotherapies include T cell bispecific antibodies—bridging T cells to target cells expressing a tumor associated antigen (TAA) via the CD3 receptor on T cells—and Chimeric Antigen Receptor (CAR) T cells. Despite the very good anti-tumor responses observed with treatments based on T cell bispecifics or CAR T cells in hematological malignancies, so far there has been no real breakthrough of these approaches in the context of solid cancers, leaving many cancer patients with no therapeutic options.

T cell costimulatory bispecific antibodies are a novel class of therapeutics which can elicit anti-tumor response, especially in combination with T cell bispecific antibodies or immune checkpoint inhibitors (ICIs). Preclinical studies have shown the benefit of adding costimulatory CD28 bsAbs for the treatment of solid tumors, enhancing the efficacy of bispecific T cell engagers (Correnti et al. 2018; Skokos et al. 2020) or PD-(L)1 checkpoint inhibitors (Waite et al. 2020). They act by providing costimulatory signal 2 to T cells within the tumor microenvironment. The specificity of CD28 costimulatory bsAbs is given by a targeting anti-tumor-associated antigen (TAA) arm, which is paired with a so-called effector arm, and notably an agonist anti-CD28 arm. To date, several TAAxCD28 bsAbs have been described (by Correnti et al. 2018, but also in WO2019246514, WO2020132066, WO2020198009, WO2020127618, WO2020132024, WO2021259890 and WO2022040482), with some of them being actively tested in early-phase clinical trials (ClinicalTrials.gov Identifiers NCT03972657, NCT04590326, NCT04626635, NCT05219513 or NCT05585034).

To further enhance the anti-tumor activity of CD28 bsAbs, the anti-TAA targeting arm could be replaced by an antibody arm that has an intrinsic therapeutic activity. As shown in FIG. 1, the use of an anti-PD-L1 arm presents multiple advantage, because: (1) the monovalent blockade of PD-L1 still prevents PD-1 engagement on T cells; (2) PD-L1 expressed on tumor, immune or stromal cells inhibits T cells via PD-1 engagement, which negatively regulates CD28 co-stimulatory signal; (3) PD-L1 expressed on APCs sequesters CD80 and prevents it from co-stimulating CD28 on T cells.

Such PD-L1xCD28 bsAb can (1) provide costimulatory signal 2 when bridging PD-L1 (tumor) cells and T cells, while preventing the PD-1/PD-L1 interaction; (2) prevent PD-L1 from APCs from interacting with PD-1 on T cells, in the tumor microenvironment but also in the draining lymph nodes; (3) prevent PD-L1 from DC from sequestering CD80, thus favoring the CD80/CD28 interaction; (4) prevent PD-L1 on stromal and immune cells from interacting with PD-1 on T cells.

Accordingly, there is a need for novel antibodies and therapeutics that enable dual targeting of CD28 and PD-11.

### SUMMARY OF THE INVENTION

The invention provides immune checkpoint driven costimulatory bispecific antibodies. In some aspects, the antibody is a bispecific antibody that has a first antigen binding domain that binds to PD-L1; and a second binding domain that binds CD28, referred to herein as PD-L1xCD28 bsAbs.

The PD-L1xCD28 bsAbs have a common heavy chain having a complementarity determining region 1 (CDR1) comprising the amino acid sequence of (SEQ ID NO: 6); a complementarity determining region 2 (CDR2) comprising the amino acid sequence of (SEQ ID NO: 7); and a complementarity determining region 3 (CDR3) comprising the amino acid sequence of (SEQ ID NO:8).

In some aspects PD-L1xCD28 bsAbs have a first light chain variable region having: a CDR1 comprising the amino acid sequence of SEQ ID NO: 13; a CDR2 comprising the amino acid sequence of SEQ ID NO: 14; and a CDR3 comprising the amino acid sequence of SEQ ID NO: 15 [S79];

In some aspects PD-L1xCD28 bsAbs have a second light chain variable region having: a CDR1 comprising the amino acid sequence of SEQ ID NO: 18; a CDR2 comprising the amino acid sequence of SEQ ID NO: 19; and a CDR3 comprising the amino acid sequence of SEQ ID NO: 20 [AI3]; or a CDR1 comprising the amino acid sequence of SEQ ID NO: 23; a CDR2 comprising the amino acid sequence of SEQ HD NO: 24; and a CDR3 comprising the amino acid sequence of SEQ ID NO: 25 [AI13];

In some aspects the first and second heavy chain variable region comprises the amino acid sequence of SEQ ID NO: 10. In some aspects the first and second heavy chain comprises the amino acid sequence of SEQ ID NO: 11 or SEQ ID NO: 12.

In some aspects the first light chain variable region of: SEQ ID NO: 16.

In other aspects the first light chain has the amino acid sequence of: SEQ ID NO: 17.

In some aspects the bispecific antibody has a second light chain variable region of SEQ ID NO: 21; SEQ ID NO: 26.

In other aspects the bispecific antibody has a second light chain of SEQ NO: 22; SEQ ID NO: 27.

Also included in the invention are compositions of any of the bispecific antibodies disclosed herein. Optionally the composition further comprises a CD3xCEA bispecific antibody having two identical heavy chains comprising the amino acid sequence of SEQ ID NO: 3, a first light chain having the amino acid sequence of SEQ ID NO: 4, and a second light chain having the amino acid sequence of SEQ ID NO: 5.

In some aspects the bispecific antibody of has a first light chain that is a kappa and a second light chain that is a lambda.

In another aspect the bispecific antibody has a first light chain that is a lambda and the second light chain that is a kappa.

In some embodiments, a portion of the first light chain is of the kappa type and at least a portion of the second light chain is of the lambda type. In some embodiments, the first light chain comprises at least a Kappa constant region. In some embodiments, the first light chain further comprises a Kappa variable region. In some embodiments, the first light chain further comprises a Lambda variable region.

In some embodiments, the second light chain comprises at least a Lambda constant region. In some embodiments, the second light chain further comprises a Lambda variable region. In some embodiments, the second light chain further comprises a Kappa variable region.

In some embodiments the first light chain comprises a Kappa constant region and a Kappa variable region, and wherein the second light chain comprises a Lambda constant region and a Lambda variable region.

Optionally, the bispecific antibody has an Fc domain comprising one or more amino acid substitutions that reduce binding to an activating Fc receptor and/or reduce effector function.

For example, the bispecific antibody has a L234A and L235A substitution. Additionally, the bispecific antibody has a P329A, P329G or P329R substitution.

The bispecific antibody has an IgG isotype. The bispecific antibody is a human antibody. The bispecific antibody enables PD-L1-dependent T cell activation. In some embodiments, the immune stimulation by the bispecific antibody occurs inside and/or at the tumor. In some embodiments, the immune stimulation by the bispecific antibody occurs outside the tumor. In some embodiments, the immune stimulation by the bispecific antibody occurs in the lymphoid organs or system.

The disclosure provides a composition comprising the bispecific antibody described herein and a pharmaceutically acceptable carrier.

The disclosure provides a method of reducing the proliferation of and/or enhancing the killing a tumor cell comprising contacting the cell with the composition comprising the bispecific antibody described herein. The disclosure provides a method of treating a cancer in a subject comprising administering to the subject the composition comprising the bispecific antibody described herein.

In some aspects, the disclosure provides the use of a composition comprising the bispecific antibody described herein for treating, preventing, or delaying the progression of pathologies. In some embodiments, the pathology is cancer. In some embodiments, the cancer is a solid tumor. In some embodiments, the solid tumor is or is derived from breast cancer, ovarian cancer, head and neck cancer, bladder cancer, melanoma, mesothelioma, colorectal cancer, cholangiocarcinoma, pancreatic cancer, lung cancer, leiomyoma, leiomyosarcomna, kidney cancer, glioma, glioblastoma, endometrial cancer, esophageal cancer, biliary gastric cancer, prostate cancer, or combinations thereof.

The invention further comprises an antibody having an antigen binding domain that binds to CD28; wherein the antigen binding domain has a heavy chain variable region having a complementarity determining region 1 (CDR1) comprising the amino acid sequence of (SEQ ID NO: 6); a complementarity determining region 2 (CDR2) comprising the amino acid sequence of (SEQ ID NO: 7); and a complementarity determining region 3 (CDR3) comprising the amino acid sequence of (SEQ ID NO:8); and a light chain variable region having: a CDR1 comprising the amino acid sequence of SEQ ID NO: 18; a CDR2 comprising the amino acid sequence of SEQ ID NO: 19; and a CDR3 comprising the amino acid sequence of SEQ ID NO: 20; [AI3]; or a CDR1 comprising the amino acid sequence of SEQ ID NO: 23; a CDR2 comprising the amino acid sequence of SEQ ID NO: 24; and a CDR3 comprising the amino acid sequence of SEQ ID NO:25; [AI13].

The antibody is a F(ab) fragment, a F(ab′)2 fragment, and Fv fragment or a single chain Fv fragment. The antibody is monospecific. The antibody is monovalent.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1. Mechanisms of action enabled by PD-L1xCD28 bsAbs.

FIG. 2A-C. Concentration dependent binding of AI3S79/N to PD-L1 expressing HPAC cells, pre-activated with IFNγ (A), to CD28 expressing Jurkat cells (B) and CD28 and PD-L1 double negative TIB153 cells (C).

FIG. 3. Concentration dependent blockade of PD-1/PD-L1 interaction as measured by a reporter cell bioassay. The stronger is the blockade of the PD-1/PD-L1 axis, the higher the luminescence of the reporter cells, AI3S79/N blocks the PD-1/PD-L1 axis and thanks to its agonist CD28 arm it also boosts the reporter system, resulting in higher RLU than simple PD-L1 blockers.

FIG. 4. T-cell dependent cellular cytotoxicity of HPAC cells induced by AI3S79/N when combined with a CEAxCD3 bsAb. Effector cells (healthy donor PBMCs) were co-cultured for 6 days with PDL1/CEA double-positive HPAC cells at different E:T ratios (10:1, 3:1, 1:1 and 1:3, from left to right). AI3S79/N synergizes with CEAxCD3 to kill the HPAC cells. No killing is induced in the absence of signal 1 (no CEAxCD3 bsAb added). Dose range of CEAxCD3 with a constant concentration of CD28 bsAb (2.5 μg/mL).

FIG. 5. Upregulation of the T cell activation marker CD25 on human CD4-+ and CD8+ T cells (top and bottom row, respectively) harvested at the end of the TDCC experiment shown on FIG. 4. The combination of CEAxCD3 and PD-L1xCD28 bsAbs induces a stronger activation of both CD4+ and CD8+ T cells than the CEAxCD3 monotherapy.

FIG. 6. Proliferation of human CD4+ and CD8+ T cells (top and bottom row, respectively) harvested at the end of the TDCC experiment shown on FIG. 4. The combination of CEAxCD3 and PD-L1xCD28 bsAbs strongly increases the percentage of proliferative CD4+ and CD8+ T cells compared to CEAxCD3 monotherapy.

FIG. 7A-F. In vivo efficacy study of AI3S79/N in the treatment of subcutaneous syngeneic colorectal cancer model MC38-hPDL1 in CD28 HuGEMM mice. (A) The experimental design of the in in vivo efficacy study. (B) The average tumor volume of the different treatment groups. (C) Tumor volume comparison at study termination (day 28 post treatment initiation). Tumor volume was set to 3000 mm³ for all tumors that grew past 3000 mm³. (D), E and F) Tumor growth of individual mice belonging to the vehicle, Atezolizumab or AI3S79/N group, respectively. Contrary to Atezolizumab, which on average only delayed tumor growth, AI3S79/N as single agent led to tumor regression in all mice, with 6 out of 10 mice marked as tumor-free at study termination.

FIG. 8A-B. (A) Mouse body weight (with tumor correction) following treatment with AI3S79/N according to the experimental treatment plan of FIG. 7A. (B) Mouse cytokine secretion 6 hours post 1^{st} injection following treatment with AI3S79/N according to the experimental treatment plan of FIG. 7A. AI3S79/N was well tolerated in immunocompetent huCD28 mice.

FIG. 9. Average tumor volume of mice that were re-challenged. Tumor rechallenge of surviving mice from the experiment shown in FIG. 7A, with WT MC38 cells on the opposite flank. Rechallenged=surviving mice (n=8), untreated. Naïve mice (n=8), untreated. AI3S79/N induced immunological memory against MC38.

FIG. 10A-B. IL-2 release by fresh or high-density precultured PBMCs isolated from three donors following treatment with TGN1412 (A) or AI3S79/N (B). Contrary to TGN1412, A3S79/N is not superagonist.

FIG. 11A-B. (A.) Scheme of the established T cell and DC mixed lymphocyte reaction (MLR). (B) IL-2 release induced by PD-(L)1 blocking antibodies, including AI3S79/N and anti-PD-(L)1 blocking antibodies such as nivolumab and atezolizumab.

FIG. 12. Body weight (as percentage of Day 0) of irradiated and PBMC engrafted mice treated with TGN1412 or a dose response of AI3S79/N. AI3S79/N was well tolerated in a mouse model sensitive to CD28-mediated CRS.

FIG. 13. Proliferation of CD4+ and CD8+ T cells cultured in plates coated with wet-coating or dry-coating procedures. Each dot represents the average proliferation obtained with a healthy PBMC donor. Contrary to TGN1412, AI3S79/N is not superagonist.

FIG. 14A-B. (A) Killing of NLV-loaded tumor cells and (B) corresponding expansion of NLV-specific T cells (right) induced by a dose-response of A3S79/N. Two donors are shown. A3S79/N induced the killing of NLV-loaded tumor cells and the expansion of NLV-specific T cells in a dose-dependent manner.

FIG. 15A-C. In vivo efficacy study of AI3S79/N in combination with a CEAxCD3 TCE in the treatment of HPAC tumors subcutaneously engrafted in PBMC-humanized NOG mice. (A) The experimental design of the in vivo efficacy study. (B) The average tumor volume of the different treatment groups. (C) Tumor growth of individual mice. Since two PBMC donors (D415 and D417) were used for humanization, mice are clearly identified as being humanized with D415 (dotted line) or D417 (full line). AI3S79/N synergized with a TCE to induce TV regression or stasis.

FIG. 16A-B. In vivo efficacy study of AI3S79/N in combination with a CEAxCD3 TCE in the treatment of HPAF-II subcutaneous tumors in fully humanized BRGSF-HIS mice. (A) The average tumor volume of the different treatment groups. (B) Tumor growth of individual mice. Asterisks (*) denote animals euthanized because of tumor ulceration (instead of tumor volume endpoint). AI3S79/N synergized with a TCE to reduce tumor progression.

FIG. 17. AI3S79/N concentrations after single (at 0.5 or 10 mg/kg) and repeated (2×10 mg/kg) IV administration in monkeys (semi-logarithmic scale). Black dashed line represents the LLOQ value (0.02 μg/mL).

FIG. 18. Serum cytokine concentrations in cynomolgus monkeys following single or repeat-dosing (IV) with AI3S79/N. AUS79/N administration resulted only in mild and transient IL-6 release.

FIG. 19. CRP levels in animals administered with a single or repeated (n=2) injections of AI3S79/N. AI3S79/N treatment induced mild and transient CRP release.

### DETAILED DESCRIPTION OF THE INVENTION

The invention is based on a bispecific antibody (bsAb) capable of immune checkpoint-dependent T cell activation and tumor cell killing. Specifically, the invention is based upon bsAb co-engagement of the immune checkpoint PD-L1 expressed, among others, at the surface of tumor cells to mediate CD28 clustering and thus PD-L1-mediated T cell activation.

The bsAbs of the invention are characterized by a single agonist CD28 antigen binding domain for the monovalent co-stimulation of CD28, and a second antigen binding domain capable of binding specifically and monovalently to PD-L1, which prevent PD-L1 from engaging PD-1 expressed on T cells.

### CD28

CD28 is a key co-stimulatory receptor expressed at the surface of T lymphocyte. It belongs to a subfamily of costimulatory molecules characterized by an extracellular variable immunoglobulin-like domain. Other members in the family of molecules include CTLA-4, ICOS, PD-1 and BTLA.

In humans, CD28 is expressed at the cell surface of T lymphocyte as a disulfide-linked homodimer, and is found on approximately 80% of human CD4+ T cells and 50% of CD8+ T cells.

Despite lacking intrinsic enzymatic activity, CD28 engagement by its ligands leads to specific phosphorylation and transcriptional signaling which ultimately results in metabolic changes and in the production of key cytokines, chemokines, and survival signals that are essential for long-term expansion and differentiation of T cells.

The primary ligands for CD28 are CD80 (B71) and CD86 (137.2), which are mainly expressed at the surface of professional antigen presenting cells (APC). CD80 and CD86 diverge in their expression patterns, multimeric states, and functionality. Because (CD28 and CTLA-4 are highly homologous, they compete for the same ligands. However, since CTLA-4 binds these ligands with a higher affinity than CD28, CTLA-4 competes with CD28 for ligands and ultimately suppresses T cells responses.

Several anti-CD28 monoclonal antibodies have been proposed for the therapeutic targeting of CD28. A fraction of the identified anti-CD28 antibodies, termed superagonist (SA) antibodies, were found to induce the full activation of primary resting T cells even in the absence of TCR ligation (signal 1), via the clustering of CD28 at the surface of the T cells. The first-in-human study of one of such SA anti-CD28 antibodies, TGN1412, resulted however in severe inflammatory reactions as well as chronic organ failure in all healthy volunteers subjected to the treatment. A cytokine storm predicted by neither in vivo nor in vitro preclinical safety studies was the cause of these adverse events.

To minimize the side effect of anti-CD28 monoclonal antibodies, B7×anti-tumor-associated antigen (TAA) fusion proteins were proposed by Holliger et al. (Holliger et al. 1999). These were found to be equally effective but more specific compared with anti-CD28 monoclonal antibodies, i.e., a B7×anti-CEA bispecific fusion protein can only activate T cells in the presence of CEA-expressing cells.

On the same principle, CD28 bsAbs cannot cluster CD28 at the surface of T cells on their own but require the engagement of a second target at the surface of another cell. As such, CD28 bispecific antibodies are unable to costimulate T cells on their own.

In the context of this invention, agonist anti-CD28 binding domains are paired to an anti-PD-L1 binding domain, resulting in molecules capable of bridging T cells to cells expressing PD-L1.

Furthermore, even in presence of PD-L1-positive cells which allow for CD28 clustering at the surface of the T cells, the full cytotoxic potential of T cells can only be unleashed in presence of primary T cell stimulation via the TCR. This contrasts with the bivalent superagonist CD28 monoclonal antibodies that were described above.

Preclinical studies have shown the benefit of adding costimulatory tumor associated antigenxCD28 (TAAxCD28) bsAbs for the treatment of solid tumors, boosting the efficacy of bispecific T cell engagers or PD-(L)1 checkpoint inhibitors. Examples of agonist TAAxCD28 bsAbs are described in WO2019246514, WO2020198009, WO2020132066, WO2020132024, WO2020127618, WO2021259890, WO2021155071 and WO2022040482, with some of these molecules currently being tested in clinical trials (ClinicalTrials.gov Identifiers: NCT04590326, NCT03972657, NCT04626635, NCT05219513, NCT05585034).

### PD-L1

Programmed cell death ligand-1 (PD-L1), also referred to as B7-H1 and CD274, is a transmembrane protein constitutively expressed on both hematopoietic and non-hematopoietic healthy tissues. It can also be expressed on tumor cells and tumor stroma. In cancer, the expression of the inhibitory receptor PD-1 is considered as a hallmark of exhausted T cells, which exhibit a dysfunctional phenotype due to persistent antigenic and inflammatory stimulation. Furthermore, it has been shown that upregulation of PD-L1 in the tumor microenvironment allows tumors to evade the host immune system, by interacting with PD-1 on T cells. Multiple studies have reported that PD-L1 is expressed in a variety of tumor tissues, either on tumor cells or immune-infiltrating cells or on both. In patients, blocking the interaction of PD-1 with PD-L1 using monoclonal antibodies has proved to be a successful therapy in a range of cancer indications and is widely thought to enhance antitumor T-cell responses by reversing or preventing the onset of T cell exhaustion, but also by promoting the expansion of T cells during T cell priming in the tumor draining lymph nodes. However, despite the considerable improvement in patient outcome that has been achieved with PD-1/PD-L1 checkpoint inhibitors, durable responses to these therapies are observed in only a minority of patients, and intrinsic or acquired resistances are common.

### Bispecific Antibodies

The bsAbs antibodies according to the invention may be generated de novo or may be engineered from existing monospecific CD28 antibodies and PD-L1 antibodies.

The bsAbs of the invention can be based on any of the different antibody formats that have been previously described. In general, IgG-like formats are preferred as they provide favorable properties such as long half-life and potentially reduced immunogenicity, but any other molecular bispecific format can also be used for the invention

The heavy and light chain amino acid sequences of the antibodies identified by their United States Adopted Names (USAN are available for example via the American Medical Association at https://wwv.ama-assn.org/or via the CAS registry).

Monospecific CD28 and PD1-L1 binding variable domains may be selected de novo from for example a phage display library, where the phage is engineered to express human immunoglobulins or portions thereof such as Fabs, single chain variable fragments (scFv), or unpaired or paired antibody variable regions and subsequently engineered into a bispecific format. The CD28 and PD-L1 variable domains can be isolated for example from phage display libraries expressing antibody heavy and light chain variable regions as fusion proteins at the surface of bacteriophage M13, fused to the capside protein pIII.

The antibody libraries are screened for binding CD28 antibodies and PD-L1 and the obtained positive clones are further characterized. Such phage display methods for isolating human antibodies are established in the art. See for example: U.S. Pat. Nos. 5,223,409; 5,403,484; and 5,571,698, 5,427,908, 5,580,717, 5,969,108, 6,172,197, 5,885,793; 6,521,404; 6,544,731; 6,555,313; 6,582,915 and 6,593,081. The obtained de novo variable regions binding are engineered to bispecific formats using the methods know in the art and described herein.

Additionally, bispecific antibodies of the invention can be made using the techniques, including those disclosed in WO 2012/023053, filed Aug. 16, 2011, the contents of which are hereby incorporated by reference in their entirety. The methods described in WO 2012/023053 generate bispecific antibodies that are identical in structure to a human immunoglobulin. This type of molecule is composed of two copies of a unique heavy chain polypeptide, a first light chain variable region fused to a constant Kappa domain and second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions.

However, it is also possible to obtain bispecific antibodies of the invention by fusing a Kappa light chain variable domain to a constant Lambda domain for a first specificity and fusing a Lambda light chain variable domain to a constant Kappa domain for the second specificity. The bispecific antibodies described in WO 2012/023053 are referred to as IgGκλ antibodies or “κλ bodies,” a new fully human bispecific IgG format. This κλ-body format allows the affinity purification of a bispecific antibody that is undistinguishable from a standard IgG molecule with characteristics that are undistinguishable from a standard monoclonal antibody and, therefore, favorable as compared to previous formats.

In addition to methods described above, bispecific antibodies of the invention can be generated in vitro in a cell-free environment by introducing asymmetrical mutations in the CH3 regions of two monospecific homodimeric antibodies and forming the bispecific heterodimeric antibody from two parent nonspecific homodinmeric antibodies in reducing conditions to allow disulfide bond isomerization according to methods described in Intl. Pat. Publ. No. WO2011/131746. In the methods, the first monospecific bivalent antibody and the second monospecific bivalent antibody are engineered to have certain substitutions at the CH3 domain that promoter heterodimer stability; the antibodies are incubated together under reducing conditions sufficient to allow the cysteines in the hinge region to undergo disulfide bond isomerization; thereby generating the bispecific antibody by Fab arm exchange.

Antibodies of the present invention have two or more antigen binding domains and are bispecific. Bispecific antibodies of the invention include antibodies having a full length antibody structure or partial length antibody structure such as Fab

“Full length antibody” as used herein refers to an antibody having two full length antibody heavy chains and two full length antibody light chains. A full length antibody heavy chain (HC) consists of well-known heavy chain variable and constant domains VH, CH, CH2, and CH3. A full-length antibody light chain (LC) consists of well-known light chain variable and constant domains VL and CL. The full-length antibody may be lacking the C-terminal lysine (K) in either one or both heavy chains.

The term “Tab-arm” or “half molecule” refers to one heavy chain-light chain pair that specifically binds an antigen.

Full length bispecific antibodies of the invention may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope.

“Homodimerization” as used herein refers to an interaction of two heavy chains having identical CH3 amino acid sequences. “Homodimer” as used herein refers to an antibody having two heavy chains with identical CR3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Intl. Publ. No. WO 2006/028936) may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S L368A Y407V.

Other strategies such as promoting heavy chain heterodimerization using electrostatic interactions by substituting positively charged residues at one CH-3 surface and negatively charged residues at a second CH3 surface may be used, as described in US Pat. Publ. No. US2010/0015133; US Pat. Publ. No. US2009/0182127; US Pat. Publ, No. US2010/028637 or US Pat. Publ. No. US2011/0123532. In other strategies, heterodimerization may be promoted by following substitutions (expressed as modified position in the first CH3 domain of the first heavy chain/modified position in the second CH3 domain of the second heavy chain): L351Y_F405A-Y407V/T394W, T3661_K392M_T394W/F405A Y407V, T366L_K392M_T394W/F405A Y407V, L351Y_Y407A/T366A_K409F, L351Y_Y407A/T366V_K409F, Y407A/T366A_K409F, or T350V L351Y_F405A Y407V/T350V_T366L_K392L_T394W as described in U.S. Pat. Publ. No. US2012/0149876 or U.S. Pat. Publ. No. US2013/0195849

Exemplary anti-cell surface antibodies that may be used to engineer bispecific molecules include for example anti-tumor associate antigen antibodies know in the art, such as Pertuzumab and Trastuzurnab (HER-2); Cetuxirnab, Necitumumab, Panitumumab and Amivantamab (EGFR); Labetuzumab and Cibisatamab (CEA); Amatuximab (mesothelin); Cordrituzumab (glypican 3); Atezolizuinab, Aveluinab and Durvalumab (PD-L11); Blinatumomab (CD19); Brentuximab (CD30); Daratumumab (CD38); Gemtuzumab (CD33); Tositumomab 9CD22) or Obinutuzumab, Ocrelizumab, Ofatumumab, Rituximab, and Ibriturmomab (CD20).

Exemplary Bispecific Antibodies that Bind to CD28 and Pd-L1

The bispecific antibodies of the invention have one antigen binding region that is specific for PD-L1 and a second antigen binding region that is specific for CD28. But another way the bispecific antibodies are monovalent for PD-1 and CD28. The bispecific antibodies share a common heavy chain.

In some embodiments, the heavy chains are native heavy chains (i.e., does not contain any mutations). In some embodiments, the heavy chains comprise mutations relative to the native heavy chain. In some embodiments, the heavy chains are of the IgG14 type containing different mutations to minimize effector functions. Optionally, the bispecific antibodies have light chains of different types. For example, one light chain is a kappa and the other light chain is a lambda light chain (i.e., kl-body). Differing light chains allows the bispecific to be purified easily using kappa and lambda select resins.

Exemplary PD-L1 antibodies from which the PD-L1 antigen binding region can be derived from include the S8 antibody, the S9 antibody, the S37 antibody, the S14 antibody, the S15 antibody, the S17 antibody, the S57 antibody, the S58 antibody, the S28 antibody, the S30 antibody, the S94 antibody, the S23 antibody, the S46 antibody, the S71 antibody, and the S79 antibody. Exemplary CD28 antibodies from which the CD28 antigen binding region can be derived from include the AI3 antibody, the AI13 antibody, the AI5 antibody, the AI7 antibody, the AI8 antibody, the A19 antibody, the AI10 antibody, the AI11 antibody, the AI12 antibody, the AI14 antibody, the AI15 antibody, the AI16 antibody, the AI17 antibody, the AI18 antibody, the AI19 antibody, the A120 antibody, the AI21 antibody, the AI22 antibody, and the A123 antibody. Accordingly, reference to antibodies can be written as, for example, “S79xAI3” or “AI3xS79” or “AI3S79” or “S79AI3” to identify a first antigen binding domain and a second antigen binding domain.

In some embodiments, the S79xA3 bispecific antibody has a heavy chain having a complementarity determining region 1 (CDR1) comprising the amino acid sequence of (SEQ ID NO: 6); a complementarity determining region 2 (CDR2) comprising the amino acid sequence of (SEQ FD NO: 7); and a complementarity determining region 3 (CDR3) comprising the amino acid sequence of (SEQ ID NO:8), a lambda light chain variable region having: a CDR1 comprising the amino acid sequence of SEQ ID NO: 13; a. CDR2 comprising the amino acid sequence of SEQ ID NO: 14; and a CDR3 comprising the amino acid sequence of SEQ ID NO: 15, and a kappa light chain having: a CDR1 comprising the amino acid sequence of SEQ ID NO: 18; a CDR2 comprising the amino acid sequence of SEQ ID NO: 19; and a CDR3 comprising the amino acid sequence of SEQ ID NO: 20.

In some embodiments, the S79xAI3 bispecific antibody has a heavy chain variable region comprising the amino acid sequence of SEQ ID NO: 9, a lambda light chain variable region comprising the amino acid sequence of SEQ ID NO: 16, and a kappa light chain variable region comprising the amino acid sequence of SEQ ID NO: 21.

In some embodiments, the S79xAI3 bispecific antibody has a heavy chain variable and constant region comprising the amino acid sequence selected from SEQ ID NO: 10, SEQ ID NO: 11, or SEQ ID NO: 12, a lambda light chain comprising the amino acid sequence of SEQ ID NO: 17, and a kappa light chain comprising the amino acid sequence of SEQ ID NO: 22.

In some embodiments, the S79 x AI13 bispecific antibody has a heavy chain having a complementarity determining region 1 (CDR1) comprising the amino acid sequence of (SEQ ID NO: 6); a complementarity determining region 2 (CDR2) comprising the amino acid sequence of (SEQ ID NO: 7); and a complementarity determining region 3 (CDR3) comprising the amino acid sequence of (SEQ ID NO:8), a lambda light chain variable region having: a CDR1 comprising the amino acid sequence of SEQ ID NO: 13; a. CDR2 comprising the amino acid sequence of SEQ ID NO: 14; and a CDR3 comprising the amino acid sequence of SEQ ID NO: 15, and a kappa light chain having: a CDR1 comprising the amino acid sequence of SEQ ID NO: 23; a CDR2 comprising the amino acid sequence of SEQ ID NO: 24; and a CDR3 comprising the amino acid sequence of SEQ ID NO: 25.

In some embodiments, the S79 x AI13 bispecific antibody has a heavy chain variable region comprising the amino acid sequence of SEQ ID NO: 9, a lambda light chain variable region comprising the amino acid sequence of SEQ ID NO: 16, and a kappa light chain variable region comprising the amino acid sequence of SEQ ID NO: 26.

In some embodiments, the S79 x AI13 bispecific antibody has a heavy chain variable and constant region comprising the amino acid sequence selected from SEQ ID NO: 10, SEQ ID NO: II, or SEQ ID NO:12, a lambda light chain comprising the amino acid sequence of SEQ ID NO: 17, and a kappa light chain comprising the amino acid sequence of SEQ ID NO: 27.

TABLE 1
Exemplary Amino Acid Sequences of the Invention

| SEQ ID | | |
|---|---|---|
| NO | Description | Amino Acid Sequence |
| SEQ ID | TGN1412 heavy | QVQLVQSGAEVKKPGASVKVSCKASGYTFTSYYIHW |
| NO: 1 | chain | VRQAPGQGLEWIGCIYPGNVNTNYNEKFKDRATLTV |
| | | DTSISTAYMELSRLRSDDTAVYFCTRSHYGLDWNFDV |
| | | WGQGTTVTVSSASTKGPSVFPLAPCSRSTSESTAALGC |
| | | LVKDYFPEPVTVSWNSGALTSGVHTFPAVLQSSGLYS |
| | | LSSVVTVPSSSLGTKTYTCNVDHKPSNTKVDKRVESK |
| | | YGPPCPSCPAPEFLGGPSVFLFPPKPKDTLMISRTPEVT |
| | | CVVVDVSQEDPEVQFNWYVDGVEVHNAKTKPREEQ |
| | | FNSTYRVVSVLTVLHQDWLNGKEYKCKVSNKGLPSSI |
| | | EKTISKAKGQPREPQVYTLPPSQEEMTKNQVSLTCLV |
| | | KGFYPSDIAVEWESNGQPENNYKTTPPVLDSDGSFFL |
| | | YSRLTVDKSRWQEGNVFSCSVMHEALHNHYTQKSLS |
| | | LSLGK |
| SEQ ID | TGN1412 light | DIQMTQSPSSLSASVGDRVTITCHASQNIYVWLNWYQ |
| NO: 2 | chain | QKPGKAPKLLIYKASNLHTGVPSRFSGSGSGTDFTLTI |
| | | SSLQPEDFATYYCQQGQTYPYTFGGGTKVEIKRTVAA |
| | | PSVFIFPPSDEQLKSGTASVVCLLNNFYPREAKVQWK |
| | | VDNALQSGNSQESVTEQDSKDSTYSLSSTLTLSKADY |
| | | EKHKVYACEVTHQGLSSPVTKSFNRGEC |
| SEQ ID | CEAxCD3 common | EVQLVESGGGLVQPGGSLKLSCAASGFTFNTYAMNW |
| NO: 3 | heavy chain | VRQAPGKGLEWVGRIRSKYNNYATYYADSVKDRFTI |
| | | SRDDSKNTAYLQMNSLKTEDTAVYYCVRHGNFGNSY |
| | | VSWFAYWGQGTLVTVSSASTKGPSVFPLAPSSKSTSG |
| | | GTAALGCLVKDYFPEPVTVSWNSGALTSGVHTFPAVL |
| | | QSSGLYSLSSVVTVPSSSLGTQTYICNVNHKPSNTKVD |
| | | KRVEPKSCDKTHTCPPCPAPEAAGGPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKCK |
| | | VSNKALAAPIEKTISKAKGQPREPQVYTLPPSREEMTK |
| | | NQVSLTCLVKGFYPSDIAVEWESNGQPENNYKTTPPV |
| | | LDSDGSFFLYSKLTVDKSRWQQGNVFSCSVMHEALH |
| | | NHYTQKSLSLSPG |
| SEQ ID | CEAxCD3 kappa | EIVLTQSPATLSLSPGERATLSCRASQSVNSNLNWYQQ |
| NO: 4 | light chain | KPGQAPRLLIYHSNNRPHGIPARFSGSGSGTDFTLTISS |
| | | LEPEDFAVYYCQQFDYFREYNTFGQGTKVEIKRTVAA |
| | | PSVFIFPPSDEQLKSGTASVVCLLNNFYPREAKVQWK |
| | | VDNALQSGNSQESVTEQDSKDSTYSLSSTLTLSKADY |
| | | EKHKVYACEVTHQGLSSPVTKSFNRGEC |
| SEQ ID | CEAxCD3 lambda | QTVVTQEPSLTVSPGGTVTLTCRSSTGAVTTSNYANW |
| NO: 5 | light chain | FQQKPGQAPRGLIGGTNKRAPGTPARFSGSLLGGKAA |
| | | LTLSGAQPEDEAEYYCALWYKQRWVFGGGTKLTVL |
| | | GQPKAAPSVTLFPPSSEELQANKATLVCLISDFYPGAV |
| | | TVAWKADSSPVKAGVETTTPSKQSNNKYAASSYLSLT |
| | | PEQWKSHRSYSCQVTHEGSTVEKTVAPTECS |
| SEQ ID | Common heavy chain | GFTFSSYA |
| NO: 6 | CDR-H1 | |
| SEQ ID | Common heavy chain | ISGSGGST |
| NO: 7 | CDR-H2 | |
| SEQ ID | Common heavy chain | AKSYGAFDY |
| NO: 8 | CDR-H3 | |
| SEQ ID | Common heavy chain | EVQLLESGGGLVQPGGSLRLSCAASGFTFSSYAMSWV |
| NO: 9 | VH | RQAPGKGLEWVSAISGSGGSTYYADSVKGRFTISRDN |
| | | SKNTLYLQMNSLRAEDTAVYYCAKSYGAFDYWGQG |
| | | TLVTVSS |
| SEQ ID | Common heavy chain | EVQLLESGGGLVQPGGSLRLSCAASGFTFSSYAMSWV |
| NO: 10 | HC (wild-type) | RQAPGKGLEWVSAISGSGGSTYYADSVKGRFTISRDN |
| | | SKNTLYLQMNSLRAEDTAVYYCAKSYGAFDYWGQG |
| | | TLVTVSSASTKGPSVFPLAPSSKSTSGGTAALGCLVKD |
| | | YFPEPVTVSWNSGALTSGVHTFPAVLQSSGLYSLSSV |
| | | VTVPSSSLGTQTYICNVNHKPSNTKVDKRVEPKSCDK |
| | | THTCPPCPAPELLGGPSVFLFPPKPKDTLMISRTPEVTC |
| | | VVVDVSHEDPEVKFNWYVDGVEVHNAKTKPREEQY |
| | | NSTYRVVSVLTVLHQDWLNGKEYKCKVSNKALPAPI |
| | | EKTISKAKGQPREPQVYTLPPSREEMTKNQVSLTCLV |
| | | KGFYPSDIAVEWESNGQPENNYKTTPPVLDSDGSFFL |
| | | YSKLTVDKSRWQQGNVFSCSVMHEALHNHYTQKSLS |
| | | LSPG |
| SEQ ID | Common heavy chain | EVQLLESGGGLVQPGGSLRLSCAASGFTFSSYAMSWV |
| NO: 11 | HC (LALA mutation) | RQAPGKGLEWVSAISGSGGSTYYADSVKGRFTISRDN |
| | | SKNTLYLQMNSLRAEDTAVYYCAKSYGAFDYWGQG |
| | | TLVTVSSASTKGPSVFPLAPSSKSTSGGTAALGCLVKD |
| | | YFPEPVTVSWNSGALTSGVHTFPAVLQSSGLYSLSSV |
| | | VTVPSSSLGTQTYICNVNHKPSNTKVDKRVEPKSCDK |
| | | THTCPPCPAPEAAGGPSVFLFPPKPKDTLMISRTPEVTC |
| | | VVVDVSHEDPEVKFNWYVDGVEVHNAKTKPREEQY |
| | | NSTYRVVSVLTVLHQDWLNGKEYKCKVSNKALPAPI |
| | | EKTISKAKGQPREPQVYTLPPSREEMTKNQVSLTCLV |
| | | KGFYPSDIAVEWESNGQPENNYKTTPPVLDSDGSFFL |
| | | YSKLTVDKSRWQQGNVFSCSVMHEALHNHYTQKSLS |
| | | LSPG |
| SEQ ID | Common heavy chain | EVQLLESGGGLVQPGGSLRLSCAASGFTFSSYAMSWV |
| NO: 12 | HC (LALAPA | RQAPGKGLEWVSAISGSGGSTYYADSVKGRFTISRDN |
| | mutation) | SKNTLYLQMNSLRAEDTAVYYCAKSYGAFDYWGQG |
| | | TLVTVSSASTKGPSVFPLAPSSKSTSGGTAALGCLVKD |
| | | YFPEPVTVSWNSGALTSGVHTFPAVLQSSGLYSLSSV |
| | | VTVPSSSLGTQTYICNVNHKPSNTKVDKRVEPKSCDK |
| | | THTCPPCPAPEAAGGPSVFLFPPKPKDTLMISRTPEVTC |
| | | VVVDVSHEDPEVKFNWYVDGVEVHNAKTKPREEQY |
| | | NSTYRVVSVLTVLHQDWLNGKEYKCKVSNKALAAPI |
| | | EKTISKAKGQPREPQVYTLPPSREEMTKNQVSLTCLV |
| | | KGFYPSDIAVEWESNGQPENNYKTTPPVLDSDGSFFL |
| | | YSKLTVDKSRWQQGNVFSCSVMHEALHNHYTQKSLS |
| | | LSPG |
| SEQ ID | PD-L1 binding part | SSDVFRPPL |
| NO: 13 | S79 CDR-L1 | |
| SEQ ID | PD-L1 binding part | FAS |
| NO: 14 | S79 CDR-L2 | |
| SEQ ID | PD-L1 binding part | SSWDHTGDGHV |
| NO: 15 | S79 CDR-L3 | |
| SEQ ID | PD-L1 binding part | QSALTQPASVSGSPGQSITISCTGTSSDVFRPPLVSWYQ |
| NO: 16 | S79 VL (light chain | QHPGKAPKLMIYFASLRPSGVSNRFSGSKSGNTASLTI |
| | variable region) | SGLQAEDEADYYCSSWDHTGDGHVEGGGTKLTVL |
| SEQ ID | PD-L1 binding part | QSALTQPASVSGSPGQSITISCTGTSSDVFRPPLVSWYQ |
| NO: 17 | S79 LC (light chain) | QHPGKAPKLMIYFASLRPSGVSNRFSGSKSGNTASLTI |
| | | SGLQAEDEADYYCSSWDHTGDGHVFGGGTKLTVLG |
| | | QPKAAPSVTLFPPSSEELQANKATLVCLISDFYPGAVT |
| | | VAWKADSSPVKAGVETTTPSKQSNNKYAASSYLSLTP |
| | | EQWKSHRSYSCQVTHEGSTVEKTVAPTECS |
| SEQ ID | CD28 binding part | QSVLYSSNNKNY |
| NO: 18 | AI3 CDR-L1 | |
| SEQ ID | CD28 binding part | WAS |
| NO: 19 | AI3 CDR-L2 | |
| SEQ ID | CD28 binding part | QQNLRPPET |
| NO: 20 | AI3 CDR-L3 | |
| SEQ ID | CD28 binding part | DIVMTQSPDSLAVSLGERATINCKSSQSVLYSSNNKNY |
| NO: 21 | AI3 VL (light chain | LAWYQQKPGQPPKLLIYWASTRESGVPDRFSGSGSGT |
| | variable region) | DFTLTISSLQAEDVAVYYCQQNLRPPETFGQGTKVEIK |
| SEQ ID | CD28 binding part | DIVMTQSPDSLAVSLGERATINCKSSQSVLYSSNNKNY |
| NO: 22 | AI3 LC (light chain) | LAWYQQKPGQPPKLLIYWASTRESGVPDRFSGSGSGT |
| | | DFTLTISSLQAEDVAVYYCQQNLRPPETFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLS |
| | | KADYEKHKVYACEVTHQGLSSPVTKSFNRGEC |
| SEQ ID | CD28 binding part | QSVLYSSNNKNY |
| NO: 23 | AI13 CDR-L1 | |
| SEQ ID | CD28 binding part | WAS |
| NO: 24 | AI13 CDR-L2 | |
| SEQ ID | CD28 binding part | QQNFRPPET |
| NO: 25 | AI13 CDR-L3 | |
| SEQ ID | CD28 binding part | DIVMTQSPDSLAVSLGERATINCKSSQSVLYSSNNKNY |
| NO: 26 | AI13 VL (light chain | LAWYQQKPGQPPKLLIYWASTRESGVPDRFSGSGSGT |
| | variable region) | DFTLTISSLQAEDVGVYYCQQNFRPPETFGGGTKVEIK |
| SEQ ID | CD28 binding part | DIVMTQSPDSLAVSLGERATINCKSSQSVLYSSNNKNY |
| NO: 27 | AI13 LC (light chain) | LAWYQQKPGQPPKLLIYWASTRESGVPDRFSGSGSGT |
| | | DFTLTISSLQAEDVGVYYCQQNFRPPETFGGGTKVEIK |
| | | RTVAAPSVFIFPPSDEQLKSGTASVVCLLNNFYPREAK |
| | | VQWKVDNALQSGNSQESVTEQDSKDSTYSLSSTLTLS |
| | | KADYEKHKVYACEVTHQGLSSPVTKSFNRGEC |
| SEQ ID | CD28 binding part | GDLLTFEGKTY |
| NO: 28 | AI5 CDR-L1 | |
| SEQ ID | CD28 binding part | EVS |
| NO: 29 | AI5 CDR-L2 | |
| SEQ ID | CD28 binding part | MQAHSTRFAFT |
| NO: 30 | AI5 CDR-L3 | |
| SEQ ID | CD28 binding part | SSNIGSNT |
| NO: 31 | AI7 CDR-L1 | |
| SEQ ID | CD28 binding part | SEN |
| NO: 32 | AI7 CDR-L2 | |
| SEQ ID | CD28 binding part | AAWDLNPAIGYV |
| NO: 33 | AI7 CDR-L3 | |
| SEQ ID | CD28 binding part | DDLLRFSGKTY |
| NO: 34 | AI8 CDR-L1 | |
| SEQ ID | CD28 binding part | EVS |
| NO: 35 | AI8 CDR-L2 | |
| SEQ ID | CD28 binding part | MQASRSKVAFT |
| NO: 36 | AI8 CDR-L3 | |
| SEQ ID | CD28 binding part | GDLLSFDGKTY |
| NO: 37 | AI9 CDR-L1 | |
| SEQ ID | CD28 binding part | EVS |
| NO: 38 | AI9 CDR-L2 | |
| SEQ ID | CD28 binding part | MQASKHRFGFT |
| NO: 39 | AI9 CDR-L3 | |
| SEQ ID | CD28 binding part | GDLLEFAGKTY |
| NO: 40 | AI10 CDR-L1 | |
| SEQ ID | CD28 binding part | EVS |
| NO: 41 | AI10 CDR-L2 | |
| SEQ ID | CD28 binding part | MQAHGSKIGFT |
| NO: 42 | AI10 CDR-L3 | |
| SEQ ID | CD28 binding part | QEIHSH |
| NO: 43 | AI11 CDR-L1 | |
| SEQ ID | CD28 binding part | STS |
| NO: 44 | AI11 CDR-L2 | |
| SEQ ID | CD28 binding part | QQGFRFAFST |
| NO: 45 | AI11 CDR-L3 | |
| SEQ ID | CD28 binding part | QSILYSSSNRNY |
| NO: 46 | AI12 CDR-L1 | |
| SEQ ID | CD28 binding part | WAS |
| NO: 47 | AI12 CDR-L2 | |
| SEQ ID | CD28 binding part | QQNFRPPET |
| NO: 48 | AI12 CDR-L3 | |
| SEQ ID | CD28 binding part | QAVFSLD |
| NO: 49 | AI14 CDR-L1 | |
| SEQ ID | CD28 binding part | STS |
| NO: 50 | AI14 CDR-L2 | |
| SEQ ID | CD28 binding part | QQGPAFKPDVT |
| NO: 51 | AI14 CDR-L3 | |
| SEQ ID | CD28 binding part | QSVSSY |
| NO: 52 | AI15 CDR-L1 | |
| SEQ ID | CD28 binding part | DAS |
| NO: 53 | AI15 CDR-L2 | |
| SEQ ID | CD28 binding part | HQRYRPPET |
| NO: 54 | AI15 CDR-L3 | |
| SEQ ID | CD28 binding part | QSIGAY |
| NO: 55 | AI16 CDR-L1 | |
| SEQ ID | CD28 binding part | HAS |
| NO: 56 | AI16 CDR-L2 | |
| SEQ ID | CD28 binding part | QQYKDRPLT |
| NO: 57 | AI16 CDR-L3 | |
| SEQ ID | CD28 binding part | GNLLTHDGKTY |
| NO: 58 | AI17 CDR-L1 | |
| SEQ ID | CD28 binding part | EVS |
| NO: 59 | AI17 CDR-L2 | |
| SEQ ID | CD28 binding part | MQANALKFGFT |
| NO: 60 | AI17 CDR-L3 | |
| SEQ ID | CD28 binding part | SDLLRFEGKTY |
| NO: 61 | AI18 CDR-L1 | |
| SEQ ID | CD28 binding part | EVS |
| NO: 62 | AI18 CDR-L2 | |
| SEQ ID | CD28 binding part | MQAHASALKFT |
| NO: 63 | AI18 CDR-L3 | |
| SEQ ID | CD28 binding part | GNLLTHGGKTY |
| NO: 64 | AI19 CDR-L1 | |
| SEQ ID | CD28 binding part | EVS |
| NO: 65 | AI19 CDR-L2 | |
| SEQ ID | CD28 binding part | MQAHADGFGFT |
| NO: 66 | AI19 CDR-L3 | |
| SEQ ID | CD28 binding part | GDLLRFDGKTY |
| NO: 67 | AI20 CDR-L1 | |
| SEQ ID | CD28 binding part | EVS |
| NO: 68 | AI20 CDR-L2 | |
| SEQ ID | CD28 binding part | MQASETAVRFT |
| NO: 69 | AI20 CDR-L3 | |
| SEQ ID | CD28 binding part | TDLLTFEGKTY |
| NO: 70 | AI21 CDR-L1 | |
| SEQ ID | CD28 binding part | EVS |
| NO: 71 | AI21 CDR-L2 | |
| SEQ ID | CD28 binding part | MQAHSSKFKFT |
| NO: 72 | AI21 CDR-L3 | |
| SEQ ID | CD28 binding part | GDLLSTDGKTY |
| NO: 73 | AI22 CDR-L1 | |
| SEQ ID | CD28 binding part | EVS |
| NO: 74 | AI22 CDR-L2 | |
| SEQ ID | CD28 binding part | MQASGSRFYFT |
| NO: 75 | AI22 CDR-L3 | |
| SEQ ID | CD28 binding part | QTLLYKYDNKNY |
| NO: 76 | AI23 CDR-L1 | |
| SEQ ID | CD28 binding part | WAS |
| NO: 77 | AI23 CDR-L2 | |
| SEQ ID | CD28 binding part | HQYLYPAET |
| NO: 78 | AI23 CDR-L3 | |
| SEQ ID | PD-L1 binding part | SSNIRDSF |
| NO: 79 | S8 CDR-L1 | |
| SEQ ID | PD-L1 binding part | ATN |
| NO: 80 | S8 CDR-L2 | |
| SEQ ID | PD-L1 binding part | AAWHPYYTL |
| NO: 81 | S8 CDR-L3 | |
| SEQ ID | PD-L1 binding part | SSNIRDSF |
| NO: 82 | S9 CDR-L1 | |
| SEQ ID | PD-L1 binding part | ATN |
| NO: 83 | S9 CDR-L2 | |
| SEQ ID | PD-L1 binding part | ASWWPYGTV |
| NO: 84 | S9 CDR-L3 | |
| SEQ ID | PD-L1 binding part | SSNIRDSF |
| NO: 85 | S37 CDR-L1 | |
| SEQ ID | PD-L1 binding part | ATN |
| NO: 86 | S37 CDR-L2 | |
| SEQ ID | PD-L1 binding part | ASWWPFGTV |
| NO: 87 | S37 CDR-L3 | |
| SEQ ID | PD-L1 binding part | SSDVVKNNF |
| NO: 88 | S14 CDR-L1 | |
| SEQ ID | PD-L1 binding part | FGS |
| NO: 89 | S14 CDR-L2 | |
| SEQ ID | PD-L1 binding part | SSWDMPALF |
| NO: 90 | S14 CDR-L3 | |
| SEQ ID | PD-L1 binding part | SSDVVKNNF |
| NO: 91 | S15 CDR-L1 | |
| SEQ ID | PD-L1 binding part | FGS |
| NO: 92 | S15 CDR-L2 | |
| SEQ ID | PD-L1 binding part | SSWDEPDRP |
| NO: 93 | S15 CDR-L3 | |
| SEQ ID | PD-L1 binding part | SSDVVKNNF |
| NO: 94 | S17 CDR-L1 | |
| SEQ ID | PD-L1 binding part | FGS |
| NO: 95 | S17 CDR-L2 | |
| SEQ ID | PD-L1 binding part | SSWDLPFLM |
| NO: 96 | S17 CDR-L3 | |
| SEQ ID | PD-L1 binding part | SSDVVKNNF |
| NO: 97 | S57 CDR-L1 | |
| SEQ ID | PD-L1 binding part | FGS |
| NO: 98 | S57 CDR-L2 | |
| SEQ ID | PD-L1 binding part | SSWKEPDRP |
| NO: 99 | S57 CDR-L3 | |
| SEQ ID | PD-L1 binding part | SSDVVKNNF |
| NO: 100 | S58 CDR-L1 | |
| SEQ ID | PD-L1 binding part | FGS |
| NO: 101 | S58 CDR-L2 | |
| SEQ ID | PD-L1 binding part | SSWDEPDRP |
| NO: 102 | S58 CDR-L3 | |
| SEQ ID | PD-L1 binding part | SSNIAHKP |
| NO: 103 | S28 CDR-L1 | |
| SEQ ID | PD-L1 binding part | HDN |
| NO: 104 | S28 CDR-L2 | |
| SEQ ID | PD-L1 binding part | AAWDFATWPATEV |
| NO: 105 | S28 CDR-L3 | |
| SEQ ID | PD-L1 binding part | SSNIAHKP |
| NO: 106 | S30 CDR-L1 | |
| SEQ ID | PD-L1 binding part | HDN |
| NO: 107 | S30 CDR-L2 | |
| SEQ ID | PD-L1 binding part | AAWDFSRWPATEV |
| NO: 108 | S30 CDR-L3 | |
| SEQ ID | PD-L1 binding part | SVDIAHKP |
| NO: 109 | S94 CDR-L1 | |
| SEQ ID | PD-L1 binding part | HDT |
| NO: 110 | S94 CDR-L2 | |
| SEQ ID | PD-L1 binding part | AAWDFATWPATEV |
| NO: 111 | S94 CDR-L3 | |
| SEQ ID | PD-L1 binding part | SSDVAKIPL |
| NO: 112 | S23 CDR-L1 | |
| SEQ ID | PD-L1 binding part | FAS |
| NO: 113 | S23 CDR-L2 | |
| SEQ ID | PD-L1 binding part | SSWDNAGDGHV |
| NO: 114 | S23 CDR-L3 | |
| SEQ ID | PD-L1 binding part | SSDVLRPPL |
| NO: 115 | S46 CDR-L1 | |
| SEQ ID | PD-L1 binding part | FAS |
| NO: 116 | S46 CDR-L2 | |
| SEQ ID | PD-L1 binding part | SSWDNAGDGHV |
| NO: 117 | S46 CDR-L3 | |
| SEQ ID | PD-L1 binding part | SSDVFRPPL |
| NO: 118 | S71 CDR-L1 | |
| SEQ ID | PD-L1 binding part | FAS |
| NO: 119 | S71 CDR-L2 | |
| SEQ ID | PD-L1 binding part | SSWDQSGDGHV |
| NO: 120 | S71 CDR-L3 | |
| SEQ ID | PD-L1 binding part | QSALTQPASVSGSPGQSITISCTGTSSDVFRPPLVSWYQ |
| NO: 121 | S8 VL (light chain | QHPGKAPKLMIYFASLRPSGVSNRFSGSKSGNTASLTI |
| | variable region) | SGLQAEDEADYYCSSWDHTGDGHVFGGGTKLTVLG |
| | | QPKAAPSVTL |
| SEQ ID | PD-L1 binding part | QSVLTQPPSASGTPGQRVTISCSGSSSNIRDSFVNWYQ |
| NO: 122 | S9 VL (light chain | QLPGTAPKLLIYATNIRPSGVPDRFSGSKSGTSASLAIS |
| | variable region) | GLQSEDEADYYCASWWPYGTVFGGGTKLTVLGQPK |
| | | AAPSVTL |
| SEQ ID | PD-L1 binding part | QSVLTQPPSASGTPGQRVTISCSGSSSNIRDSFVNWYQ |
| NO: 123 | S37 VL (light chain | QLPGTAPKLLIYATNIRPSGVPDRFSGSKSGTSASLAIS |
| | variable region) | GLQSEDEADYYCASWWPFGTVFGGGTKLTVLGQPKA |
| | | APSVTL |
| SEQ ID | PD-L1 binding part | QSALTQPASVSGSPGQSITISCTGTSSDVVKNNFVSWY |
| NO: 124 | S14 VL (light chain | QQHPGKAPKLMIYFGSVRPSGVSNRFSGKSGNTASLTI |
| | variable region) | SGLQAEDEADYYCSSWDMPALFFGGGTKLTVLGQPK |
| | | AAPSVTL |
| SEQ ID | PD-L1 binding part | QSALTQPASVSGSPGQSITISCTGTSSDVVKNNFVSWY |
| NO: 125 | S15 VL (light chain | QQHPGKAPKLMIYFGSVRPSGVSNRFSGSKSGNTASL |
| | variable region) | TISGLQAEDEADYYCSSWDEPDRPFGGGTKLTVLGQP |
| | | KAAPSVTL |
| SEQ ID | PD-L1 binding part | QSALTQPASVSGSPGQSITISCTGTSSDVVKNNFVSWY |
| NO: 126 | S17 VL (light chain | QQHPGKAPKPMIYFGSVRPSGVSNRFSGSKSGNTASL |
| | variable region) | TISGLQAEDEADYYCSSWDLPFLMFGXGTKLTVLGQP |
| | | KAAPSVTL |
| SEQ ID | PD-L1 binding part | QSALTQPASVSGSPGQSITISCTSISSDVVKNNFVSWYQ |
| NO: 127 | S57 VL (light chain | QHPGKAPKLMIYFGSVTADGVSNRFSGSKSGNTASLTI |
| | variable region) | SGLQAEDEADYYCSSWDEPDRPFGGGTKLTVLGQPK |
| | | AAPSVTL |
| SEQ ID | PD-L1 binding part | QSALTQPASVSGSPGQSITISCNSPSSDVVKNNFVSWY |
| NO: 128 | S58 VL (light chain | QQHPGKAPKLMIYFGSVTGPGVSNRFSGSKSGNTASL |
| | variable region) | TISGLQAEDEADYYCSSWDEPDRPFGGGTKLTVLGQP |
| | | KAAPSVTL |
| SEQ ID | PD-L1 binding part | QSVLTQPPSASGTPGQRVTISCSGSSSNIAHKPVNWYQ |
| NO: 129 | S28 VL (light chain | QLPGTAPKLLIYHDNSRPSGVPDRFSGSKSGTSASLAIS |
| | variable region) | GLQSEDEADYYCAAWDFATWPATEVFGGGTKLTVL |
| | | GQPKAAPSVTL |
| SEQ ID | PD-L1 binding part | QSVLTQPPSASGTPGQRVTISCSGSSSNIAHKPVNWYQ |
| NO: 130 | S30 VL (light chain | QLPGTAPKLLIYHDNSRPSGVPDRFSGSRSGTSASLAIS |
| | variable region) | GLQSEDEADYYCAAWDFSRWPATEVFGGGTKLTVLG |
| | | QPKAAPSVTL |
| SEQ ID | PD-L1 binding part | QSVLTQPPSASGTPGQRVTISCISGSVDIAHKPVNWYQ |
| NO: 131 | S94 VL (light chain | QLPGTAPKLLIYHDTSTPDGVPDRFSGSKSGTSASLAIS |
| | variable region) | GLQSEDEADYYCAAWDFATWPATEVFGGGTKLTVL |
| | | GQPKAAPSVTL |
| SEQ ID | PD-L1 binding part | QSALTQPASVSGSPGQSITISCTGTSSDVAKIPLVSWYQ |
| NO: 132 | S23 VL (light chain | QHPGKAPKLMIYFASLRPSGVSNRFSGSKSGNTASLTI |
| | variable region) | SGLQAEDEADYYCSSWDNAGDGHVFGGGTKLTVLG |
| | | QPKAAPSVTL |
| SEQ ID | PD-L1 binding part | QSALTQPASVSGSPGQSITISCTGTSSDVLRPPLVSWYQ |
| NO: 133 | S46 VL (light chain | QHPGKAPKLMIYFASLRPSGVSNRFSGSKSGNTASLTI |
| | variable region) | SGLQAEDEADYYCSSWDNAGDGHVFGGGTKLTVLG |
| | | QPKAAPSVTL |
| SEQ ID | PD-L1 binding part | QSALTQPASVSGSPGQSITISCTGTSSDVFRPPLVSWYQ |
| NO: 134 | S71 VL (light chain | QHPGKAPKLMIYFASLRPSGVSNRFSGSKSGNTASLTI |
| | variable region) | SGLQAEDEADYYCSSWDQSGDGHVFGGGTKLTVLGQ |
| | | PKAAPSVTL |
| SEQ ID | PD-L1 binding part | QSVLTQPPSASGTPGQRVTISCSGSSSNIRDSFVNWYQ |
| NO: 135 | S8 LC (light chain) | QLPGTAPKLLIYATNIRPSGVPDRFSGSKSGTSASLAIS |
| | | GLQSEDEADYYCAAWHPYYTLFGGGTKLTVLGQPKA |
| | | APSVTLFPPSSEELQANKATLVCLISDFYPGAVTVAWK |
| | | ADSSPVKAGVETTTPSKQSNNKYAASSYLSLTPEQWK |
| | | SHRSYSCQVTHEGSTVEKTVAPTECS |
| SEQ ID | PD-L1 binding part | QSVLTQPPSASGTPGQRVTISCSGSSSNIRDSFVNWYQ |
| NO: 136 | S9 LC (light chain) | QLPGTAPKLLIYATNIRPSGVPDRFSGSKSGTSASLAIS |
| | | GLQSEDEADYYCASWWPYGTVFGGGTKLTVLGQPK |
| | | AAPSVTLFPPSSEELQANKATLVCLISDFYPGAVTVAW |
| | | KADSSPVKAGVETTTPSKQSNNKYAASSYLSLTPEQW |
| | | KSHRSYSCQVTHEGSTVEKTVAPTECS |
| SEQ ID | PD-L1 binding part | QSVLTQPPSASGTPGQRVTISCSGSSSNIRDSFVNWYQ |
| NO: 137 | S37 LC (light chain) | QLPGTAPKLLIYATNIRPSGVPDRFSGSKSGTSASLAIS |
| | | GLQSEDEADYYCASWWPFGTVFGGGTKLTVLGQPKA |
| | | APSVTLFPPSSEELQANKATLVCLISDFYPGAVTVAWK |
| | | ADSSPVKAGVETTTPSKQSNNKYAASSYLSLTPEQWK |
| | | SHRSYSCQVTHEGSTVEKTVAPTECS |
| SEQ ID | PD-L1 binding part | QSALTQPASVSGSPGQSITISCTGTSSDVVKNNFVSWY |
| NO: 138 | S14 LC (light chain) | QQHPGKAPKLMIYFGSVRPSGVSNRFSGKSGNTASLTI |
| | | SGLQAEDEADYYCSSWDMPALFFGGGTKLTVLGQPK |
| | | AAPSVTLFPPSSEELQANKATLVCLISDFYPGAVTVAW |
| | | KADSSPVKAGVETTTPSKQSNNKYAASSYLSLTPEQW |
| | | KSHRSYSCQVTHEGSTVEKTVAPTECS |
| SEQ ID | PD-L1 binding part | QSALTQPASVSGSPGQSITISCTGTSSDVVKNNFVSWY |
| NO: 139 | S15 LC (light chain) | QQHPGKAPKLMIYFGSVRPSGVSNRFSGSKSGNTASL |
| | | TISGLQAEDEADYYCSSWDEPDRPFGGGTKLTVLGQP |
| | | KAAPSVTLFPPSSEELQANKATLVCLISDFYPGAVTVA |
| | | WKADSSPVKAGVETTTPSKQSNNKYAASSYLSLTPEQ |
| | | WKSHRSYSCQVTHEGSTVEKTVAPTECS |
| SEQ ID | PD-L1 binding part | QSALTQPASVSGSPGQSITISCTGTSSDVVKNNFVSWY |
| NO: 140 | S17 LC (light chain) | QQHPGKAPKPMIYFGSVRPSGVSNRFSGSKSGNTASL |
| | | TISGLQAEDEADYYCSSWDLPFLMFGXGTKLTVLGQP |
| | | KAAPSVTLFPPSSEELQANKATLVCLISDFYPGAVTVA |
| | | WKADSSPVKAGVETTTPSKQSNNKYAASSYLSLTPEQ |
| | | WKSHRSYSCQVTHEGSTVEKTVAPTECS |
| SEQ ID | PD-L1 binding part | QSALTQPASVSGSPGQSITISCTSISSDVVKNNFVSWYQ |
| NO: 141 | S57 LC (light chain) | QHPGKAPKLMIYFGSVTADGVSNRFSGSKSGNTASLTI |
| | | SGLQAEDEADYYCSSWDEPDRPFGGGTKLTVLGQPK |
| | | AAPSVTLFPPSSEELQANKATLVCLISDFYPGAVTVAW |
| | | KADSSPVKAGVETTTPSKQSNNKYAASSYLSLTPEQW |
| | | KSHRSYSCQVTHEGSTVEKTVAPTECS |
| SEQ ID | PD-L1 binding part | QSALTQPASVSGSPGQSITISCNSPSSDVVKNNFVSWY |
| NO: 142 | S58 LC (light chain) | QQHPGKAPKLMIYFGSVTGPGVSNRFSGSKSGNTASL |
| | | TISGLQAEDEADYYCSSWDEPDRPFGGGTKLTVLGQP |
| | | KAAPSVTLFPPSSEELQANKATLVCLISDFYPGAVTVA |
| | | WKADSSPVKAGVETTTPSKQSNNKYAASSYLSLTPEQ |
| | | WKSHRSYSCQVTHEGSTVEKTVAPTECS |
| SEQ ID | PD-L1 binding part | QSVLTQPPSASGTPGQRVTISCSGSSSNIAHKPVNWYQ |
| NO: 143 | S28 LC (light chain) | QLPGTAPKLLIYHDNSRPSGVPDRFSGSKSGTSASLAIS |
| | | GLQSEDEADYYCAAWDFATWPATEVFGGGTKLTVL |
| | | GQPKAAPSVTLFPPSSEELQANKATLVCLISDFYPGAV |
| | | TVAWKADSSPVKAGVETTTPSKQSNNKYAASSYLSLT |
| | | PEQWKSHRSYSCQVTHEGSTVEKTVAPTECS |
| SEQ ID | PD-L1 binding part | QSVLTQPPSASGTPGQRVTISCSGSSSNIAHKPVNWYQ |
| NO: 144 | S30 LC (light chain) | QLPGTAPKLLIYHDNSRPSGVPDRFSGSRSGTSASLAIS |
| | | GLQSEDEADYYCAAWDFSRWPATEVFGGGTKLTVLG |
| | | QPKAAPSVTLFPPSSEELQANKATLVCLISDFYPGAVT |
| | | VAWKADSSPVKAGVETTTPSKQSNNKYAASSYLSLTP |
| | | EQWKSHRSYSCQVTHEGSTVEKTVAPTECS |
| SEQ ID | PD-L1 binding part | QSVLTQPPSASGTPGQRVTISCISGSVDIAHKPVNWYQ |
| NO: 145 | S94 LC (light chain) | QLPGTAPKLLIYHDTSTPDGVPDRFSGSKSGTSASLAIS |
| | | GLQSEDEADYYCAAWDFATWPATEVFGGGTKLTVL |
| | | GQPKAAPSVTLFPPSSEELQANKATLVCLISDFYPGAV |
| | | TVAWKADSSPVKAGVETTTPSKQSNNKYAASSYLSLT |
| | | PEQWKSHRSYSCQVTHEGSTVEKTVAPTECS |
| SEQ ID | PD-L1 binding part | QSALTQPASVSGSPGQSITISCTGTSSDVAKIPLVSWYQ |
| NO: 146 | S23 LC (light chain) | QHPGKAPKLMIYFASLRPSGVSNRFSGSKSGNTASLTI |
| | | SGLQAEDEADYYCSSWDNAGDGHVFGGGTKLTVLG |
| | | QPKAAPSVTLFPPSSEELQANKATLVCLISDFYPGAVT |
| | | VAWKADSSPVKAGVETTTPSKQSNNKYAASSYLSLTP |
| | | EQWKSHRSYSCQVTHEGSTVEKTVAPTEC |
| SEQ ID | PD-L1 binding part | QSALTQPASVSGSPGQSITISCTGTSSDVLRPPLVSWYQ |
| NO: 147 | S46 LC (light chain) | QHPGKAPKLMIYFASLRPSGVSNRFSGSKSGNTASLTI |
| | | SGLQAEDEADYYCSSWDNAGDGHVFGGGTKLTVLG |
| | | QPKAAPSVTLFPPSSEELQANKATLVCLISDFYPGAVT |
| | | VAWKADSSPVKAGVETTTPSKQSNNKYAASSYLSLTP |
| | | EQWKSHRSYSCQVTHEGSTVEKTVAPTECS |
| SEQ ID | PD-L1 binding part | QSALTQPASVSGSPGQSITISCTGTSSDVFRPPLVSWYQ |
| NO: 148 | S71 LC (light chain) | QHPGKAPKLMIYFASLRPSGVSNRFSGSKSGNTASLTI |
| | | SGLQAEDEADYYCSSWDQSGDGHVFGGGTKLTVLGQ |
| | | PKAAPSVTLFPPSSEELQANKATLVCLISDFYPGAVTV |
| | | AWKADSSPVKAGVETTTPSKQSNNKYAASSYLSLTPE |
| | | QWKSHRSYSCQVTHEGSTVEKTVAPTECS |
| SEQ ID | CD28 binding part | DIVMTQTPLSLSVTPGQPASISCKSSGDLLTFEGKTYL |
| NO: 149 | AI5 VL (light chain | YWYLQKPGQPPQLLIYEVSNRFSGVPDRFSGSGSGTD |
| | variable region) | FTLKISRVEAEDVGVYYCMQAHSTRFAFTFGQGTKVE |
| | | IK |
| SEQ ID | CD28 binding part | QSVLTQPPSASGTPGQRVTISCSGSSSNIGSNTVNWYQ |
| NO: 150 | AI7 VL (light chain | QLPGTAPKLLIYSENTRPSGVPDRFSGSKSGTSASLAIS |
| | variable region) | GLQSEDEADYYCAAWDLNPAIGYVFGGGTKLTVL |
| SEQ ID | CD28 binding part | DIVMTQTPLSLSVTPGQPASISCKSSDDLLRFSGKTYL |
| NO: 151 | AI8 VL (light chain | YWYLQKPGQPPQLLIYEVSNRFSGVPDRFSGSGSGTD |
| | variable region) | FTLKISRVEAEDVGVYYCMQASRSKVAFTFGQGTKVE |
| | | IK |
| SEQ ID | CD28 binding part | DIVMTQTPLSLSVTPGQPASISCKSSGDLLSFDGKTYL |
| NO: 152 | AI9 VL (light chain | YWYLQKPGQPPQLLIYEVSNRFSGVPDRFSGSGSGTD |
| | variable region) | FTLKISRVEAEDVGVYYCMQASKHRFGFTFGQGTKVE |
| | | IK |
| SEQ ID | CD28 binding part | DIVMTQTPLSLSVTPGQPASISCKSSGDLLEFAGKTYL |
| NO: 153 | AI10 VL (light chain | YWYLQKPGQPPQLLIYEVSNRFSGVPDRFSGSGSGTD |
| | variable region) | FTLKISRVEAEDVGVYYCMQAHGSKIGFTFGQGTKVE |
| | | IK |
| SEQ ID | CD28 binding part | DIQMTQSPSTLSASVGDRVTITCRASQEIHSHLSWYQQ |
| NO: 154 | AI11 VL (light chain | KPGKAPKLLIYSTSTLESGVPSRFSGSGSGTEFTLTISSL |
| | variable region) | QPDDFATYYCQQGFRFAFSTFGQGTKVEIK |
| SEQ ID | CD28 binding part | DIVMTQSPDSLGVSLGERATINCKSSQSILYSSSNRNYL |
| NO: 155 | AI12 VL (light chain | AWYQQKPGQPPKLLIYWASTRESGVPDRFSGSGSGTD |
| | variable region) | FTLTISSLQAEDVGVYYCQQNFRPPETFGGGTKVEIK |
| SEQ ID | CD28 binding part | EIVLTQSPGTLSLSPGERATLSCRASQAVFSLDLHWYQ |
| NO: 156 | AI14 VL (light chain | QKPGQAPRLLIYSTSKRATGIPDRFSGSGSGTDFTLTIS |
| | variable region) | RLEPEDFAVYYCQQGPAFKPDVTFGQGTKVEIK |
| SEQ ID | CD28 binding part | EIVLTQSPATLSLSPGERATLSCRASQSVSSYLAWYQQ |
| NO: 157 | AI15 VL (light chain | KPGQAPRLLIYDASNRATGIPARFSGSGSGTDFTLTISS |
| | variable region) | LEPEDFAVYYCHQRYRPPETFGQGTKVEIK |
| SEQ ID | CD28 binding part | DIQMTQSPSSLSASVGDRVTITCRASQSIGAYLNWYQ |
| NO: 158 | AI16 VL (light chain | QKPGKAPKLLIYHASDLQSGVPSRFSGSGSGTDFTLTIS |
| | variable region) | SLQPEDFATYYCQQYKDRPLTFGQGTKVEIK |
| SEQ ID | CD28 binding part | DIVMTQTPLSLSVTPGQPASISCKSSGNLLTHDGKTYL |
| NO: 159 | AI17 VL (light chain | YWYLQKPGQPPQLLIYEVSNRFSGVPDRFSGSGSGTD |
| | variable region) | FTLKISRVEAEDVGVYYCMQANALKFGFTFGQGTKV |
| | | EIK |
| SEQ ID | CD28 binding part | DIVMTQTPLSLSVTPGQPASISCKSSSDLLRFEGKTYLY |
| NO: 160 | AI18 VL (light chain | WYLQKPGQPPQLLIYEVSNRFSGVPDRFSGSGSGTDFT |
| | variable region) | LKISRVEAEDVGVYYCMQAHASALKFTFGQGTKVEIK |
| SEQ ID | CD28 binding part | DIVMTQTPLSLSVTPGQPASISCKSSGNLLTHGGKTYL |
| NO: 161 | AI19 VL (light chain | YWYLQKPGQPPQLLIYEVSNRFSGVPDRFSGSGSGTD |
| | variable region) | FTLKISRVEAEDVGVYYCMQAHADGFGFTFGQGTKV |
| | | EIK |
| SEQ ID | CD28 binding part | DIVMTQTPLSLSVTPGQPASISCKSSGDLLRFDGKTYL |
| NO: 162 | AI20 VL (light chain | YWYLQKPGQPPQLLIYEVSNRFSGVPDRFSGSGSGTD |
| | variable region) | FTLKISRVEAEDVGVYYCMQASETAVRFTFGQGTKVE |
| | | IK |
| SEQ ID | CD28 binding part | DIVMTQTPLSLSVTPGQPASISCKSSTDLLTFEGKTYLY |
| NO: 163 | AI21 VL (light chain | WYLQKPGQPPQLLIYEVSNRFSGVPDRFSGSGSGTDFT |
| | variable region) | LKISRVEAEDVGVYYCMQAHSSKFKFTFGQGTKVEIK |
| SEQ ID | CD28 binding part | DIVMTQTPLSLSVTPGQPASISCKSSGDLLSTDGKTYL |
| NO: 164 | AI22 VL (light chain | YWYLQKPGQPPQLLIYEVSNRFSGVPDRFSGSGSGTD |
| | variable region) | FTLKISRVEAEDVGVYYCMQASGSRFYFTFGQGTKVE |
| | | IK |
| SEQ ID | CD28 binding part | DIVMTQSPDSLAVSLGERAIINCKSSQTLLYKYDNKNY |
| NO: 165 | AI23 VL (light chain | LAWYQHKPGQPPKLLFYWASTRESGVPDRFSGSGSGT |
| | variable region) | DFTLTINSLQAEDVAVYYCHQYLYPAETFGQGTKLEI |
| | | K |
| SEQ ID | CD28 binding part | DIVMTQTPLSLSVTPGQPASISCKSSGDLLTFEGKTYL |
| NO: 166 | AI5 LC (light chain) | YWYLQKPGQPPQLLIYEVSNRFSGVPDRFSGSGSGTD |
| | | FTLKISRVEAEDVGVYYCMQAHSTRFAFTFGQGTKVE |
| | | IKRTVAAPSVFIFPPSDEQLKSGTASVVCLLNNFYPRE |
| | | AKVQWKVDNALQSGNSQESVTEQDSKDSTYSLSSTL |
| | | TLSKADYEKHKVYACEVTHQGLSSPVTKSFNRGEC |
| SEQ ID | CD28 binding part | QSVLTQPPSASGTPGQRVTISCSGSSSNIGSNTVNWYQ |
| NO: 167 | AI7 LC (light chain) | QLPGTAPKLLIYSENTRPSGVPDRFSGSKSGTSASLAIS |
| | | GLQSEDEADYYCAAWDLNPAIGYVFGGGTKLTVLGQ |
| | | PKAAPSVTLFPPSSEELQANKATLVCLISDFYPGAVTV |
| | | AWKADSSPVKAGVETTTPSKQSNNKYAASSYLSLTPE |
| | | QWKSHRSYSCQVTHEGSTVEKTVAPTECS |
| SEQ ID | CD28 binding part | DIVMTQTPLSLSVTPGQPASISCKSSDDLLRFSGKTYL |
| NO: 168 | AI8 LC (light chain) | YWYLQKPGQPPQLLIYEVSNRFSGVPDRFSGSGSGTD |
| | | FTLKISRVEAEDVGVYYCMQASRSKVAFTFGQGTKVE |
| | | IKRTVAAPSVFIFPPSDEQLKSGTASVVCLLNNFYPRE |
| | | AKVQWKVDNALQSGNSQESVTEQDSKDSTYSLSSTL |
| | | TLSKADYEKHKVYACEVTHQGLSSPVTKSENRGEC |
| SEQ ID | CD28 binding part | DIVMTQTPLSLSVTPGQPASISCKSSGDLLSFDGKTYL |
| NO: 169 | AI9 LC (light chain) | YWYLQKPGQPPQLLIYEVSNRFSGVPDRFSGSGSGTD |
| | | FTLKISRVEAEDVGVYYCMQASKHRFGFTFGQGTKVE |
| | | IKRTVAAPSVFIFPPSDEQLKSGTASVVCLLNNFYPRE |
| | | AKVQWKVDNALQSGNSQESVTEQDSKDSTYSLSSTL |
| | | TLSKADYEKHKVYACEVTHQGLSSPVTKSFNRGEC |
| SEQ ID | CD28 binding part | DIVMTQTPLSLSVTPGQPASISCKSSGDLLEFAGKTYL |
| NO: 170 | AI10 LC (light chain) | YWYLQKPGQPPQLLIYEVSNRFSGVPDRFSGSGSGTD |
| | | FTLKISRVEAEDVGVYYCMQAHGSKIGFTFGQGTKVE |
| | | IKRTVAAPSVFIFPPSDEQLKSGTASVVCLLNNFYPRE |
| | | AKVQWKVDNALQSGNSQESVTEQDSKDSTYSLSSTL |
| | | TLSKADYEKHKVYACEVTHQGLSSPVTKSFNRGEC |
| SEQ ID | CD28 binding part | DIQMTQSPSTLSASVGDRVTITCRASQEIHSHLSWYQQ |
| NO: 171 | AI11 LC (light chain) | KPGKAPKLLIYSTSTLESGVPSRFSGSGSGTEFTLTISSL |
| | | QPDDFATYYCQQGFRFAFSTFGQGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |
| SEQ ID | CD28 binding part | DIVMTQSPDSLGVSLGERATINCKSSQSILYSSSNRNYL |
| NO: 172 | AI12 LC (light chain) | AWYQQKPGQPPKLLIYWASTRESGVPDRFSGSGSGTD |
| | | FTLTISSLQAEDVGVYYCQQNFRPPETFGGGTKVEIKR |
| | | TVAAPSVFIFPPSDEQLKSGTASVVCLLNNFYPREAKV |
| | | QWKVDNALQSGNSQESVTEQDSKDSTYSLSSTLTLSK |
| | | ADYEKHKVYACEVTHQGLSSPVTKSFNRGEC |
| SEQ ID | CD28 binding part | EIVLTQSPGTLSLSPGERATLSCRASQAVFSLDLHWYQ |
| NO: 173 | AI14 LC (light chain) | QKPGQAPRLLIYSTSKRATGIPDRFSGSGSGTDFTLTIS |
| | | RLEPEDFAVYYCQQGPAFKPDVTFGQGTKVEIKRTVA |
| | | APSVFIFPPSDEQLKSGTASVVCLLNNFYPREAKVQW |
| | | KVDNALQSGNSQESVTEQDSKDSTYSLSSTLTLSKAD |
| | | YEKHKVYACEVTHQGLSSPVTKSFNRGEC |
| SEQ ID | CD28 binding part | EIVLTQSPATLSLSPGERATLSCRASQSVSSYLAWYQQ |
| NO: 174 | AI15 LC (light chain) | KPGQAPRLLIYDASNRATGIPARFSGSGSGTDFTLTISS |
| | | LEPEDFAVYYCHQRYRPPETFGQGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |
| SEQ ID | CD28 binding part | DIQMTQSPSSLSASVGDRVTITCRASQSIGAYLNWYQ |
| NO: 175 | AI16 LC (light chain) | QKPGKAPKLLIYHASDLQSGVPSRFSGSGSGTDFTLTIS |
| | | SLQPEDFATYYCQQYKDRPLTFGQGTKVEIKRTVAAP |
| | | SVFIFPPSDEQLKSGTASVVCLLNNFYPREAKVQWKV |
| | | DNALQSGNSQESVTEQDSKDSTYSLSSTLTLSKADYE |
| | | KHKVYACEVTHQGLSSPVTKSFNRGEC |
| SEQ ID | CD28 binding part | DIVMTQTPLSLSVTPGQPASISCKSSGNLLTHDGKTYL |
| NO: 176 | AI17 LC (light chain) | YWYLQKPGQPPQLLIYEVSNRFSGVPDRFSGSGSGTD |
| | | FTLKISRVEAEDVGVYYCMQANALKFGFTFGQGTKV |
| | | EIKRTVAAPSVFIFPPSDEQLKSGTASVVCLLNNFYPRE |
| | | AKVQWKVDNALQSGNSQESVTEQDSKDSTYSLSSTL |
| | | TLSKADYEKHKVYACEVTHQGLSSPVTKSFNRGEC |
| SEQ ID | CD28 binding part | DIVMTQTPLSLSVTPGQPASISCKSSSDLLRFEGKTYLY |
| NO: 177 | AI18 LC (light chain) | WYLQKPGQPPQLLIYEVSNRFSGVPDRFSGSGSGTDFT |
| | | LKISRVEAEDVGVYYCMQAHASALKFTFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLS |
| | | KADYEKHKVYACEVTHQGLSSPVTKSFNRGEC |
| SEQ ID | CD28 binding part | DIVMTQTPLSLSVTPGQPASISCKSSGNLLTHGGKTYL |
| NO: 178 | AI19 LC (light chain) | YWYLQKPGQPPQLLIYEVSNRFSGVPDRFSGSGSGTD |
| | | FTLKISRVEAEDVGVYYCMQAHADGFGFTFGQGTKV |
| | | EIKRTVAAPSVFIFPPSDEQLKSGTASVVCLLNNFYPRE |
| | | AKVQWKVDNALQSGNSQESVTEQDSKDSTYSLSSTL |
| | | TLSKADYEKHKVYACEVTHQGLSSPVTKSFNRGEC |
| SEQ ID | CD28 binding part | DIVMTQTPLSLSVTPGQPASISCKSSGDLLRFDGKTYL |
| NO: 179 | AI20 LC (light chain) | YWYLQKPGQPPQLLIYEVSNRFSGVPDRFSGSGSGTD |
| | | FTLKISRVEAEDVGVYYCMQASETAVRFTFGQGTKVE |
| | | IKRTVAAPSVFIFPPSDEQLKSGTASVVCLLNNFYPRE |
| | | AKVQWKVDNALQSGNSQESVTEQDSKDSTYSLSSTL |
| | | TLSKADYEKHKVYACEVTHQGLSSPVTKSFNRGEC |
| SEQ ID | CD28 binding part | DIVMTQTPLSLSVTPGQPASISCKSSTDLLTFEGKTYLY |
| NO: 180 | AI21 LC (light chain) | WYLQKPGQPPQLLIYEVSNRFSGVPDRFSGSGSGTDFT |
| | | LKISRVEAEDVGVYYCMQAHSSKFKFTFGQGTKVEIK |
| | | RTVAAPSVFIFPPSDEQLKSGTASVVCLLNNFYPREAK |
| | | VQWKVDNALQSGNSQESVTEQDSKDSTYSLSSTLTLS |
| | | KADYEKHKVYACEVTHQGLSSPVTKSFNRGEC |
| SEQ ID | CD28 binding part | DIVMTQTPLSLSVTPGQPASISCKSSGDLLSTDGKTYL |
| NO: 181 | AI22 LC (light chain) | YWYLQKPGQPPQLLIYEVSNRFSGVPDRFSGSGSGTD |
| | | FTLKISRVEAEDVGVYYCMQASGSRFYFTFGQGTKVE |
| | | IKRTVAAPSVFIFPPSDEQLKSGTASVVCLLNNFYPRE |
| | | AKVQWKVDNALQSGNSQESVTEQDSKDSTYSLSSTL |
| | | TLSKADYEKHKVYACEVTHQGLSSPVTKSFNRGEC |
| SEQ ID | CD28 binding part | DIVMTQSPDSLAVSLGERAIINCKSSQTLLYKYDNKNY |
| NO: 182 | AI23 LC (light chain) | LAWYQHKPGQPPKLLFYWASTRESGVPDRFSGSGSGT |
| | | DFTLTINSLQAEDVAVYYCHQYLYPAETFGQGTKLEI |
| | | KRTVAAPSVFIFPPSDEQLKSGTASVVCLLNNFYPREA |
| | | KVQWKVDNALQSGNSQESVTEQDSKDSTYSLSSTLTL |
| | | SKADYEKHKVYACEVTHQGLSSPVTKSFNRGEC |

### Definitions

As used herein, including the appended claims, the singular forms of words such as “a,” “an,” and “the,” include their corresponding plural references unless the context clearly dictates otherwise.

As used herein, “TGN1412”, refers to a superagonistic (SA) anti-huCD28 antibody in a human IgC4 isotype as described in W2006050949, that comprises the amino acid sequences of SEQ ID NOs.: 1 and 2.

As used herein, “CEAxCD3”, refers to a CEAxCD3 bispecific KA body originally described in WO2021053587 (which is hereby incorporated by reference in its entirety), and comprises a common heavy chain of SEQ ID NO: 3, a kappa light chain of SEQ ID NO: 4, a lambda light chain of SEQ ID NO: 5.

As used herein, “S79”, refers to a κλ body compatible (common dummy heavy chain) anti-human PD-L1 blocking antibody of high affinity that is human/cyno/mouse cross-reactive. It was originally described in WO2022200389 (which is hereby incorporated by reference in its entirety) and comprises as variable heavy (VH) and variable light (VL) the amino acid sequences of SEQ ID NOs: 9 and 16, respectively.

As used herein, “AI3” and “AI13”, refers to 0, body compatible (common dummy heavy chain) anti-human CD28 agonist antibodies that are human/cyno cross-reactive. They were originally described in WO2023170474 (which is hereby incorporated by reference in their entireties). AI3 comprises as variable heavy (VI) and variable light (VL) the amino acid sequences of SEQ ID NOs: 9 and 21, respectively, while AI13 comprises as variable heavy (VH) and variable light (VL) the amino acid sequences of SEQ ID NOs: 9 and 26, respectively.

As used herein, “/N”, refers to a set of mutations (Leu234Ala+Leu235Ala+Pro329Ala) (i.e., LALAPA) introduced in the human IgG1 Fe portion of a given antibody to abrogate Fc-mediated effector function.

As utilized in accordance with the present disclosure, the following terms, unless otherwise indicated, shall be understood to have the following meanings:

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” or “immunospecifically bind” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal, chimeric, dAb (domain antibody), single chain, F_{ab}, F_{ab′} and F_{(ab′)2} fragments, scFvs, and an Fb expression library, Antibodies with high affinity have an affinity of about 0.0 1 nM-25 nM.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen binding region” or “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” Various methods are known in the art for numbering the amino acids sequences of antibodies and identification of the complementary determining regions. For example, the Kabat numbering system (See Kabat, E. A., et al., Sequences of Protein of immunological interest, Fifth Edition, US Department of Health and Human Services, US Government Printing Office (1991)) or the IMGT numbering system (See IMGT®, the international ImMunoGeneTics information system Available online: http://www.imgt.org/). The IMGT numbering system is routinely used and accepted as a reliable and accurate system in the art to determine amino acid positions in coding sequences, alignment of alleles, and to easily compare sequences in immunoglobulin (IG) and T-cell receptor (TR) from all vertebrate species. The accuracy and the consistency of the IMGT data are based on IMGT-ONTOLOGY, the first, and so far unique, ontology for immunogenetics and immunoinformatics (See Lefranc. M. P. et al., Biomolecules, 2014 December; 4(4), 1102-1139). IMGT tools and databases run against IMFT reference directories built from a large repository of sequences. In the IMGT system the IG V-DOMAIN and IG C-DOMAIN are delimited taking into account the exon delimitation, whenever appropriate. Therefore, the availability of more sequences to the IMGT database, the IMGT exon numbering system can be and “is used” by those skilled in the art reliably to determine amino acid positions in coding sequences and for alignment of alleles. Additionally, correspondences between the IMGT unique numbering with other numberings (i.e., Kabat) are available in the IMGT Scientific chart (See Lefranc. M. P. et al., Biomolecules, 2014 Dee; 4(4), 1102-1139).

The term “hypervariable region” or “variable region” refers to the amino acid residues of an antibody that are typically responsible for antigen-binding. The hypervariable region generally comprises amino acid residues from a “complementarity determining region” or “CDR” (e.g., around about residues 24-34 (LI), 50-56 (L2) and 89-97 (L3) in the VL, and around about 31-35 (HI), 50-65 (H2) and 95-102 (H3) in the VH when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g, residues 24-34 (LI), 50-56 (L2) and 89-97 (L3) in the V_{L}, and 26-32 (HI), 52-56 (H22) and 95-101 (H3) in the V_{H} when numbered in accordance with the Chothia numbering system; Chothia and Lesk, J. Mol. Biol. 196:901-917 (1987)); and/or those residues from a “hypervariable loop” VCDR (e.g., residues 27-38 (LI), 56-65 (L2) and 105-120 (L3) in the V_{L}, and 27-38 (HI), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally, the antibody has symmetrical insertions at one or more of the following points 28, 36 (LI), 63, 74-75 (L2) and 123 (L3) in the V_{L}, and 28, 36 (HI), 63, 74-75 (H2) and 123 (H3) in the V_{H} when numbered in accordance with AHo; Honneger, A. and Plunkthun, A. J. Mol. Biol. 309:657-670 (2001)).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide. An antibody, or a single antibody arm, may, depending on the design, specifically bind an antigen when the dissociation constant is ≤1 μM; e.g., ≤100 nM, preferably ≤10 nM and more preferably ≤1 nM. In some embodiments, the antibody, or a single antibody arm, may bind an antigen with a dissociation constant that is greater than 20 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody, or single antibody arm, of the present invention is to specifically bind to its target, when the equilibrium binding constant (K_{d}) is ≤1 μM, e.g., ≤100 nM, preferably ≤10 nM, and more preferably ≤1 nM, as measured by assays such as radioligand binding assays or similar assays known to those skilled in the art. In some embodiments, the antibody, or a single antibody arm, may bind an antigen with a dissociation constant that is greater than 20 nM.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the invention include the nucleic acid molecules encoding the heavy chain immunoglobulin molecules, and nucleic acid molecules encoding the light chain immunoglobulin molecules described herein.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the invention comprise the heavy chain immunoglobulin molecules, and the light chain immunoglobulin molecules described herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means a polymeric boron of nucleotides of at least 10 bases in length, either ribonucleotides or deoxynucleotides or a modified form of either type of nucleotide. The term includes single and double stranded forms of DNA.

As used herein, the twenty conventional amino acids and their abbreviations follow conventional usage. See Immunology—A Synthesis (2nd Edition, E. S. Golub and DR. Gren, Eds., Sinauer Associates, Sunderland Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motif; and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (198-4)); Introduction to Protein Structure (C. Branden and J. Tooze, eds., Garland Publishing, New York, N.Y. (1991)); and Thornton et at. Nature 354:105 (1991).

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed. McGraw-Hill, San Francisco (1985)).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term patient includes human and veterinary subjects.

### Antibodies

Various procedures known within the art may be used for the production of polyclonal or monoclonal antibodies directed against a given target, such as, for example, CD47, a tumor associated antigen or other target, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, NY, incorporated herein by reference).

Antibodies are purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

In some embodiments, the antibodies of the invention are monoclonal antibodies. Monoclonal antibodies are generated, for example, by using the procedures set forth in the Examples provided herein. Antibodies are also generated, e.g., by immunizing BALB/c mice with combinations of cell transfectants expressing high levels of a given target on their surface. Hybridomas resulting from myeloma/B cell fusions are then screened for reactivity to the selected target.

Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Monoclonal antibodies of the invention include humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization is performed, e.g., by following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539). In some instances, Fv framework residues of the human immunoglobulin are replaced by corresponding non-human residues. Humanized antibodies also comprise, e.g., residues which are found neither in the recipient antibody nor in the imported CDR or framework sequences. In general, the humanized antibody includes substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also includes at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Fully human antibodies are antibody molecules in which the entire sequence of both the light chain and the heavy chain, including the CDRs, arise from human genes. Such antibodies are termed “human antibodies”, or “fully human antibodies” herein. Monoclonal antibodies can be prepared by using trioma technique; the human B-cell hybridoma technique (see Kozbor, et al., 1983 Immunol Today 4: 72); and the EBV hybridoma technique to produce monoclonal antibodies (see Cole, et al., 1985 In: MONOCLONAL ANTIBODIES AND CANCER THERAPY, Alan R. Liss, Inc., pp. 77-96). Monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al, 1985 In: MONOCLONAL ANTIBODIES AND CANCER THERAPY, Alan R. Liss, Inc., pp. 77-96).

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994), Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. An example of such a nonhuman animal is a mouse termed the Xenomouse™ as disclosed in PCT publications WO 96/33735 and WO 96/134096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds specifically to the relevant epitope with high affinity are disclosed in PCT publication WO 99/53049.

The antibody can be expressed by a vector containing a DNA segment encoding the single chain antibody described above.

These can include vectors, liposomes, naked DNA, adjuvant-assisted DNA. gene gun, catheters, etc. Vectors include chemical conjugates such as described in WO 93/64701, which has targeting moiety (e.g., a ligand to a cellular surface receptor), and a nucleic acid binding moiety (e.g., polylysine), viral vector (e.g., a DNA or RNA viral vector), fusion proteins such as described in PCT/US 95/02140 (WO 95/22618) which is a fusion protein containing a target moiety (e.g., an antibody specific for a target cell) and a nucleic acid binding moiety (e.g., a protamine), plasmids, phage, etc. The vectors can be chromosomal, non-chromosomal or synthetic.

Preferred vectors include viral vectors, fusion proteins and chemical conjugates. Retroviral vectors include moloney murine leukemia viruses. DNA viral vectors are preferred. These vectors include pox vectors such as orthopox or avipox vectors, herpesvirus vectors such as a herpes simplex I virus (ISV) vector (see Geller, A. 1 et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et el. Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J Virol. 69:2004 (1995) and Adeno-associated Virus Vectors (see Kaplitt, M. G. et al., Nat, Genet. 8:148 (1994).

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors are preferred for introducing the nucleic acid into neural cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g., infection, transfection, transduction or transformation. Examples of modes of gene transfer include e.g., naked DNA, CaPO₄ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g., adenovirus, HSV) to a desired locator. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

Bispecific antibodies are antibodies that have binding specificities for at least two different antigens. In the present case, one of the binding specificities is for a target such as CD28 or any fragment thereof. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et alt, EMBO J., 10:3655-3659 (1991).

Bispecific and/or monospecific antibodies of the invention can be made using any of a variety of art-recognized techniques, including those disclosed in co-pending application WO 2012/023053, filed Aug. 16, 2011, the contents of which are hereby incorporated by reference in their entirety. The methods described in WO 2012/023053 generate bispecific antibodies that are identical in structure to a human immunoglobulin. This type of molecule is composed of two copies of a unique heavy chain polypeptide, a first light chain variable region fused to a constant Kappa domain and second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions. However it is also possible to obtain bispecific antibodies of the invention by fusing a Kappa light chain variable domain to a constant Lambda domain for a first specificity and fusing a Lambda light chain variable domain to a constant Kappa domain for the second specificity. The bispecific antibodies described in WO 2012/023053 are referred to as IgGκλ antibodies or “κλ bodies,” a new fully human bispecific IgG format. This κλ-body format allows the affinity purification of a bispecific antibody that is undistinguishable from a standard IgG molecule with characteristics that are undistinguishable from a standard monoclonal antibody and, therefore, favorable as compared to previous formats.

An essential step of the method is the identification of two antibody Fv regions (each composed by a variable light chain and variable heavy chain domain) having different antigen specificities that share the same heavy chain variable domain. Numerous methods have been described for the generation of monoclonal antibodies and fragments thereof. (See, e.g., Antibodies: A Laboratory Manual, Harlow E, and Lane D, 1988, Cold Spring Harbor Laboratory Press, Cold Spring Harbor, NY, incorporated herein by reference). Fully human antibodies are antibody molecules in which the sequence of both the light chain and the heavy chain, including the CDRs 1 and 2, arise from human genes. The CDR3 region can be of human origin or designed by synthetic means. Such antibodies are termed “human antibodies”, or “fully human antibodies” herein. Human monoclonal antibodies can be prepared by using the trioma technique; the human B-cell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

Monoclonal antibodies are generated, e.g., by immunizing an animal with a target antigen or an immunogenic fragment, derivative or variant thereof. Alternatively, the animal is immunized with cells transfected with a vector containing a nucleic acid molecule encoding the target antigen, such that the target antigen is expressed and associated with the surface of the transfected cells. A variety of techniques are well-known in the art for producing xenogenic non-human animals. For example, see U.S. Pat. Nos. 6,075,181 and 6,150,584, which is hereby incorporated by reference in its entirety.

Alternatively, the antibodies are obtained by screening a library that contains antibody or antigen binding domain sequences for binding to the target antigen. This library is prepared, e.g., in bacteriophage as protein or peptide fusions to a bacteriophage coat protein that is expressed on the surface of assembled phage particles and the encoding DNA sequences contained within the phage particles (i.e., “phage displayed library”).

Hybridomas resulting from myeloma/B cell fusions are then screened for reactivity to the target antigen. Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

Although not strictly impossible, the serendipitous identification of different antibodies having the same heavy chain variable domain but directed against different antigens is highly unlikely. Indeed, in most cases the heavy chain contributes largely to the antigen binding surface and is also the most variable in sequence. In particular the CDR3 on the heavy chain is the most diverse CDR in sequence, length and structure. Thus, two antibodies specific for different antigens will almost invariably carry different heavy chain variable domains.

The methods disclosed in co-pending application WO 2012/023053 overcomes this limitation and greatly facilitates the isolation of antibodies having the same heavy chain variable domain by the use of antibody libraries in which the heavy chain variable domain is the same for all the library members and thus the diversity is confined to the light chain variable domain. Such libraries are described, for example, in co-pending applications WO 2010/135558 and WO 2011/084255, each of which is hereby incorporated by reference in its entirety. However, as the light chain variable domain is expressed in conjunction with the heavy variable domain, both domains can contribute to antigen binding. To further facilitate the process, antibody libraries containing the same heavy chain variable domain and either a diversity of Lambda variable light chains or Kappa variable light chains can be used in parallel for in vitro selection of antibodies against different antigens. This approach enables the identification of two antibodies having a common heavy chain but one carrying a Lambda light chain variable domain and the other a Kappa light chain variable domain that can be used as building blocks for the generation of a bispecific antibody in the full immunoglobulin format of the invention. The bispecific antibodies of the invention can be of different Isotypes and their Fe portion can be modified in order to alter the bind properties to different Fc receptors and in this way modify the effectors functions of the antibody as well as it pharmacokinetic properties. Numerous methods for the modification of the Fe portion have been described and are applicable to antibodies of the invention. (see for example Strohl, WR Curr Opin Biotechnol 2009 (6):685-91; U.S. Pat. No. 6,528,624; PCT/US2009/0191199 filed Jan. 9, 2009). The methods of the invention can also be used to generate bispecific antibodies and antibody mixtures in a F(ab′)2 format that lacks the Fc portion.

The common heavy chain and two different light chains are co-expressed into a single cell to allow for the assembly of a bispecific antibody of the invention. If all the polypeptides get expressed at the same level and get assembled equally well to form an immunoglobulin molecule then the ratio of monospecific (same light chains) and bispecific (two different light chains) should be 50%. However, it is likely that different light chains are expressed at different levels and/or do not assemble with the same efficiency. Therefore, a means to modulate the relative expression of the different polypeptides is used to compensate for their intrinsic expression characteristics or different propensities to assemble with the common heavy chain. This modulation can be achieved via promoter strength, the use of internal ribosome entry sites (IRES) featuring different efficiencies or other types of regulatory elements that can act at transcriptional or translational levels as well as acting on mRNA stability. Different promoters of different strength could include CMV (Immediate-early Cytomegalovirus virus promoter); EF1-1α (Human elongation factor 1α-subunit promoter); Ube (Human ubiquitin C promoter); SV40 (Simian virus 40 promoter). Different RES have also been described from mammalian and viral origin. (See e.g., Hellen C U and Sarnow P. Genes Dev 2001 15: 1593-612). These IRES can greatly differ in their length and ribosome recruiting efficiency. Furthermore, it is possible to further tune the activity by introducing multiple copies of an IRES (Stephen et al. 2000 Proc Natl Acad Sci USA 97: 1536-1541). The modulation of the expression can also be achieved by multiple sequential transfections of cells to increase the copy number of individual genes expressing one or the other light chain and thus modify their relative expressions. The Examples provided herein demonstrate that controlling the relative expression of the different chains is critical for maximizing the assembly and overall yield of the bispecific antibody.

The co-expression of the heavy chain and two light chains generates a mixture of three different antibodies into the cell culture supernatant: two monospecific bivalent antibodies and one bispecific bivalent antibody. The latter has to be purified from the mixture to obtain the molecule of interest. The method described herein greatly facilitates this purification procedure by the use of affinity chromatography media that specifically interact with the Kappa or Lambda light chain constant domains such as the CaptureSelect Fab Kappa and CaptureSelect Fab Lambda affinity matrices (BAC BV, Holland). This multi-step affinity chromatography purification approach is efficient and generally applicable to antibodies of the invention. This is in sharp contrast to specific purification methods that have to be developed and optimized for each bispecific antibodies derived from quadromas or other cell lines expressing antibody mixtures. Indeed, if the biochemical characteristics of the different antibodies in the mixtures are similar, their separation using standard chromatography technique such as ion exchange chromatography can be challenging or not possible at all.

Other suitable purification methods include those disclosed in co-pending application PCT/IB2012/003028, fled on Oct. 19, 2012, published as WO2013/088259, the contents of which are hereby incorporated by reference in their entirety.

In other embodiments of producing bispecific antibodies, antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface includes at least a pail of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al, J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g, CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate antibodies are also within the scope of the present invention. Heteroconjugate antibodies are composed of two covalently joined antibodies. Such antibodies have, for example, been proposed to target immune system cells to unwanted cells (see U.S. Pat. No. 4,676,980), and for treatment of HIV infection (see WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It can be desirable to modify the antibody of the invention with respect to effector function, so as to enhance, e.g., the effectiveness of the antibody in treating cancer and/or other diseases and disorders associated with aberrant CD28 expression and/or activity. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)).

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomnycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of possible moieties can be coupled to the resultant antibodies of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987).

Preferred linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Particularly preferred linkers include: (i) EDC (i-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propionamide]hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NIS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NIS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al., Proc. Natl Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction.

### Methods of Use

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the invention, which include an antibody of the invention, are used to treat or alleviate a symptom associated with a cancer, such as, by way of non-limiting example, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma The present invention also provides methods of treating or alleviating a symptom associated with a cancer. A therapeutic regimen is carried out by identifying a subject, e.g., a human patient suffering from (or at risk of developing) a cancer, using standard methods.

Efficaciousness of treatment is determined in association with any known method for diagnosing or treating the particular immune-related disorder. Alleviation of one or more symptoms of the immune-related disorder indicates that the antibody confers a clinical benefit.

Methods for the screening of antibodies that possess the desired specificity include, but are not limited to, enzyme linked immunosorbent assay (ELISA) and other immunologically mediated techniques known within the art.

Antibodies directed against a target such as CD28, PD-L1, or a combination thereof (or a fragment thereof), may be used in methods known within the an relating to the localization and/or quantitation of these targets, e.g., for use in measuring levels of these targets within appropriate physiological samples, for use in diagnostic methods, for use in imaging the protein, and the like). In a given embodiment, antibodies specific any of these targets, or derivative, fragment, analog or homolog thereof, that contain the antibody derived antigen binding domain, are utilized as pharmacologically active compounds (referred to hereinafter as “Therapeutics”).

An antibody of the invention can be used to isolate a particular target using standard techniques, such as immunoaffinity, chromatography or immunoprecipitation Antibodies of the invention (or a fragment thereof) can be used diagnostically to monitor protein levels in tissue as part of a clinical testing procedure, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocynate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies of the invention, including polyclonal, monoclonal, humanized and fully human antibodies, may be used as therapeutic agents. Such agents will generally be employed to treat or prevent a disease or pathology associated with aberrant expression or activation of a given target in a subject. An antibody preparation, preferably one having high specificity and high affinity for its target antigen, is administered to the subject and will generally have an effect due to its binding with the target. Administration of the antibody may abrogate or inhibit or interfere with the signaling function of the target. Administration of the antibody may abrogate or inhibit or interfere with the binding of the target with an endogenous ligand to which it naturally binds. Administration of the antibody may activate, or stimulate or enhance the signaling function of the target.

A therapeutically effective amount of an antibody of the invention relates generally to the amount needed to achieve a therapeutic objective. As noted above, this may be a binding interaction between the antibody and its target antigen that, in certain cases, interferes with the functioning of the target. In some embodiments, administration of the antibody may activate, or stimulate or enhance the signaling function of the target. In some embodiments, the antibody may abrogate or inhibit or interfere with the binding of the target with an endogenous ligand to which it naturally binds and may activate, or stimulate or enhance the signaling function of another target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Antibodies or a fragment thereof of the invention can be administered for the treatment of a variety of diseases and disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harvood Academic Publishers, Langhorne, Pa., 1994; and Peptide And Protein Drug Delivery (Advances In Parenteral Sciences, Vol. 4), 1991, M. Dekker, New York.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

An antibody according to the invention can be used as an agent for detecting the presence of a given target (or a protein fragment thereof) in a sample. In some embodiments, the antibody contains a detectable label. Antibodies are polyclonal, or more preferably, monoclonal. An intact antibody, or a fragment thereof (e.g., F_{ab}, scFv, or F_{(ab)2}) is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph, That is, the detection method of the invention can be used to detect an analyte mRNA, protein, or genomic DNA in a biological sample in vitro as well as in vivo. For example, in intro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N J, 1995; “Immunoassay”, E. Diamandis and T. Christopoulus, Academic Press, Inc., Sari Diego, C A, 1996; and “Practice and Theory of Enzyme Immunoassays” P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

### Pharmaceutical Compositions

The antibodies of the invention (also referred to herein as “active compounds”), and derivatives, fragments, analogs and homologs thereof, can be incorporated into pharmaceutical compositions suitable for administration. Such compositions typically comprise the antibody and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g, with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention will be further described in the following examples, which do not limit the scope of the invention described in the claims.

### EXAMPLES

### Example 1: Expression and Purification of Bispecific Antibodies Carrying a Lambda and a Kappa Light Chain

The simultaneous expression of one heavy chain and two lights chain in the same cell can lead to the assembly of three different antibodies. Simultaneous expression can be achieved in different ways such as that the transfection of multiple vectors expressing one of the chains to be co-expressed or by using vectors that drive multiple gene expression.

Here, the two light chains were cloned into the vector pNovi κHλ that was previously generated to allow for the co-expression of one heavy chain, one Kappa light chain and one Lambda light chain as described in US20120184716 and WO2012023053, each of which is hereby incorporated by reference in its entirety. The expression of the three genes is driven by human cytomegalovirus promoters (hCMV) and the vector also contains a glutamine synthetase gene (GS) that enables the selection and establishment of stable cell lines. The common VH and the VL genes of the anti-CD28 IgG and of the anti-PD-L11 IgG were cloned in the vector pNovi κHλ, for transient expression in mammalian cells. Expi293 cells were cultured in suspension in an appropriate Erlenmeyer flask with suitable number of cells and culture medium volume. Plasmid DNA was transfected into Expi293 cells using PEI. Antibody concentration in the supernatant of transfected cells was measured during the production using an Octet RED96, According to antibody concentration, supernatants were harvested 5 to 7 days after transfection and clarified by filtration after addition of diatomaceous earth (Sartorius). The purification was based on a three-step purification process. First, the CaptureSelect™ FcXL affinity matrix (Thermo Fisher Scientific) was washed with PBS and then added in the clarified supernatant. After incubation overnight at +4° C. and 20 rpm, supernatants were centrifuged at 2000 g for 10 min., flow through was stored and resin were washed twice with PBS. Then, the resin was transferred on Amicon Pro columns and a solution containing 50 mM glycine at pH 3.5 was used for elution. Several elution fractions were generated, neutralized with Tris-HCl pH7.4 and pooled. The pool containing total human IgGs (the bispecific and the two monospecific antibodies) was quantified using a Nanodrop spectrophotometer (NanoDrop Technologies). A small aliquot was stored for further analysis and the remaining sample was incubated for 30 min at RT and 20 rpm with the appropriate volume of CaptureSelect™ KappaXL affinity matrix (Thermo Fisher Scientific). Resin recovery and wash, elution and neutralization steps were performed as described above. The last affinity purification step was performed using the CaptureSelect™ lambda Fab affinity matrix (Thermo Fisher Scientific) applying the same process as for the kappa purification step. Alternatively, the purification was based on a two-step purification process, where only the CaptureSelect™ KappaXL affinity matrix and the CaptureSelect™ lambda Fab affinity matrix were used. All elution fractions were pooled and desalted against His-NaCl pH 6.0 formulation buffer using 50 kDa Amicon Ultra centrifugal filter units (Merck Millipore). The final product was quantified using the Nanodrop.

Purified bispecific antibodies were analyzed by electrophoresis in denaturing and reducing conditions using an Agilent 2100 Bioanalyzer with the Protein 80 kit as described by the manufacturer (Agilent Technologies). The aggregate level was determined by SEC-UPLC. All samples were tested for endotoxin contamination using the Limulus Amebocyte Lysate test (LAL; Charles River Laboratories). Table 2 summarizes the PD-L1xCD28 X bodies generated.

TABLE 2
Nomenclature And Chains Used To Generate The Panel
Of κλ-Bodies Of The Invention. AI Denotes
Anti Human CD28 Arms, S Denotes Anti Human PD-L1
Arms. “/N” denotes a heavy chain with LALAPA mutation.

| | Kappa | Lambda | |
|---|---|---|---|
| BsAb name | light chain | light chain | Heavy chain |
| AI3S79/N | AI3 | S79 | Common VH with |
| AI13S79/N | AI13 | S79 | LALAPA mutations |

### Example 2: In Vitro Characterization of Pd-L1xCD28 Bispecific Antibodies

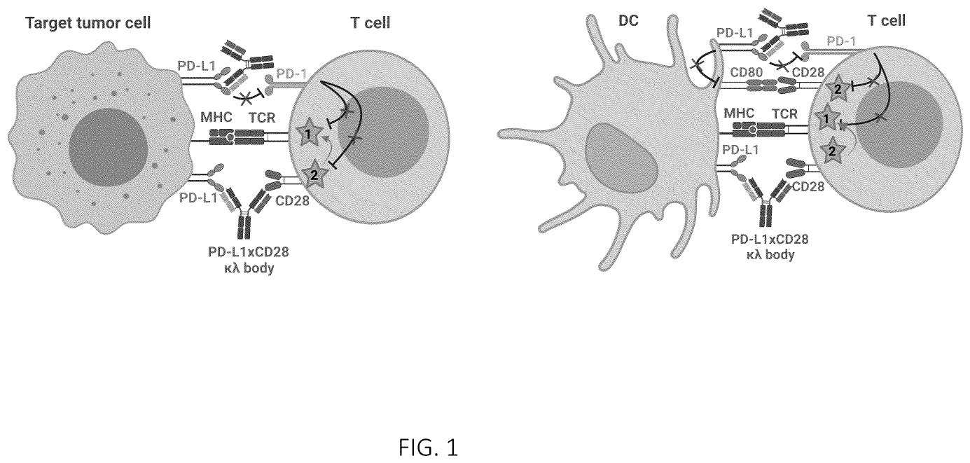

To demonstrate the binding of PD-L1xCD28 κλ bodies to target cells (FIG. 1), a series of experiments based on flow cytometry was performed.

Examples of cells that can be used include PD-L1-positive cell lines such as the pancreatic adenocarcinoma epithelial cell line HPAC, CD28-positive cell lines such as the leukemic Jurkat T cells as well as PD-L1 and CD28 double negative cell lines, such as the leukemic TIB153 cells.

Cells were harvested, checked for viability and counted. 200′000 cells were incubated for 15 minutes at 4° C. with increasing concentrations of the antibodies diluted in FACS buffer (PBS 2% BSA, 0.1% NaN3). Cells were washed twice with cold FACS buffer and re-incubated for further 15 minutes at 4′C with a suitable anti-human IgG secondary antibody. Cells were washed twice with cold FACS buffer and resuspended in 150 μl FACS buffer with a compatible viability marker. Binding of antibodies to living cells was measured by flow cytometry using a Cytoflex Platform (Beckman Coulter). Data was analyzed with FlowJo™ v10 software (BD Life Sciences) and dose-response binding curves were drawn using GiraphPad Prism 9 software.

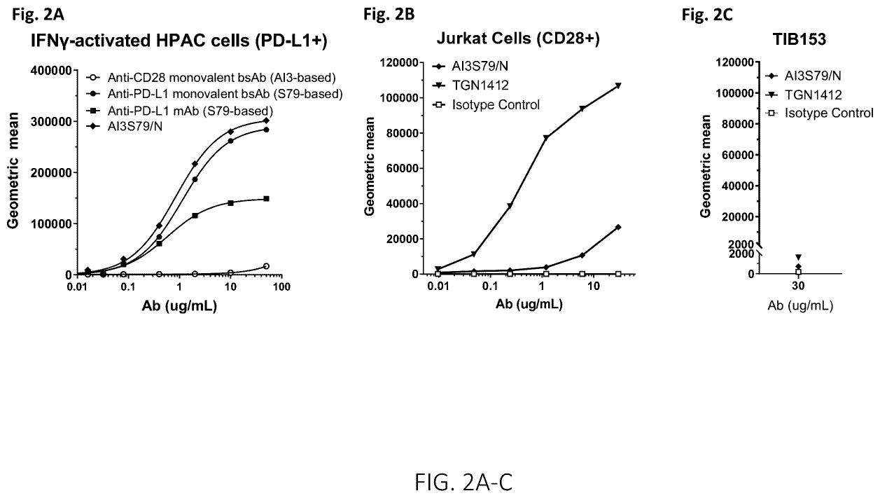

Binding curves of the exemplary PD-L1xCD28 bsAb AI3S9/N of the invention obtained using HPAC, Jurkat, and TIB153 cells are shown in FIGS. 2A, B, and C, respectively.

AI3S79/N is binding to cells which express either PD-L1 (FIG. 2A) or CD28 (FIG. 213). The absence of binding signal on TIB-153 suggests that all binding arms of the invention are specific for the designated target (FIG. 2C).

The affinities of AI3S79/N for (D28, PD-L1, Fe gamma Receptors (FcγRs) and neonatal Fe Receptor (FcRn) from human, cynomolgus and mouse species were determined by Octet® surface plasmon resonance (Tables 3, 4, 5, 6 and 7).

TABLE 3
Affinity of AI3S79/N for human, cynomolgus
and mouse CD28 and PD-L1 targets

| | Species | | |
|---|---|---|---|
| Target | Human | Cynomolgus | Mouse |

| | | |
|---|---|---|
| CD28 | 54 ± 3 | 11100 ± 714 |

| | | | |
|---|---|---|---|
| PD-L1 | 0.3 ± 0.1 | 1.8 ± 0.2 | 0.79 ± 0.06 |

KD (nM) ± SD (nM), n = 2

The extracellular domains of human and cynomolgus CD28 are 100% identical, hence the equivalent KID value of AUS79/N for human and cynomolgus CD28 (Table 3). This data showed that AI3S79/N is cross-reactive on cynomolgus CD28 (KD ˜54 nM on both human and cynomolgus CD28) and PD-L1 (KD 0.3 nM on human and ˜1.8 nM on cynomolgus). The anti-PD-L1 arm of AI3S79/N is also cross-reactive on mouse PD-L1 (KD ˜0.3 nM on human and ˜0.79 nM on mouse). However, the anti-CD28 arm of AI3S79/N is not cross-reactive on mouse CD28. Indeed, it showed a poor affinity on mouse CD28 and is thus considered not cross-reactive.

AI3S79/N presents comparable affinities on human and cynomolgus FcRn (KD ˜7.1 nM on human and ˜8.6 nM on cynomolgus) as shown in Table 4.

TABLE 4
Affinity of AI3S79/N for human, cynomolgus and mouse FcRn

| | Species | | |
|---|---|---|---|
| FcR | Human | Cynomolgus | Mouse |
| FcRn | 7.1 ± 0.5 | 8.6 ± 0.6 | 0.06 ± 0.03 |

KD (nM) ± SD (nM), n = 2

AI3S79/N binds to human CD64 with a poor affinity (KD ˜804 nM) and does not bind to other human FcγRs (CD32a R167, CD32a-H167, CD32b, CD16a V158, CD16a F158, CD16b) as shown in Table 5.

TABLE 5
Affinity of AI3S79/N for human FcγRs

| | hCD32a | hCD32a | | hCD16a | hCD16a | hCD16b |
|---|---|---|---|---|---|---|
| hCD64 | R167 | H167 | hCD32b | V158 | F158 | NA2 |
| 804 | No binding | No binding | No binding | No binding | No binding | No binding |

h: human; KD (nM) ± SD (nM), n = 1 (highest tested concentration: 2000 nM)

The LALAPA mutations introduced in AI3S79/N made this molecule Fe silent. The absence of binding of AI3S79/N to FcγRs prevents Fc-mediated effector functions (i.e ADCP or ADCC).

ADS79/N binds to cynomolgus CD64 with a poor affinity (KD ˜183 nM) and does not bind to other cynomolgus FcγRs (CD32a, CD32b, CD16) as shown in Table 6. As in human, it is expected that the LALAPA mutations in AI3S79/N prevent Fc-mediated effector functions (i.e. ADCP, ADCC and CDC) in cynomolgus monkeys.

TABLE 6
Affinity of AI3S79/N for cynomolgus FcγRs

| cyCD64 | cyCD32a | cyCD32b | cyCD16 |
|---|---|---|---|
| 183 | No binding | No binding | No binding |

cy: cynomolgus;
KD (nM) ± SD (nM), n = 1 (highest tested concentration: 2000 nM)

AI3S79/N does not bind to any of the mouse FcγRs tested (C)64, CD32b, CD16), as shown in Table 7.

TABLE 7
Affinity of AI3S79/N for mouse FcγRs

| mCD64 | mCD32b | mCD16 |
|---|---|---|
| No binding | No binding | No binding |

KD (nM) ± SD (nM), n = 1 (highest tested concentration: 2000 nM)

### Example 3: PD-1/PD-L1 Blockade Bioassay

The capacity of AI3S79/N to block the PD-1/PD-L1 interaction was assessed using a PD-1/PD-L1 blockade bioassay, which is a biologically relevant MOA-based assay that measure the potency of antibodies designed to block the PD-1/PD-L1 interaction (Pronega, J1250).

The kit consists of two cell lines: (1) an artificial Antigen Presenting Cell (aAPC) based on engineered CHO-K1 cells expressing at the cell surface both PD-L1 and a protein designed to activate cognate TCRs in an antigen-independent manner; and (2) Jurkat T cells stably expressing human PD-1 and NFAT-induced luciferase. When the two cell types are co-cultured, the PD-1/PD-L1 interaction inhibits TCR signaling and NFAT-mediated luciferase activity. Addition of an antibody that blocks either PD-1 or PD-L1 releases the inhibitory signal and results in TCR signaling and NFAT-mediated luciferase activity.

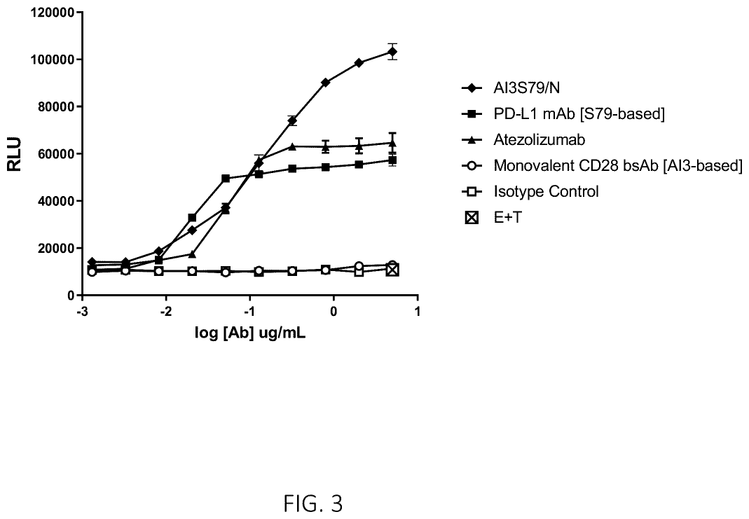

As shown in FIG. 3, because the reporter cells also express CD28 at the cell surface, AI3S79/N not only triggers the luminescence via the blockade of PD-1/PD-L1 engagement, but further boost it via CD28 costimulation, resulting in higher RLU signals than simple PD-(L)1 blockers (i.e., Atezolizumab or the bivalent PD-L1 mAb based on the anti-PD-L1 arm S79). Because of the lack of a PD-(L)1 blocking arm, the CD28 monovalent bsAbs is unable to induce the reporter system on its own, triggering no luminescence signal.

This reporter assay confirms that, in presence of T cell signal 1 (provided by the aAPC) and PD-L-1, the PD-L1xCD28 bispecific antibodies of the invention can enhance T cell response by inhibiting the PD-1/PD-11 interaction, while further delivering co-stimulatory signal 2 to T cells.

### Example 4: T-Cell Dependent Cellular Cytotoxicity (TDCC) Mediated by Pd-L1xCD28 Bispecific Antibodies

### TDCC of PD-1 and CEA Double-Positive Cell Lines

The T-cell dependent cellular cytotoxicity (TDCC) of a PD-L1/CEA double positive cell line induced by the PD-L1xCD28 bispecific antibodies of the invention was assessed in combination with a CEAxCD3 bsAb using human PBMCs as effector cells.

Target cells were detached with trypsin or cell dissociation solution after two washes with PBS. After a centrifugation step, cells were resuspended in assay media, adjusted to the needed concentration, and plated in 96-well plates.

Effector cells were human peripheral blood mononuclear cells (PBMCs) isolated from buffy coats derived from healthy human donors using SepMate™ Tubes (Stemcell Technologies) with Lymphoprep™ buffer (Stemcell Technologies).

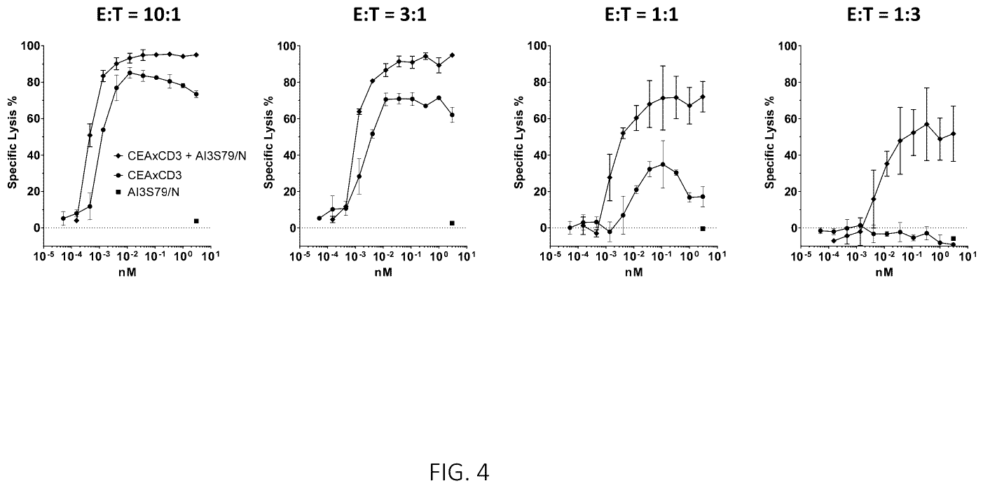

For the TDCC assay, PBMCs were added to target cells at different final E:T ratios (10:1, 3:1, 1:1 and 1:3). A dose range of CEAxCD3 and a fixed dose of the PD-L1xCD28 antibodies of the invention (2.5 μg/mL) were added to the pre-plated target and effector cells. As negative control, single-agent AI3S79/N was used (no CEAxCD3=no T cell signal 1). Target cell killing is assessed after 6 days of incubation at 37° C., 5% CO2 by quantifying the number of viable adherent cells in culture using Promega's CellTiter-Glo® (G7570). TDCC curves for each ET ratio (FIG. 4) were plotted using GraphPad Prism 9.

PD-L1xCD28 bsAbs synergized with CEAxCD3 bsAb to kill PD-L1/CEA double-positive HPAC target cells, especially at lower E:T ratio (FIG. 4, right panels). If at optimal E:T ratio (i.e., 10:1) the CD3 bsAb is capable of consistently inducing target cell killing, with decreasing E:T ratio its activity declined, up to the point that, as single agent, it was totally unable to induce killing of target cells (i.e. at a E:T ratio of 1:3). However, along the E:T ratio gradient, the addition of AI3S79/N strengthened the activity of the CD3 bsAb, with specific lysis as high as 50% observed even at the unfavorable 1:3 E:T ratio. Importantly, in the absence of CEAxCD3, no killing was induced by the PD-L1xCD28 bsAb alone, highlighting the importance of primary T cell stimulation (signal 1) for the activity of PD-L1xCD28 bsAbs.

Up-Regulation of T-Cell Activation Markers Upon Killing of PD-L1/CEA-Expressing Tumor Cells Induced by the Combination of CEAxCD3 and PD-L1xCD28 bsAbs.

Killing of CEA-positive tumor cells induced by CEAxCD3 bsAbs is based on T cell activation. The activation state of a T cell can be further increased by CD28 co-stimulation. The capacity of PD-L1xCD28 κλ. bodies to enhance T cell activation in presence of a proper signal 1 was thus quantified by flow cytometry using antibodies recognizing specific T cell activation markers such as CD25 (late activation marker).

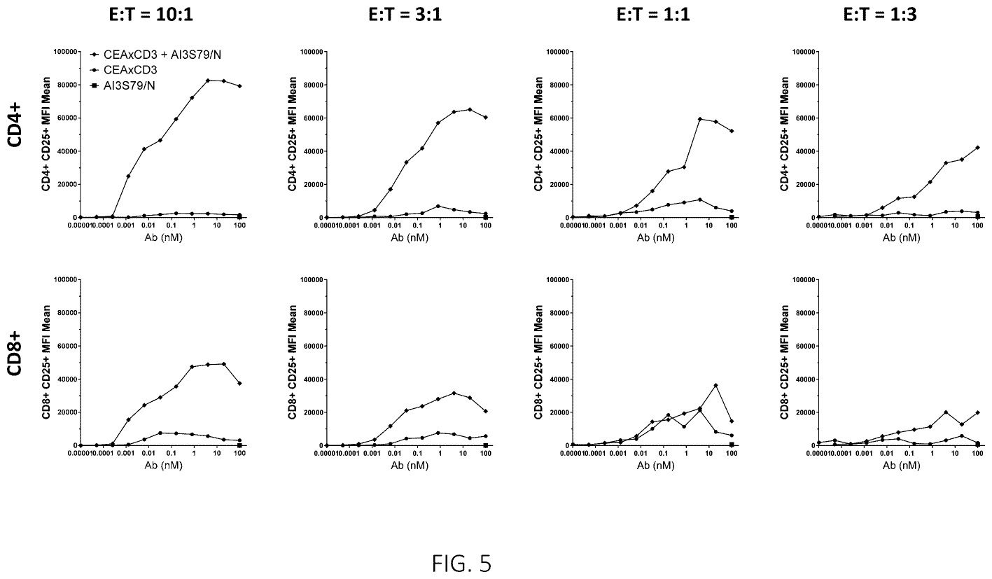

To assess the activation state of T cells at the end of a killing assay (detailed in example 4a), the following procedure was applied: floating cells (which include both CD4+ and CD-8+ T-cells) were transferred into a new V-bottom 96-well plates. The supernatant was removed by centrifugation and cells were washed twice with cold FACS buffer (PBS 2% BSA, 0.1% NaN3) before being incubated for 15 minutes at 4° C. with Fc-block reagent (BD Biosciences). After two washing with FACS buffer, cells were incubated for 15 minutes at 4° C. with following antibodies: anti-CD8-PerCP-Cy5.5 (BioLegend), anti-CD25-PE (BioLegend) and anti-CD4-APC (ThermoFisher). Cells were washed and analyzed by flow cytometry using a Cytoflex Platform (Beckman Coulter). Data was analyzed using FlowJo™ v10 software (BD Life Sciences). Results of the quantification of T cell activation of the TDCC experiment shown in FIG. 4 are shown in FIG. 5.

T cell activation was measured by quantifying the late activation marker CD25 at the surface of both CD4+ and CD8+ T cells (FIG. 5 top and bottom row, respectively) at the different E:T ratios. Compared to the single treatment with CEAxCD3, the combination of CEAxCD3 and AI3S79/N activated both CD4+ and CD8+ T cells to a greater extent, with much brighter CD25 staining for the combination treatments (up to 47 times higher MFI signal for the combination compared to the CD3 bsAb alone). The synergy between CD3 and CD28 bsAbs is best observed at high E:T ratios, but even at the unfavorable 1:3 E:T ratio, T cells displayed brighter CD25 staining (13-fold higher MFI for the combo compared to the CD3 bsAb alone at the highest antibody concentration).

Effect of CD28 Costimulation Mediated by PD-L1xCD28 bsAbs on T Cell Proliferation in Presence of a C4xCD3 bsAb.

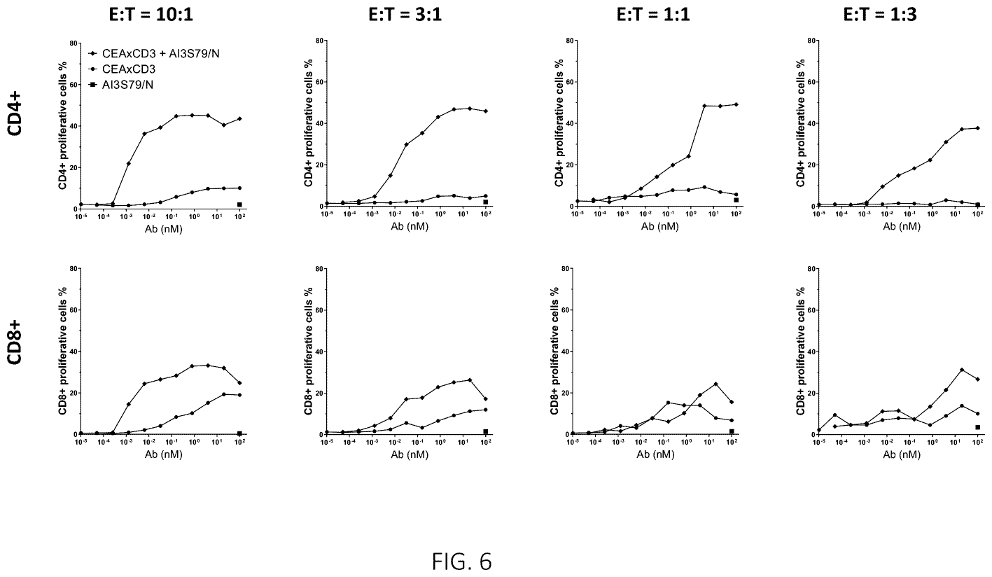

AI3S79/N was analyzed for its capability to enhance the effects of CEAxCD3 in term of induction of T cell proliferation in the presence of PD-L1/CEA-positive tumor target cells. Freshly isolated human PBMCs were stained with CellTrace Violet Cell Proliferation Kit (ThermoFischer Scientific) according to the manufacturer's instructions, washed and co-cultured with target cells at different E:T ratios, in the presence of a dose range of CEAxCD3 and 2.5 μg/mL fixed dose of PD-L1xCD28 bsAb. Following the co-culture, the effector cells were harvested, washed, stained with a suitable viability marker to exclude dead cells and with anti-CD4-APC (ThermoFischer, 17-0049-41) and anti-CD8-PerCP-Cy5.5 (BioLegend, 301032) to identify the populations of interest. The proliferation rate of T cells was calculated by measuring the levels of CellTrace Violet staining intensity on living CD4+ or CD8+ T cells by flow cytometry using a CytoFLEX (Beckman Coulter). Data was evaluated by FlowJo software and plotted using GraphPad Prism (FIG. 6)

The percentage of proliferative CD4-+ and CD8+ T cells at the different E:T ratios is shown in the top and bottom row of FIG. 6, respectively. The capacity of a CEAxCD3 bsAb to induce T cell proliferation was enhanced by the addition of AI3S79/N, with CD4 T cells that were more positively influenced by the combination treatments than CD8 T cells. As per the T cell activation state, T cell proliferation is best observed at high E:T ratios, but even at the unfavorable 1:3 E:T ratio, T cells displayed higher proliferation potential when treated with the combination (up to 35-fold higher percentage of proliferative T cells for the combo compared to the CD3 bsAb alone).

### Example 5: In Vivo Efficacy Study of AI3S79/N in the Treatment of MC38-HPD-L1 Engrafted in CD28 HuGEMM Mice

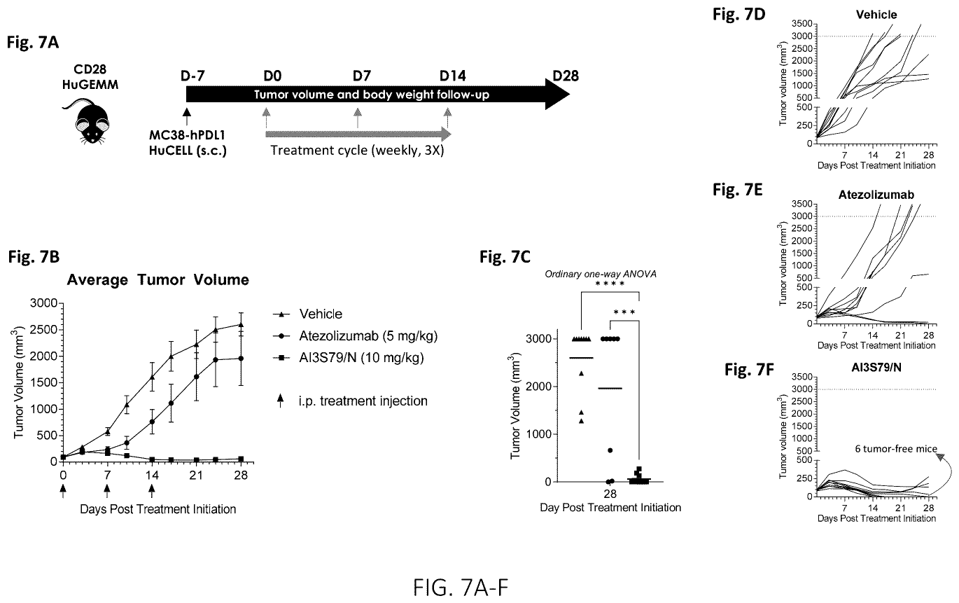

This efficacy study aimed at assessing the anti-tumoral effect of AI3S79/N as a single therapy. The experimental design is summarized in FIG. 7A. Briefly: MC38-hPD-L1 HuCELL (MC38 cells constitutively expressing human PD-L1 at the cell surface; Crown Bioscience) were engrafted subcutaneously in CD28 HuGEMM mice (immunocompetent chimeric mouse model engineered to express humanized CD28 instead of mouse CD28; Crown Bioscience). Mice were randomized when the average tumor volume was close to 100 mm3 (7 days post engraftment) and treated weekly for a total of 3 injections (FIG. 7A), with: vehicle, Atezolizumab [5 mg/kg, bivalent anti-PD-L1 antibody] or AI3S79/N [10 mg/kg, PD-L1xCD28, monovalent engagement of each target]. Tumors were measured twice weekly by digital caliper until the endpoint of the experiment (tumor volume=3000 mm3). Tumor volume was calculated using the formula (length×width2)×0.5.

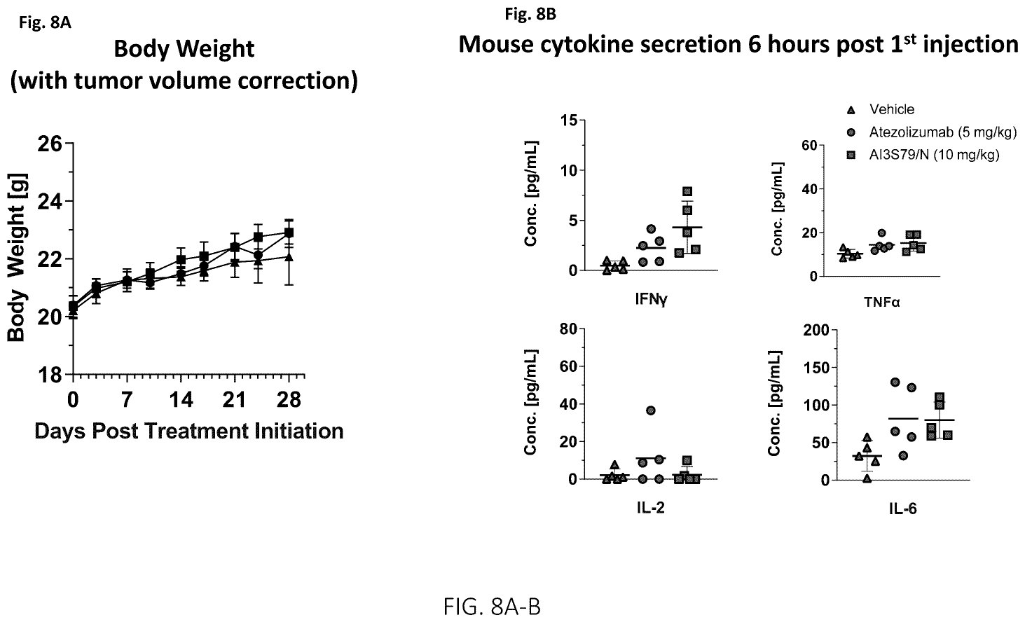

Contrary to Atezolizumab, which only resulted in limited tumor growth inhibition compared to the vehicle control, AI3S79/N led to tumor regression in all mice (FIG. 7B). Tumor volume comparison at study termination (day 28 post treatment initiation) highlights a statistically stronger anti-tumor activity of AI3S79/N compared to Atezolizumab (FIG. 7C). The tumor growth kinetic of individual mice is shown for vehicle (FIG. 7D), Atezolizunab (FIG. 7E) and AI3S79/N (FIG. 7F). Of note, 6 out of 10 mice treated with AI3S79/N were considered as tumor-free at study termination (day 28), with two additional mice that had small regressing tumors. Normal weight gain and no clinical signs of toxicity were observed following treatment cycle (FIG. 8A). Cytokine secretion was measured 6 hours post 1st injection of either vehicle, atezolizumab (5 mg/kg) or AI3S79/N (10 mg/kg) (FIG. 8B) and low cytokine concentrations were detected in mice treated with AI3S79/N, in line with those measured for Atezolizumab-treated mice, suggesting no overactivation of the immune system. Overall, AI3S79/N was well tolerated in immunocompetent huCD28 mice in which AI3S79/N acts as a fully mouse cross-reactive antibody.

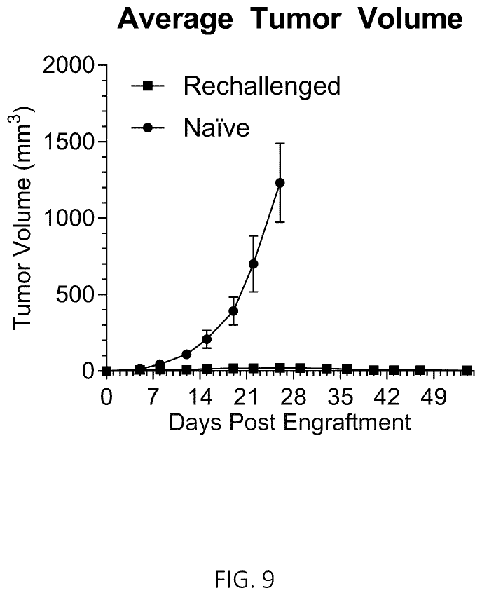

Because the majority of AI3S79/N treated mice survived the primary tumor challenge with MC38-huPD-L1 (8/10 mice) (experimental protocol shown in FIG. 7A), survivals were rechallenged with MC38 WT cells on the opposite flank on day 59 (FIG. 9). Contrary to naïve mice, which all succumbed to the MC38 WT tumor, none of the rechallenged mice developed a tumor, suggesting that AI3S79/N induced immunological memory against MC38 cells.

### Example 6: AI3S79/N is not a CD28 Superagonist

The AI3 anti-CD28 arm was already shown not to be a superagonist in WO2023170474. To exclude superagonist activity in the context of the bsAb of the present invention, AI3S79/N was tested in two distinct in vitro safety assays for its capacity to induce T cell proliferation or T cell-mediated cytokine release in the absence of signal 1.

### Wet and Dry Plate Coating T Cell Proliferation Assay (Stebbings Assay)

Method is adapted from Stebbings et al (2007). Briefly: 96-well polypropylene plates were coated overnight with antibodies diluted to 10 μg/ml in PBS, either at 4° C. with 100 μl of antibody solution (wet coating) or at room temperature, unsealed and in a class II laminar flow cabinet (to allow for the evaporation of the buffer=dry coating), with 50 μL of antibody solution. Following either coating procedure, plates were washed twice with PBS. In parallel, PBMCs isolated from buffy coat obtained from healthy donors were stained with CellTrace Violet Cell Proliferation Kit (ThermoFischer Scientific) according to the manufacturer's instructions. 100′000 stained PBMC cells were added to the 96-well plate in a final volume of 200 μL/well and incubated at 37° C.+5% CO2 for 6 days. Cells were then harvested and stained for flow cytometry assessment using anti-CD4-APC (ThermoFischer, 17-0049-41) and anti-CD8-PerCP-Cy5.5 (BioLegend, 301032) as detailed in example 2 and 4c. The proliferation rate of living CD4+ and CD8+ T cells was calculated by measuring the levels of CellTrace Violet staining by flow cytometry using a CytoFLEX (Beckman Coulter) and results were evaluated by Flo Jo software for both coating procedures. The anti-CD3 antibody OKT3 and the CD28 SA antibody TGN1412 served as positive controls, while the background proliferation rate of T cells was determined in presence of an isotype control antibody.

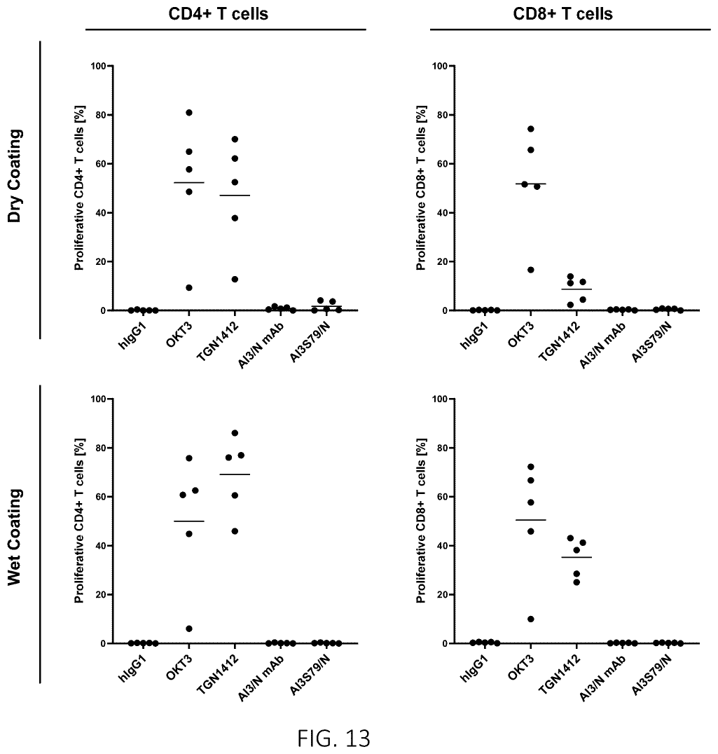

As shown in FIG. 13, the TGN1412 analogue induced T cell proliferation of both CD4+ and CD8 subtypes, in both wet and dry coating conditions, confirming its superagonistic feature. In contrast, the AI3 anti-CD28 arm induced no proliferation of resting CD4+ and CD8+ T cells under both experimental conditions (wet and dry coating), neither as a monoclonal antibody (mAb AI3/N), nor as part of a PD-L1xCD28 bsAb of the invention (AI3S79/N).

### Preculture of PBMCs at High Cell Density Fir Increased Sensitivity of T-Cell Responses (Restore Protocol)

To confirm the lack of superagonism of AI3N79/N in a more physiologically relevant setting, AI3S79/N was tested according to the “RESTORE” protocol (Römer et al. 2011). PBMC from healthy donors were first pre-cultured at high density (HDP) to induce functional maturation of both monocytes and T cells, and then cultured at normal density in presence of soluble anti bodies.

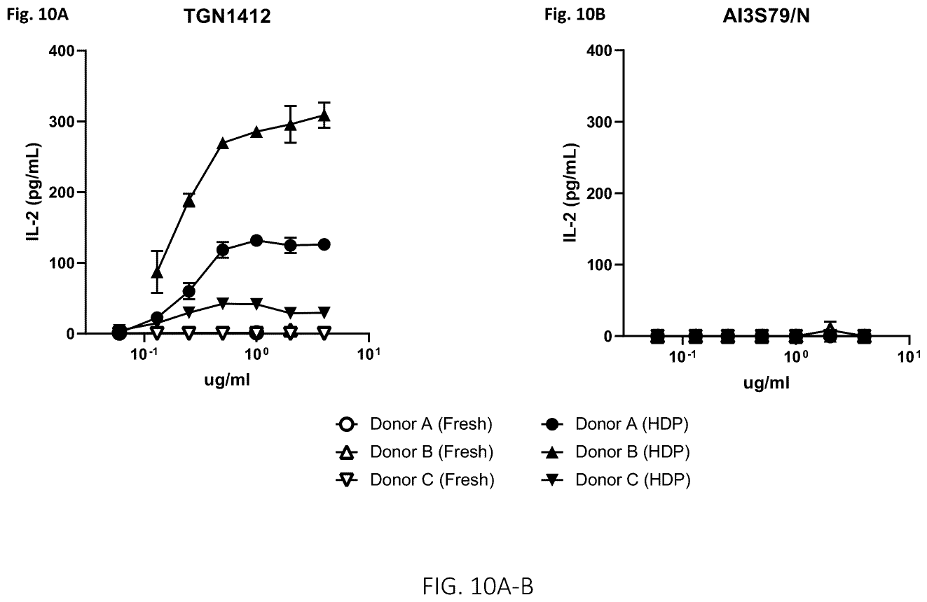

Under such conditions, the TGN1412 analogue induced dose-dependent secretion of IL-2 with all three donors tested, although at different extent, reflecting donor variability (FIG. 10A). As expected, the superagonistic activity of TGN1412 does not emerge if PBMC are not subjected to the preculture step at high density (fresh PBMC). Importantly, AI3S79/N did not induce any secretion of IL-2, not even when added to HDP PBMCs (FIG. 10B.)

### Example 7: T Cell Activation in a Mixed Lymphocyte Reaction in Presence of a T Cell Superantigen

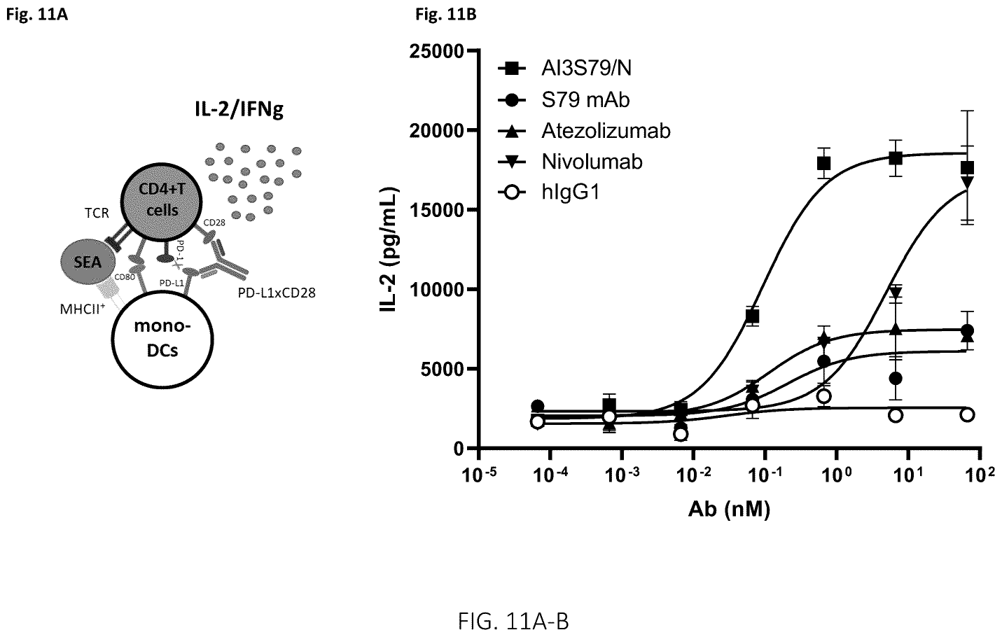

A mixed lymphocyte reaction (MLR) is an in vitro assay in which immune cells from two individuals are co-cultured to trigger the ‘non-self’ recognition required for allogeneic T cell activation and proliferation. In this assay, immune checkpoint inhibitors (ICI) such as anti-PD1 or anti-PD-L1 nAbs enhance the MLR, as measured by an increase in cytokine secretion, To assess the capacity of AI3′S79/N to enhance T cell response in presence of proper stimuli, a variant of a MLR where CD4+ T cells (responder) and monocyte-derived DCs (stimulator) are cocultured in presence of the Staphylococcal aureus T cell superantigen SEA was developed (FIG. 11A).

As expected, both Atezolizumab and Nivolumab enhance the IL-2 secretion in such MLR assay (FIG. 11B). The profile of the two antibodies is not the same and may reflect the fact that Atezolizumab only blocks PD-1/PD-1 interaction, while Nivolumab, by binding to PD-1, could block both PD-L1/PD-1 and PD-L2/PD-1 interactions. S79 mAb, a bivalent monoclonal antibody carrying the same anti PD-L1 arm present in AI3S79/N, induces IL-2 secretion at levels comparable to Atezolizumab. Of note, AI3S79/N is the most active molecule in this assay as it not only blocks PD-L1, but also provides costimulatory signal to the CD4+ T cells, resulting in better T cell activation (FIG. 11B).

### Example 8: In Vivo Safety Evaluation in a Humanized Mouse Model Sensitive to CD28-Mediated Cytokine Release Syndrome

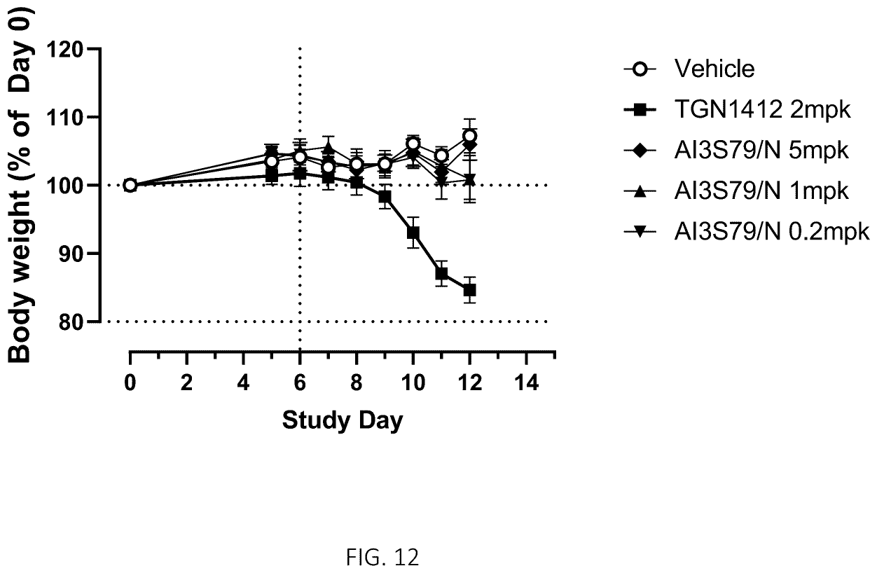

A humanized mouse model sensitive to CD28-mediated CRS was used to assess AD3S79/N safety in vivo. Briefly, nonobese diabetic (NOD) scid gamma (NSG)-Major Histocompatibility Complex (MHC) I/II double knock out (KO) mice were irradiated and engrafted with human PBMCs previously selected for their sensitivity to anti-CD28 superagonist Abs. Six days later, mice were dosed with Abs. As a positive control, a TGN1412 analogue was used. As a negative control the mice were injected with phosphate-buffered saline (PBS) vehicle. Mice were observed daily and subjected to a body weight monitoring and CRS score assessment. Mice reaching the endpoints based on body weight (>20%) or CRS score (>3) were euthanized. As shown in FIG. 12, contrary to mice receiving TGN1412 at 2 mg/kg, which all showed important body-weight loss following drug administration on day 6, treatment with a dose range of AI3S79/N was tolerated by these mice.

### Example 9: In Vitro Single Agent Activity of AI3S79/N in a CMV-Recall Assay

To assess the single-agent activity of AI3S79/N in a physiologically relevant in vitro context, an assay where tumor cells are artificially loaded with a pool of CMV-derived peptides, including the NLV-peptide (a HLA-A*02-restricted epitope derived from the CMV lower matrix protein pp65). These tumor cells are then cocultured with PBMCs from donors which are known to contain NLV-specific T cells. When these T cells reencounter the NLV peptide presented in the context of MHC molecules, they get activated, start proliferating and specifically kill the NLV-loaded target cells, a process that PD-L1xCD28 bsAbs are expected to amplify by providing the signal 2 for T cell activation. Killing was quantified by measuring the remaining living target cells by CellTiter-Glo® luminescent cell viability assay, while the expansion of NLV-specific T cells was quantify using a fluorescently labelled HLA-A*02-NLV tetramer.

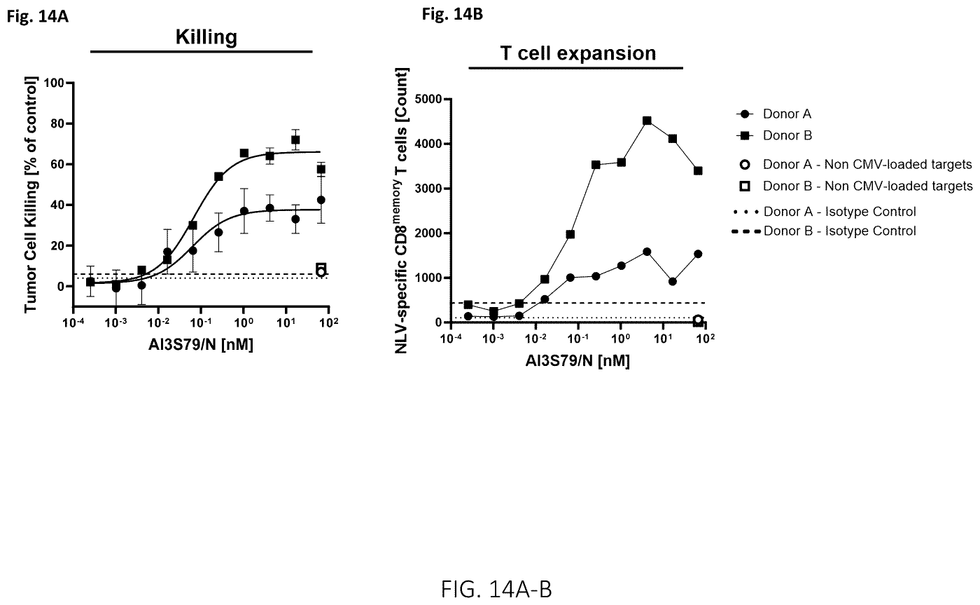

The PD-L1-positive MDA-MB-231 cell line was first loaded with a pool of CMV-derived peptides and then cocultured with two different PBMC donors containing CMV-reactive T cells. For both donors, a AI3S79/N-induced dose-dependent killing of the NLV-loaded target cells was observed (FIG. 14A). Correspondingly, a AI3S79/N-induced dose-dependent expansion of NLV-specific T cells was also observed (FIG. 14B). Importantly, when target cells were not loaded with CMV peptides and thus unable to provide T cell signal 1, they were not killed and T cells did not proliferate in presence of AI3S79′N (Non CMV-loaded controls on both panels).

### Example 10: In Vivo Efficacy Studies in Combination with a T Cell Engager (TCE) in TCE-Resistant Models

In Vivo Efficacy Study of AI3S79/N in Combination with a CEAxCD3 TCE in the Treatment of HPAC Tumors Engrafted in PBMC-Humanized NOG Mice

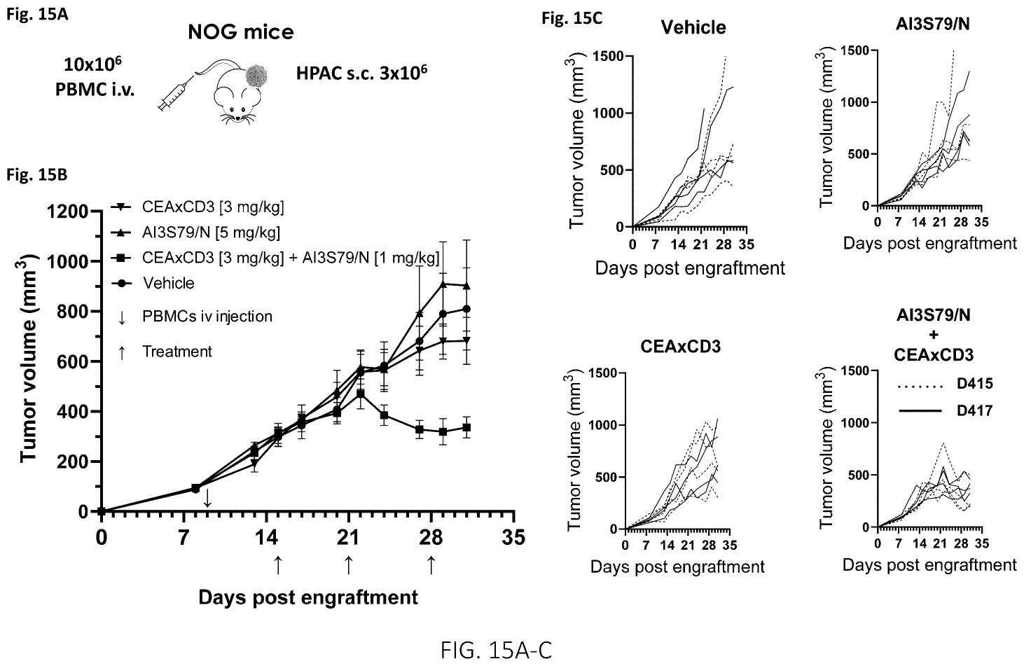

The synergy between AI3S79/N and CEAxCD3 observed in vitro (example 4) was confirmed in vivo in a model where the human pancreatic adenocarcinoma epithelial cell line HPAC was subcutaneously engrafted into PBMC-humanized NOG mice (FIG. 15A). Briefly: 3×10⁶ HPAC cells were subcutaneously engrafted into NOG mice (Taconic). When the average tumor volume reached 100 mm³, mice were humanized i.v. with 10×10⁶ PBMC cells. Two donors were used, and each group eventually contained 4 mice for each donor, for a total of 8 mice per group. Treatment started on day 15 when the average tumor volume was close to 300 mm³ and consisted of 3 injections of vehicle, CEAxCD3 (3 mg/kg), AI3S79/N (5 mg/kg) or a combination of both CEAxCD3 and A3S79/N (dosed at 3 mg/kg or 1 mg/kg, respectively) occurring on day 15, 21 and 28. Tumors were measured 3 times weekly by digital caliper until the endpoint of the experiment (tumor volume 1500 mm³) or appearance of GvHD symptoms. Tumor volume was calculated using the formula (length×width²)×0.5.

The experiment had to be stopped due to the appearance of GvHD symptoms on day 31. Before that and starting on day 24, the average tumor volume of the combination group started to decrease (FIG. 15B) and tumor regression or stasis was observed in all mice treated with the combination (FIG. 15C), suggesting that AI3S79/N synergized with CEAxCD3 to induce a strong anti-tumor response.

In Vivo Efficacy Study of A3S79/N in Combination with a CEAxCD3 TCE in the Treatment of HPAF-II Tumors in Fully Humanized BRGSF-HIS Mice

The synergy between AI3S79/N and CEAxCD3 was further assessed in a second model where the human pancreatic adenocarcinoma cell line HPAF-II was subcutaneously engrafted into fully humanized BRGSF-HIS mice. Briefly, 1.5×10⁶ (CEA+/PD-L1+HPAF-II cells were engrafted into hFlt-3L boosted BRGSF-HIS mice (genOway). Each group contained 2 mice for each of the 6 hematopoietic stem cell donors used, for a total of 12 mice per group. When the average tumor volume reached 65 mm3 (day 8 post engraftment), treatment began. Mice received 3 doses of vehicle, CEAxCD3 (10 mg/kg), or a combination of CEAxCD3+AI3S79/N (10+10 mg/kg) on day 8, 11 and 14. Tumors were measured 2-3 times weekly by digital caliper until the endpoint of the experiment (tumor volume=1500 mm³). Tumor volume was calculated using the formula (length×width²)×0.5.

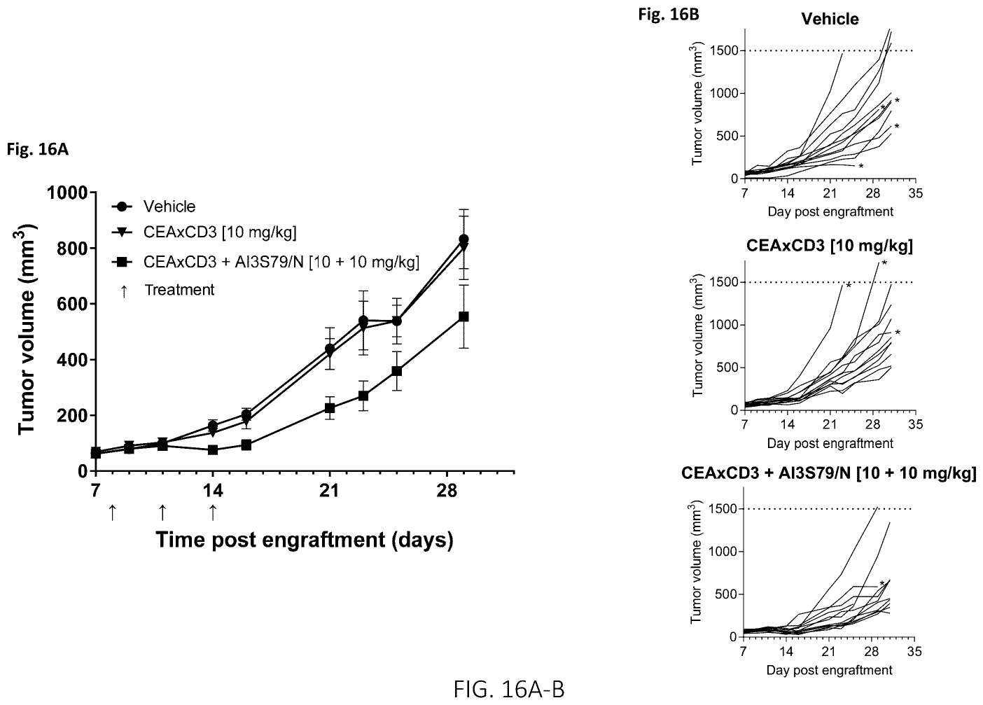

Contrary to CEAxCD3 alone, which showed no anti-tumor activity in this model, the CEAxCD3+AI3S79/N combination resulted in reduced tumor progression (FIG. 16A). The tumor growth kinetic of individual mice is shown in FIG. 16B.

### Example 111: Single and Repeat-Dose PK and Tolerability Study in Cynomolgous Monkey

A study was conducted in cynomolgus monkeys to investigate the PK and tolerability of AI3S79/N after single and repeated intravenous injections. The study encompassed 3 groups: (a) single iv injection of A3S79/N at 0.5 mg/kg; (b) single iv injection of A3S79/N at 10 mg/kg; (c) repeated (n=2) iv injection of AI3S79/N at 10 mg/kg followed by histopathological examination.

### Pharmacokinetics

The serum levels of A3S79/N were quantified in the serum of cynomolgus monkeys using a validated generic pharmacokinetic assay based on Meso-Scale Discovery (MSD) technology. Briefly, a biotinylated anti-human CH2 was coated on a streptavidin MSD plate to capture AI3S79/N from samples. Detection was allowed by SulfoTag-coupled anti-human CH2. Signals were acquired on Meso Sector S600 instrument and concentrations extrapolated against standard curve of AI13S79/N. Upon quantification of AI3S79/N in the samples, pharmacokinetic data evaluation was conducted according to standard non-compartmental analysis using SAS software version 9.4

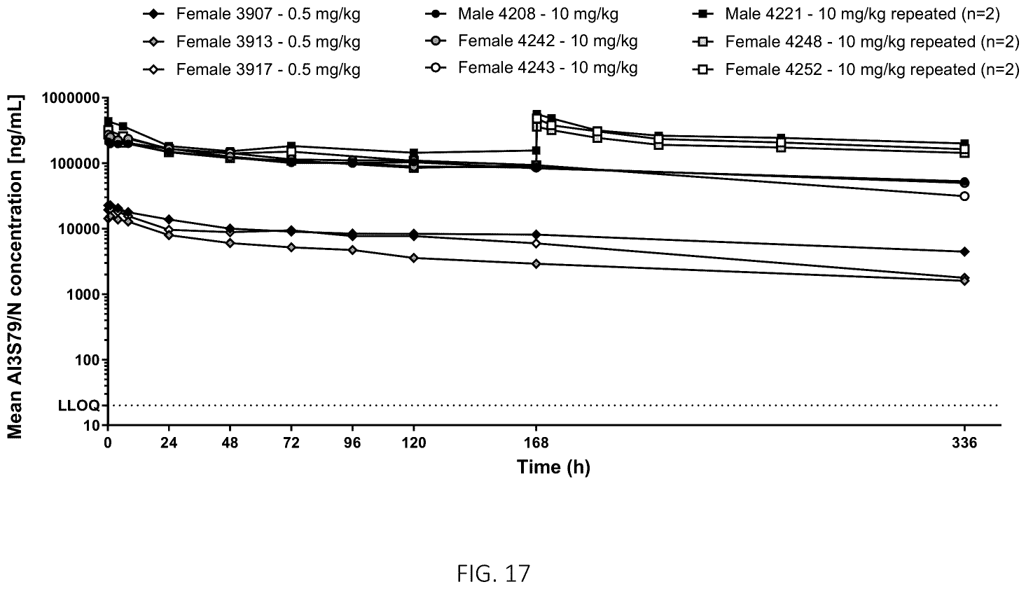

The AI3S79/NT concentration-versus-time curves obtained after bioanalytical testing are shown in FIG. 17. AI3S79/N PK was close to dose-proportional, with mean Cₘₐₓ, AUC_{inf} and AUC₀₋₁₆₈ₜₕ values 13-23 times higher after administration of 10 mg/kg compared to 0.5 mg/kg.

Accumulation of AI3S79/N was observed after the repeated injection at 10 mg/kg, since in monkeys treated twice mean Cₘₐₓ and AUC₀₋₁₆₈ₜₕ values were 1.4 and 1.6 times higher, respectively, on Day 8 compared to Day 1.

### Tolerability

Single and repeated intravenous administration of A3S79/N was well tolerated. No mortality and no abnormal clinical signs were noted throughout the study. No treatment-related changes were reported in body weights, food intake, clinical pathology and at postmortem examinations.

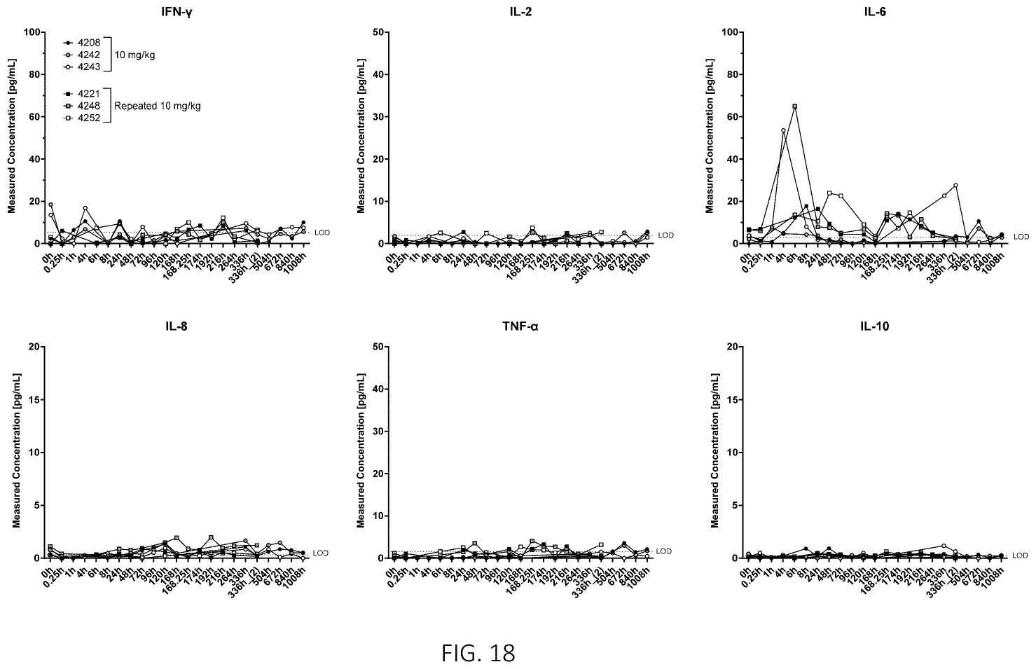

The quantification of serum cytokines (IFNγ, TNFα, IL-2, IL-6, IL-8 and IL-10) with MesoScale Discovery (MSD) was performed in samples collected throughout the study from selected groups (FIG. 18). No increase in the levels of IFNγ, TNFα, L1-2, IL-8 and IL-10 was observed following single or repeated treatment with AI3S79/N at both dose levels. A transient increase in IL-6 was observed after the first dose, at the 4 or 8-hour timepoint. The increase in IL-6 levels after the second administration of A3S79/N was milder than after the first administration for all three monkeys.

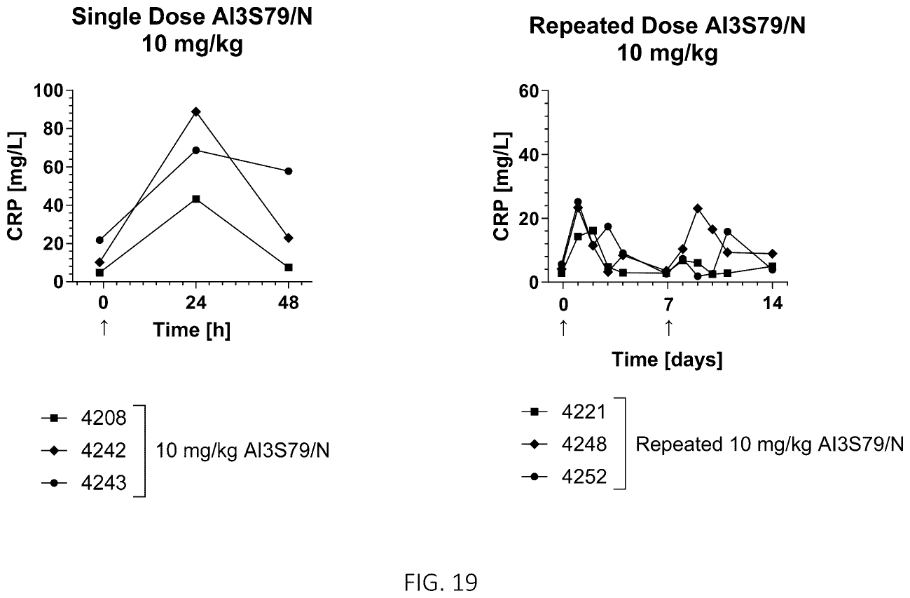

The levels of C-reactive protein (CRP) in sera of the treated monkeys were measured by ELISA (Life Diagnostics Inc.). A transient minimal increase in CRP was observed in most animals after the first injection, starting at 8 h post dose, peaking at 24 h and resolving after approximately 4-5 days. In animals receiving 2 doses, the second injection was followed by a similar transient CRIP increase in only one animal and with delayed and lower maximum level in another one, suggesting, a lower AI3S79/N-related increase of this marker following repeated injections (FIG. 19).

Overall, changes in serum biomarkers were restricted to IL-6 and CRP and were mild in magnitude and transient. Within 24-48 h (IL-6) or 2 to 3 days (CRP) the levels were back to pre-dose for all animals, without anti-inflammatory medication.

In conclusion, single and repeated intravenous administration of AI3S79/N was well tolerated. No abnormalities in clinical signs and food intake were observed, and no treatment related meaningful changes were present in body weights, clinical pathology and anatomic pathology.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Correnti, Colin E., George S. Laszlo, Willem J. de van der Schueren, Colin D. Godwin, Ashok Bandaranayake, Melanie A. Busch, Chelsea J. Gudgeon, et al. 2018. ‘Simultaneous Multiple Interaction T-Cell Engaging (SMITE) Bispecific Antibodies Overcome Bispecific T-Cell Engager (BiTE) Resistance via CD28 Co-Stimulation’, Leukemia 32 (5): 1239-43. https://doi.org/10.1038/s41375-018-0014-3.
Holliger, Philipp, Oliver Manzke, Mary Span, Robert Hawkins, Bernd Fleischmann, Liu Qinghua, Jürgen Wolf, Volker Diehl, Olivier Cochet, and Greg Winter. 1999. ‘Carcinoembryonic Antigen (CEA)-Specific T-Cell Activation in Colon Carcinoma Induced by Anti-CD3×Anti-CEA Bispecific Diabodies and B7×Anti-CEA Bispecific Fusion Proteins’. Cancer Research 59 (12): 2909-16.
Römer, Paula S., Susanne Berr, Elita Avota, Shin-Young Na., Manuela Battaglia, Ineke ten Berge, Hermann Einsele, and Thomas Hünig. 2011. ‘Preculture of PBMCs at High Cell Density Increases Sensitivity of T-Cell Responses, Revealing Cytokine Release by CD28 Superagonist TGN-142’. Blood 118 (26): 6772-82. https://doi.org/10.1182/blood-2010-12-319780.
Skokos, Dimitris, Janelle C. Waite, Lauric Haber, Alison Crawford, Aynur Hermann, Erica Ullman, Rabih Slim, et al. 2020. ‘A Class of Costimulatory CD28-Bispecific Antibodies That Enhance the Antitumor Activity of CD3-Bispecific Antibodies’. Science Translational Medicine 12 (525): eaaw7888. htps://doi.org/10.1126/scitranslmed.aaw7888.
Stebbings, Richard, Lucy Findlay, Cherry Edwards, David Eastwood, Chris Bird, David North, Yogesh Mistry, et al. 200⁷, ‘“Cytokine Storm” in the Phase I Trial of Monoclonal Antibody TGN1412: Better Understanding the Causes to Improve PreClinical Testing of Immunotherapeutics’. The Journal of Immunology 179 (5): 3325-31. https://doi.org/10.4049/jimimunol.179.5.3325,
Waite, Janelle C., Bei Wang, Lauric Haber, Aynur Hermann, Erica Ullman, Xuan Ye, Drew Dudgeon, et al. 2020. ‘Tumor-Targeted CD28 Bispecific Antibodies Enhance the Antitumor Efficacy of PD-1 Immunotherapy’. Science Translational Medicine 12 (549): eaba232.5. https://doi.org/10.1126/scitranslmed.aba2325.

1. A bispecific antibody comprising:
a first antigen binding domain that binds to Programmed Death-ligand 1 (PD-L1); wherein the first antigen binding domain comprises:
i. a first heavy chain variable region having a complementarity determining region 1 (CDR1) comprising the amino acid sequence of SEQ ID NO: 6; a complementarity determining region 2 (CDR2) comprising the amino acid sequence of SEQ ID NO: 7; and a complementarity determining region 3 (CDR3) comprising the amino acid sequence of SEQ ID NO:8; and
ii. a first light chain variable region having:
a CDR1 comprising the amino acid sequence of SEQ ID NO: 13; a CDR2 comprising the amino acid sequence of SEQ ID NO: 14; and a CDR3 comprising the amino acid sequence of SEQ ID NO: 15; and
a second antigen binding domain that binds CD28, wherein the second antigen binding domain comprises:
i. a second heavy chain variable region having a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8.

2. The bispecific antibody of claim 1, wherein the second antigen binding domain comprises:
a second light chain variable region having:
a) a CDR1 comprising an amino acid sequence of SEQ ID NO: 18;
a CDR2 comprising an amino acid sequence of SEQ ID NO: 19; and
a CDR3 comprising an amino acid sequence of SEQ ID NO: 20; or
b) a CDR1 comprising an amino acid sequence of SEQ ID NO: 23;
a CDR2 comprising an amino acid sequence of SEQ ID NO: 24; and
a CDR3 comprising an amino acid sequence of SEQ ID NO: 25.

3. The bispecific antibody of claim 1 or 2, wherein the first and second heavy chain variable region comprises the amino acid sequence of SEQ ID NO: 9.

4. The bispecific antibody of claim 1 or 2, wherein the first and second heavy chain comprises the amino acid sequence of SEQ ID NO: 11 or SEQ ID NO: 12.

5. The bispecific antibody of claim 1, wherein the first light chain variable region of part ii comprises the amino acid sequence of SEQ ID NO: 16.

6. The bispecific antibody of claim 1, wherein the first light chain of part ii comprises the amino acid sequence of SEQ ID NO: 17.

7. The bispecific antibody of claim 2, wherein the second light chain variable region of
1. part (a) comprises the amino acid sequence of SEQ ID NO: 21; and
2. part (b) comprises the amino acid sequence of SEQ ID NO: 26.

8. The bispecific antibody of claim 2, wherein the second light chain of
1. part (a) comprises the amino acid sequence of SEQ ID NO: 22; and
2. part (b) comprises the amino acid sequence of SEQ ID NO: 27.

9. The bispecific antibody of claim 1 or 2, wherein the first light chain is a kappa and the second light chain is a lambda.

10. The bispecific antibody of claim 1 or 2, wherein the first light chain is a lambda and the second light chain is a kappa.

11. The bispecific antibody of claim 1, wherein the bispecific antibody comprises an Fc domain comprising one or more amino acid substitutions that reduce binding to an activating Fc receptor and/or reduce effector function.

12. The bispecific antibody of claim 11, wherein the amino acid substitution comprises a L234A and L235A substitution.

13. The bispecific antibody of claim 11, wherein the amino acid substitution comprises a P329A, P329G or P329R substitution.

14. The bispecific antibody of claim 1 or 2, wherein the antibody has an IgG isotype.

15. The bispecific antibody of claim 1 or 2, wherein the antibodies are human.

16. The bispecific antibody of claim 1 or 2, wherein the composition enables PD-L1-dependent T cell activation.

17. A composition comprising the bispecific antibody of claim 1 or 2.

18. A method of reducing the proliferation of and/or enhancing the killing of a tumor cell comprising contacting the cell with the composition according to claim 17.

19. A method of treating a cancer in a subject comprising administering to the subject the composition according to claim 17.
